(19) United States
(12) Patent Application Publication
(10) Pub. No.: US 2011/0293686 A1
(43) Pub. Date: Dec. 1, 2011

(54) Anti-viral compositions and methods for administration

(75) Inventors: Roderick A. Hyde, Redmond, WA (US); Muriel Y. Ishikawa, Livermore, CA (US); Jordin T. Kare, Seattle, WA (US); Eric C. Leuthardt, St. Louis, MO (US); Lowell L. Wood, JR., Bellevue, WA (US); Victoria Y.H. Wood, Livermore, CA (US)

(73) Assignee: Searete LLC, a limited liability corporation of the State of Delaware

(21) Appl. No.: 12/802,144

(22) Filed: May 28, 2010

Related U.S. Application Data
(63) Continuation-in-part of application No. 12/802,145, filed on May 28, 2010.
Application No. 12/802,145 is a continuation-in-part of application No. 12/802,143, filed on May 28, 2010.
Application No. 12/802,143 is a continuation-in-part of application No. 12/802,146, filed on May 28, 2010.

(51) Int. Cl. A61K 9/00; A61M 37/00; A61P 31/12; A61M 31/00; A61F 6/06

(52) U.S. Cl. 424/423; 424/400; 424/422; 424/430; 604/890.1; 604/65

(57) ABSTRACT

Certain embodiments disclosed relate to compositions, including therapeutic compositions, methods, articles of manufacture, systems, and devices. Certain embodiments relate to anti-viral compositions, methods, articles of manufacture, systems and devices.

### CROSS-REFERENCE TO RELATED APPLICATIONS

The present application is related to and claims the benefit of the earliest available effective filing date(s) from the following listed application(s) (the “Related Applications”) (e.g., claims earliest available priority dates for other than provisional patent applications or claims benefits under 35 USC §119(e) for provisional patent applications, for any and all parent, grandparent, great-grandparent, etc. applications of the Related Application(s)).

### RELATED APPLICATIONS

For purposes of the USPTO extra-statutory requirements, the present application constitutes a continuation-in-part of U.S. patent application Ser. No. to be assigned, Docket No. 0508-004-017-000000, entitled ANTI-VIRAL COMPOSITIONS AND METHODS FOR ADMINISTRATION, naming Roderick A. Hyde, Muriel Y. Ishikawa, Jordin T. Kare, Eric C. Leuthardt, Lowell L. Wood, Jr. and Victoria Y. H. Wood as inventors, filed 28 May 2010, which is currently co-pending, or is an application of which a currently co-pending application is entitled to the benefit of the filing date.

For purposes of the USPTO extra-statutory requirements, the present application constitutes a continuation-in-part of U.S. patent application Ser. No. to be assigned, Docket No. 0508-004-017B-000000, entitled ANTI-VIRAL COMPOSITIONS AND METHODS FOR ADMINISTRATION, naming Roderick A. Hyde, Muriel Y. Ishikawa, Jordin T. Kare, Eric C. Leuthardt, Lowell L. Wood, Jr. and Victoria Y. H. Wood as inventors, filed 28 May 2010, which is currently co-pending, or is an application of which a currently co-pending application is entitled to the benefit of the filing date.

For purposes of the USPTO extra-statutory requirements, the present application constitutes a continuation-in-part of U.S. patent application Ser. No. to be assigned, Docket No. 0508-004-017C-000000, entitled ANTI-VIRAL COMPOSITIONS AND METHODS FOR ADMINISTRATION, naming Roderick A. Hyde, Muriel Y. Ishikawa, Jordin T. Kare, Eric C. Leuthardt, Lowell L. Wood, Jr. and Victoria Y. H. Wood as inventors, filed 28 May 2010, which is currently co-pending, or is an application of which a currently co-pending application is entitled to the benefit of the filing date.

### SUMMARY

The present disclosure relates to therapeutic compositions, methods of administering or using the same, devices for administering or using the same, articles of manufacture of the same, and computer systems, computer-implemented methods, and related products thereof.

In an embodiment, a therapeutic composition comprises an effective amount of at least one virus entry inhibitor in a first formulation; an effective amount of at least one viral-replication modulator in a second formulation; and at least one pharmaceutically-acceptable carrier or excipient; wherein the first formulation regulates the release of the at least one virus entry inhibitor and the second formulation regulates the release of the at least one viral-replication modulator; and wherein the maximum concentration in a biological fluid of the at least one virus entry inhibitor occurs at a time point approximately prior to the maximum concentration of the at least one viral-replication modulator.

In an embodiment, a therapeutic composition, comprises an effective amount of least one virus entry inhibitor in a first formulation; an effective amount of at least one viral-replication modulator in a second formulation; and at least one pharmaceutically-acceptable carrier or excipient; wherein the first formulation regulates the release of the at least one virus entry inhibitor and the second formulation regulates the release of the at least one viral-replication modulator; and wherein the effective amount in a biological fluid of the at least one virus entry inhibitor occurs at a time point approximately prior to the effective amount of the at least one viral-replication modulator. In an embodiment, the first formulation includes both an immediate release component as well as a sustained release component. In an embodiment, the second formulation includes at least one of a delayed release component or a sustained release component. In an embodiment, the release kinetics of the first formulation and the second formulation are configured for an immediate release of a higher ratio of the at least one virus entry inhibitor relative to the at least one viral-replication modulator. In an embodiment, the release kinetics of the first formulation and the second formulation are configured for an immediate release of at least about 2-fold, at least about 3-fold, at least about 4-fold, at least about 5-fold, at least about 6-fold, at least about 7-fold, at least about 8-fold, at least about 9-fold, at least about 10-fold, or any value therebetween or greater, higher ratio of the at least one virus entry inhibitor relative to the at least one viral-replication modulator. In an embodiment, the at least one virus entry inhibitor and the at least one viral-replication modulator are formulated for the same virus. In an embodiment, the at least one virus entry inhibitor and the at least one viral-replication modulator are formulated for the same strain of virus. In an embodiment, the at least one virus entry inhibitor and the at least one viral-replication modulator are formulated for different strains of virus, or different viruses.

In an embodiment, the therapeutic composition further comprises at least one detection indicator. For example, the detection indicator includes, but is not limited to, at least one of a colorimetric reaction, fluorescent reaction, magnetic reaction, luminescent reaction, chemical reaction, biological reaction, electrochemical reaction, or other reactivity. In an embodiment, the at least one detection indicator is formulated to be detected in at least one biological fluid or tissue (e.g., including but not limited to skin, oral cavity, ocular cavity, eyelid, retina, sweat, saliva, blood, urine, vaginal fluid, lacrimal secretions, mucus, or other fluid or tissue).

In an embodiment, a therapeutic composition comprises an effective amount of at least one virus entry inhibitor in a first formulation; an effective amount of at least one viral-replication modulator in a second formulation; and at least one pharmaceutically-acceptable carrier or excipient; wherein the first formulation regulates the release of the at least one virus entry inhibitor and the second formulation regulates the release of the at least one viral-replication modulator; and wherein the effective amount in a biological fluid of the at least one virus entry inhibitor occurs at a time point approximately prior to the effective amount of the at least one viral-replication modulator.

In an embodiment, a method comprises administering to an asymptomatic subject infected or at risk of infection with at least one virus, an effective amount of a therapeutic composition; the therapeutic composition including an effective amount of at least one virus entry inhibitor in a first formulation; an effective amount of at least one viral-replication modulator in a second formulation; and at least one pharmaceutically-acceptable carrier or excipient; wherein the first formulation regulates the release of the at least one virus entry inhibitor and the second formulation regulates the release of the at least one viral-replication modulator; and wherein the maximum concentration in a biological fluid of the at least one virus entry inhibitor occurs at a time point approximately prior to the maximum concentration of the at least one viral-replication modulator.

In an embodiment, a method comprises administering to a biological tissue infected or at risk of infection with at least one virus, an effective amount of a therapeutic composition; the therapeutic composition including an effective amount of at least one virus entry inhibitor in a first formulation; an effective amount of at least one viral-replication modulator in a second formulation; and at least one pharmaceutically-acceptable carrier or excipient; wherein the first formulation regulates the release of the at least one virus entry inhibitor and the second formulation regulates the release of the at least one viral-replication modulator; and wherein the maximum concentration in a biological fluid of the at least one virus entry inhibitor occurs at a time point approximately prior to the maximum concentration of the at least one viral-replication modulator.

In an embodiment, a method comprises administering to an unborn offspring subject infected or at risk of infection with at least one virus, an effective amount of a therapeutic composition; the therapeutic composition including an effective amount of at least one virus entry inhibitor in a first formulation; an effective amount of at least one viral-replication modulator in a second formulation; and at least one pharmaceutically-acceptable carrier or excipient; wherein the first formulation regulates the release of the at least one virus entry inhibitor and the second formulation regulates the release of the at least one viral-replication modulator; and wherein the maximum concentration in a biological fluid of the at least one virus entry inhibitor occurs at a time point approximately prior to the maximum concentration of the at least one viral-replication modulator.

In an embodiment, a drug delivery device comprises a housing including at least one reservoir containing at least one therapeutic composition, the at least one reservoir configured to deliver at least a portion of the at least one therapeutic composition to at least one biological tissue, wherein the at least one therapeutic composition includes at least one virus entry inhibitor in a first formulation; at least one viral-replication modulator in a second formulation; wherein the maximum. concentration in a biological fluid of the at least one virus entry inhibitor occurs at a time point approximately prior to a time point at which the at least one viral-replication modulator reaches a maximum concentration.

In an embodiment, an article of manufacture comprises an article configured to contact at least one biological tissue of a subject; and configured to deliver at least one therapeutic composition including at least one virus entry inhibitor and at least one viral-replication modulator; wherein the maximum concentration in a biological fluid of the at least one virus entry inhibitor occurs at a time point approximately prior to a time point at which the at least one viral-replication modulator reaches a maximum concentration.

In an embodiment, a system comprises at least one computing device; at least one drug delivery device configured to dispense at least a portion of a therapeutic composition to at least one subject infected or at risk for infection with at least one virus; and one or more instructions that when executed on a computing device cause the computing device to regulate the dispensing of the at least one therapeutic composition from the at least one drug delivery device, wherein the at least one therapeutic composition includes at least one virus entry inhibitor in a first formulation; at least one viral-replication modulator in a second formulation; wherein the maximum concentration in a biological fluid of the at least one virus entry inhibitor occurs at a time point approximately prior to a time point at which the at least one viral-replication modulator reaches a maximum concentration.

In an embodiment, a system comprises circuitry for regulating dispensing at least a portion of a therapeutic composition from at least one drug delivery device, the at least one therapeutic composition including at least one virus entry inhibitor in a first formulation, and at least one viral-replication modulator in a second formulation; wherein the maximum concentration in a biological fluid of the at least one virus entry inhibitor occurs at a time point approximately prior to a time point at which the at least one viral-replication modulator reaches a maximum concentration.

In an embodiment, a computer-implemented method comprises one or more instructions for regulating dispensing at least a portion of a therapeutic composition from at least one drug delivery device, the at least one therapeutic composition including at least one virus entry inhibitor in a first formulation, and at least one viral-replication modulator in a second formulation; wherein the maximum concentration in a biological fluid of the at least one virus entry inhibitor occurs at a time point approximately prior to a time point at which the at least one viral-replication modulator reaches a maximum concentration.

In an embodiment, a computer program product comprises one or more signal-bearing media bearing one or more instructions that, when executed on a computing device, cause the computing device to implement a method including: regulating dispensing at least a portion of a therapeutic composition from at least one drug delivery device, the at least one therapeutic composition including at least one virus entry inhibitor in a first formulation, and at least one viral-replication modulator in a second formulation; wherein the maximum concentration in a biological fluid of the at least one virus entry inhibitor occurs at a time point approximately prior to a time point at which the at least one viral-replication modulator reaches a maximum concentration.

The foregoing summary is illustrative only and is not intended to be in any way limiting. In addition to the illustrative aspects, embodiments, and features described above, further aspects, embodiments, and features will become apparent by reference to the drawings and the following detailed description.

Anti-viral compositions, methods of administration, delivery devices related thereto, and computer-implemented methods, programs, and systems are described herein.

### BRIEF DESCRIPTION OF THE FIGURES

FIG. 1 illustrates an example of the fluid concentration over time of a therapeutic composition.

FIG. 2 illustrates the fluid concentration over time of a therapeutic composition administered to a subject intravenously (IV) or orally (Oral).

FIG. 3 illustrates the fluid concentration over time of a therapeutic composition.

FIG. 4 illustrates the fluid concentration over time of a therapeutic composition.

FIG. 5 illustrates the fluid concentration over time of a therapeutic composition.

FIG. 6 illustrates an article of manufacture including various embodiments described herein.

FIG. 7 illustrates a partial view of various embodiments of a delivery device disclosed herein.

FIG. 8 illustrates a partial view of various embodiments of FIG. 7.

FIG. 9 illustrates a partial view of various embodiments of FIG. 7.

FIG. 10 illustrates a partial view of various embodiments of FIG. 7.

FIG. 11 illustrates a partial view of various embodiments of a system disclosed herein.

FIG. 12 illustrates a partial view of various embodiments of FIG. 11.

FIG. 13 illustrates a partial view of various embodiments of FIG. 11.

FIG. 14 illustrates a partial view of various embodiments of FIG. 11.

FIG. 15 illustrates a partial view of various embodiments of a system disclosed herein.

FIG. 16 illustrates a partial view of various embodiments of a computer-implemented method disclosed herein.

FIG. 17 illustrates a partial view of various embodiments of FIG. 16.

FIG. 18 illustrates a partial view of various embodiments of FIG. 16.

FIG. 19 illustrates a partial view of a computer program product disclosed herein.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

The present application uses formal outline headings for clarity of presentation. However, it is to be understood that the outline headings are for presentation purposes, and that different types of subject matter may be discussed throughout the application (e.g., method(s) may be described under composition heading(s) and/or kit headings; and/or descriptions of single topics may span two or more topic headings). Hence, the use of the formal outline headings is not intended to be in any way limiting.

In an embodiment, anti-viral therapeutic compositions, and methods and devices for delivering the same are disclosed. In an embodiment, a therapeutic composition is administered to a subject infected, or at risk of being infected, with at least one virus.

In an embodiment, a therapeutic composition includes an effective amount of at least one virus entry inhibitor in a first formulation; an effective amount of at least one viral-replication modulator in a second formulation; and at least one pharmaceutically-acceptable carrier or excipient; wherein the first formulation regulates the release of the at least one virus entry inhibitor and the second formulation regulates the release of the at least one viral-replication modulator; and wherein the maximum concentration in a biological fluid of the at least one virus entry inhibitor occurs at a time point approximately prior to the maximum concentration of the at least one viral-replication modulator.

Thus, in an embodiment, the virus entry inhibitor reaches its maximum biological fluid concentration (e.g., maximum blood serum concentration) sooner than the other anti-viral component(s) present in the composition. In this way, the rapid or immediate dose virus entry inhibitor provides maximal prophylactic protection from the initial infection of a cell by a virus, while the other anti-viral component(s) are released secondarily or at a slower rate, in order to act on any cells that are inadvertently infected by virus particles.

In an embodiment, the rate of dissolution of the virus entry inhibitor formulation is greater than the rate of dissolution of the viral-replication modulator formulation of the therapeutic composition. In an embodiment, the rate of absorption of the virus entry inhibitor formulation is greater than the rate of absorption of the viral-replication modulator formulation of the therapeutic composition.

Without wishing to be bound to any particular theory of mechanism relating to virus infection or transmission, in some instances particular viruses infect specific cell types. For example, enveloped viruses generally initiate infection by attachment of the virus to a host cell receptor, followed by membrane fusion between the viral and cellular plasma or endosomal membranes. Typically, the fusion proteins of different viruses are synthesized as precursors with a transmembrane domain near the carboxyl terminus, and are cleaved into two disulfide-linked subunits by cellular proteases. See, for example, Okazaki, and Kida, J. Gen. Virol., vol. 85, pp. 2131-2137 (2004), which is incorporated herein by reference. Generally, a hydrophobic stretch of amino acids at the N-terminus of the subunit containing the transmembrane domain serves as the fusion domain. Id. Two intervening emphipathic heptad repeat regions have been identified adjacent to the transmembrane and fusion domains in the fusion proteins, for example, of orthomyxoviruses, paramyxoviruses, and retroviruses. Id. As reported, synthetic peptides derived from the heptad repeat region of paramyxoviruses and HIV inhibit virus fusion and infection. Id.

In an embodiment, peptide inhibitors of viral infectivity or transmissibility are designed and synthesized according to known or ascertainable virus structural elements. For example, Class I viral fusion proteins, such as those encoded by influenza virus and HIV, contain two prominent alpha helices. Peptides that mimic portions of these alpha helices inhibit structural rearrangements of the fusion proteins and prevent viral infection. See, for example, Hrobowski, et al., Virol. J. vol. 2, pp. 49-59 (2005), which is incorporated herein by reference. The envelope glycoprotein (E) of Flaviviruses, such as West Nile virus and Dengue Virus, are class II viral fusion proteins comprised predominantly of beta sheets. Id. Using a physio-chemical algorithm, the Wimley-White interfacial hydrophobicity scale (WWIHS) is used in combination with known structural data to identify potential peptide inhibitors of West Nile virus and Dengue virus infectivity that target the viral E protein. Id. Likewise, in an embodiment, peptide inhibitors are designed or synthesized by utilizing known viral structural information. For example, peptide inhibitors generated in silico can be tested for physical, biological, or chemical elements, hydrophobicity, or thermodynamic principles, prior to or instead of testing in vitro. Id.

Without wishing to be bound by any particular mechanistic theory, HIV infection likely involves a process mediated by the gp41 and gp120 HIV env proteins and the CD4 cell receptor. For example, the generally accepted model of infection includes the viral envelope glycoprotein complex (gp120/gp41) interacting with cell surface receptors on the membrane of the host cell. Following the binding of gp120 to cell receptors (e.g., CD4, possibly in combination with chemokine co-receptors such as CCR4 or CXCR4), a conformational change occurs in the gp120/gp41 complex that results in the insertion of the gp41 protein into the host cell membrane, and mediates membrane fusion. See for example, U.S. Patent Application Publication No. 20060281673, and U.S. Pat. No. 7,456,251, each of which is incorporated herein by reference. In an embodiment, the therapeutic composition includes one or more inhibitors or antagonists to at least one of gp120, gp41, or CD4 receptor.

Evidence also suggests that HIV is capable of utilizing at least one co-receptor for infectivity. In particular, HIV is able to utilize at least one of: CCR1 receptor, CCR4 receptor, CCR5 receptor, CXCR3 receptor, CCR3 receptor, CCR2 receptor, CX3CR1 receptor, or CXCR4 receptor. In an embodiment, the therapeutic composition disclosed herein includes at least one antagonist to one or more chemokine co-receptors utilized for infectivity by HIV.

In an embodiment, the at least one virus entry inhibitor includes at least one biological cell component antagonist. In an embodiment, the at least one biological cell component antagonist includes at least one biological cell receptor antagonist, including but not limited to at least one cytokine or chemokine receptor. In an embodiment, the at least one biological cell receptor antagonist includes at least one of CCR1 receptor antagonist, CCR4 receptor antagonist, CCR5 receptor antagonist, CXCR3 receptor antagonist, CCR3 receptor antagonist, CCR2 receptor, CX3CR1 receptor antagonist, CXCR4 receptor antagonist, or CD4 receptor antagonist. In an embodiment, the biological cell receptor antagonist includes at least one of a cytokine or chemokine receptor antagonist. In an embodiment, the at least one chemokine receptor antagonist includes at least one antagonist of one or more of CCL1, CCL2, CCL3, CCL4, CCL5, CCL6, CCL7, CCL8, CCL9/CCL10, CCL11, CCL12, CCL13, CCL14, CCL15, CCL16, CCL17, CCL18, CCL19, CCL20, CCL21, CCL22, CCL23, CCL24, CCL25, CCL26, CCL27, CCL28, CCL29, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCL17, CXCL18, CXCL19, CXCL20, CXCL21, CXCL22, XCL1, XCL2, XCL3, XCL4, XCL5, CX3CL1, CX3CL2, or CX3CL3.

In an embodiment, the at least one biological cell receptor antagonist includes at least one of a CD4 receptor antagonist, α4β7 integrin antagonist, α4β1 integrin antagonist, CD209 receptor antagonist, αMβ2 integrin antagonist, or αvβ6 integrin antagonist. In an embodiment, the at least one virus entry inhibitor is derived from at least one of the gp41 or gp120 components of the Human Immunodeficiency Virus.

In an embodiment, an entry inhibitor includes fusion inhibitors, inhibitors of the CD4 receptor, inhibitors of the CCR5 co-receptor, inhibitors of the CXCR4 co-receptor, or inhibitors of other chemokine receptors, or a pharmaceutically acceptable salt or prodrug thereof. Some non-limiting examples of entry inhibitors include AMD-070 (AMD-11070; AnorMed), BlockAide/CR (ADVENTRX Pharm.), BMS 806 (BMS-378806; BMS), Enfurvirtide (T-20, R698, Fuzeon), KRH-1636 (Kureha Pharmaceuticals), ONO-4128 (GW-873140, AK-602, E-913; ONO Pharmaceuticals), Pro-140 (Progenics Pharm), PRO-542 and PRO-140 (Progenies Pharm.), SCH-D (SCH-417690; Schering-Plough), T-1249 (R724; Roche/Trimeris), TAK-220, TAK-652 (Takeda Chem. Ind.), TNX-355 (Tanox) and UK-427,857 (Pfizer). See, for example, U.S. Pat. No. 7,244,716, which is incorporated herein by reference.

In an embodiment, the therapeutic composition includes at least one CCR5 receptor antagonist, for example, including a piperidine skeleton as described in U.S. Patent Application Publication No. 20040053936, which is incorporated herein by reference. For example, in an embodiment, the CCR5 receptor antagonist is represented by the formula (I):

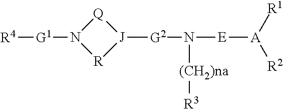

wherein R¹ is a hydrogen atom, a hydrocarbon group optionally having substitutent(s) or a nonaromatic heterocyclic group optionally having substitutent(s), R² is a hydrocarbon group optionally having substituent(s), a nonaromatic heterocyclic group optionally having substituent(s), alternatively R¹ and R² may combine to form, together with A, a heterocyclic group optionally having substituent(s), A is N or N⁺R⁵Y⁻ (wherein R⁵ is a hydrocarbon group and Iris a counter anion), R³ is a cyclic hydrocarbon group optionally having substituent(s) or a heterocyclic group optionally having substituent(s), na is 0 or 1, R⁴ is a hydrogen atom, a hydrocarbon group optionally having substituent(s), a heterocyclic group optionally having substituent(s), an alkoxy group optionally having substituent(s), an aryloxy group optionally having substitutent(s) or an amino group optionally having substituent(s), E is a divalent chain hydrocarbaon group optionally having substituent(s) other than oxo group, G¹ is a bond, CO or SO₂, G² is CO, SO₂NHCO, CONH, or OCO, J is a methane or nitrogen atom, and Q and R are each a bond or a divalent C₁₋₃ chain hydrocarbon optionally having substituent(s), provided that when G² is OCO, J is a methane, and neither Q nor R is a bond, and when G¹ is a bond, neither Q nor R is substituted by oxo group, or a salt thereof. See, for example, U.S. Patent Application Publication No. 20040053936, which is incorporated herein by reference.

In an embodiment, the therapeutic composition includes at least one virus entry inhibitor, and at least one viral-replication modulator. In an embodiment, the at least one virus entry inhibitor modulates at least one of viral fusion with the at least one host cell membrane, or viral internalization by at least one cell. In an embodiment, the at least one viral entry inhibitor is formulated to interfere with or inhibit at least one of viral fusion with at least one cell or viral internalization by at least one cell.

In an embodiment, the at least one virus entry inhibitor includes at least one of maraviroc ((5)-tert-butyl 3-oxo-1-phenylpropylcarbamate), enfuvirtide (Acetyl-YTSLIHSLIEESQNQ QEKNEQELLELDKWASLWNWF-amide), T-22 ([Tyr5, 12, Lys7]-polyphemusin II), ritonavir (1,3-thiazol-5-ylmethyl N-[(2S,3S,5S)-3-hydroxy-5-[(2S)-3-methyl-2-{[methyl({[2-(propan-2-yl)-1,3-thiazol-4-yl]methyl})carbamoyl]amino}butanamido]-1,6-diphenylhexan-2-yl]carbamate), BlockAide/CR™ (NH₂-RIQRGPGRAFVTIGK-COOH), BMS 806 (4-benzoyl-1-[(4-methoxy-1-pyrrolo[2,3-b]pyridine-3-yl)oxoacetyl]-2-R-methylpiperazine), KRH-1636 (N-[(2S)-5-(diaminomethylideneamino)-1-[[(1S)-1-naphthalen-1-ylethyl]amino]-1-oxopentan-2-yl]-4-[(pyridin-2-ylmethylamino)methyl]benzamide), ONO-4128 (4-((4-((3R)-1-butyl-3-((1R)) cyclohexylhy-droxymethyl)-2,5-dioxo-1,4,9-triazaspiro(5.5)undec-9-yl methyl)phenoxy)benzoic acid hydrochloride), Pro-140 (monoclonal antibody for CCR5), Pro-542 (CD4-IgG2 antibody), T-1249 (Ac-WQEWEQKITALLEQAQIQQEKNEYELQKLDKWASLWEWF-NH₂), TAK-220 (1-acetyl-N-{3-[4-(4-carbamoylbenzyl)piperidin-1-yl]propyl}-N-(3-chloro-4-methylphenyl)piperidine-4-carboxamide), TAK-652 ((S)-8-[4-(2-butoxyethoxy)phenyl]-1-isobutyl-N-(4-{[(1-propyl-1H-imidazol-5-yl)methyl]sulfinyl}phenyl)-1,2,3,4-tetrahydro-1-benzazocine-5-carboxamide monomethanesulfonate)), ibalizumab (monoclonal antibody for CD4), TAK-779 (N,N-Dimethyl-N-[4-[2-(4-methylphenyl)-6,7-dihydro-5H-benzocyclohepten-8-ylcarboxamido]benzyl]tetrahydro-2H-pyran-4-aminium chloride), palivizumab (IgG monoclonal antibody), vicriviroc (1-[(4,6-dimethyl-5-pyrimidinyl)carbonyl]-4-[4-[2-methoxy-1(R)-4-(trifluoromethyl)phenyl]ethyl-3(S)-methyl-1-piperazinyl]-4-methylpiperidine), or aplaviroc (4-(4-{[(3R)-1-butyl-3-[(R)-cyclohexylhydroxymethyl]-2,5-dioxo-1,4,9-triazaspiro[5.5]undecan-9-yl]methyl}phenoxy)benzoic acid).

In an embodiment, the at least one virus entry inhibitor is capable of binding to viral hemagglutinin protein. In an embodiment, the at least one virus entry inhibitor includes at least a portion of the signal sequence of a fibroblast growth factor. In an embodiment, the at least one virus entry inhibitor includes at least a portion of the signal sequence of fibroblast growth factor 4.

In an embodiment, the at least one virus entry inhibitor is formulated to alter the pH of at least one endosomal or lysosomal pathway in the at least one cell. In an embodiment, the at least one virus entry inhibitor is formulated to decrease the pH of at least one endosomal or lysosomal pathway in the at least one cell. In an embodiment, the at least one virus entry inhibitor is formulated to increase the pH of at least one endosomal or lysosomal pathway in the at least one cell.

In an embodiment, the at least one virus entry inhibitor includes at least one of a DNA virus entry inhibitor, or RNA virus entry inhibitor. In an embodiment, the at least one virus entry inhibitor includes at least one of a double-stranded DNA virus entry inhibitor, single-stranded DNA virus entry inhibitor, double-stranded RNA virus entry inhibitor, (+) single-strand RNA virus entry inhibitor, (−) single-strand RNA virus entry inhibitor, single-strand RNA-Reverse Transcriptase virus entry inhibitor, or double-stranded DNA-Reverse Transcriptase virus entry inhibitor.

In an embodiment, the at least one virus entry inhibitor includes at least one of human immunodeficiency virus (HIV) type I virus entry inhibitor, HIV-type 2 virus entry inhibitor, simian immunodeficiency virus (SIV) entry inhibitor, or feline leukemia virus entry inhibitor.

The embodiments described herein relate to any infective virus. For example, the at least one virus includes, but is not limited to at least one of human immunodeficiency virus (HIV) type I, HIV-type 2, simian immunodeficiency virus (SW), or feline leukemia virus. In an embodiment, the at least one virus includes, but is not limited to, at least one of picornavirus family, respiratory syncytial virus (RSV), influenza (flu), adenovirus, rhinovirus, enterovirus, poliovirus, rubella virus, paramyxovirus, herpesvirus, rotavirus, neurotropic virus, or oncovirus. In an embodiment, the herpesvirus includes but is not limited to Herpes simplex virus-1, Herpes simplex virus-2, varicella-zoster (chicken pox, shingles, human Herpes virus 3), Epstein-Barr (human Herpes virus 4), cytomegalovirus (human Herpes virus 5), roseolovirus (human Herpes virus 6 and 7), or Karposi's sarcoma-associated herpesvirus (human Herpes virus 8). In an embodiment, the picornavirus family includes but is not limited to picornavirus, poliovirus, rhinovirus, enterovirus (coxsackie virus), hepatitis (hepatitis virus type A, hepatitis virus type B, hepatitis virus type C), aphthovirus, parechovirus, or encephalomyocarditis virus.

In an embodiment, the at least one virus entry inhibitor includes at least one of respiratory syncytial virus (RSV) entry inhibitor, influenza (flu) virus entry inhibitor, adenovirus entry inhibitor, rhinovirus entry inhibitor, enterovirus entry inhibitor, poliovirus entry inhibitor, rubella virus entry inhibitor, paramyxovirus entry inhibitor, herpes simplex virus type I (HSV-1) entry inhibitor, Herpes simplex virus 2 (HSV-2) entry inhibitor, rotavirus entry inhibitor, neurotropic virus entry inhibitor, coxsackie virus entry inhibitor, hepatitis virus type A entry inhibitor, hepatitis virus type B entry inhibitor, hepatitis virus type C entry inhibitor, or oncovirus entry inhibitor.

In an embodiment, the at least one virus entry inhibitor includes one or more of an organic or inorganic small molecule, nucleic acid, amino acid, peptide, polypeptide, protein, glycopeptide, glycoprotein, glycolipid, lipopolysaccharide, peptidoglycan, proteoglycan, lipid, metalloprotein, liposome, or carbohydrate.

In an embodiment, the at least one virus entry inhibitor includes at least one of maraviroc, enfuvirtide, T-22, T-2, AMD-070, BlockAide/CR, BMS 806, KRH-1636, ONO-4128, Pro-140, Pro-542, SCH-D, T-1249, TAK-220, TAK-652, TNX-355, TAK-779, palivizumab, vicriviroc, aplaviroc, AK605, or TAK-779.

In an embodiment, the virus entry inhibitor or viral-replication modulator includes at least one antibody. In an embodiment, the at least one antibody includes at least one of an anti-idiotypic antibody, heteroantibody, antibody fragment, antibody derivative, one or more antibodies linked together, chimeric antibody, humanized antibody, human antibody, recombinant antibody, synthetic antibody, or any part thereof. In an embodiment, the at least one antibody fragment includes at least one of Fc, Fab, Fb, Fv, V domain, H chain, C domain, L chain, or any part thereof.

In an embodiment, the at least one antibody includes at least one anti-hemagglutinin antibody. In an embodiment, the at least one antibody includes human IgG1 CR6261. See, for example, Ekiert, et al., Science vol. 324, pp. 246-251 (2009), which is incorporated herein by reference. Without wishing to be bound by any particular mechanism of action, the antibody CR6261 recognizes a highly conserved helical region in the membrane-proximal stem of hemagglutinin 1 and hemagglutinin 2, and inhibits conformational rearrangements associated with membrane fusion of influenza virus. Id.

An antibody may include an anti-idiotypic antibody, a heteroantibody, multiple antibodies, one or more antibody fragments, one or more antibody derivatives, one or more antibodies linked together, chimeric antibodies, humanized antibodies, human antibodies, recombinant antibodies, synthetic antibodies, or others.

Antibodies or fragments thereof may be generated against a cellular component, such as a receptor or ligand, using standard methods, for example, such as those described by Harlow & Lane (Antibodies: A Laboratory Manual, Cold Spring Harbor Laboratory Press; 1^{st} edition 1988), which is herein incorporated by reference). Alternatively, an antibody fragment directed against an agent may be generated using phage display technology (See, e.g., Kupper et al., BMC Biotechnology, Vol. 5, No. 4, (2005), which is herein incorporated by reference). An antibody or fragment thereof could also be prepared using in silico design (See e.g., Knappik et al., J. Mol. Biol., Vol. 296, pp. 57-86 (2000), which is herein incorporated by reference).

For example, antibodies may be used to either bind to the viral envelope protein or to its respective receptor on the host cell surface, thus blocking the initial interaction of the virus with the host cell. Some non-limiting examples include neutralizing antibodies that bind to a virus and interfere with its ability to infect a cell. Alternatively, the antibody can be reactive against the host cell receptor. One example includes, but is not limited to palivizumab, a monoclonal antibody used in the prevention of respiratory syncytial virus infections in premature infants and/or other infants with medical problems (chronic lung disease, congenital heart disease, immuncompromised, cystic fibrosis). For example, palivizumab targets the fusion protein of RSV, inhibiting its entry into the cell and thereby preventing infection.

Other examples of antibodies that have been in the clinic include, but are not limited to PRO 140, or motavizumab. In an embodiment, an antibody is determined by demonstrating its role by either a viral protein or a host protein in the infection process. For example, antibodies directed against the mannose receptor block the infection of cell by the Dengue virus. See, for example, Miller, et al., PLoS Pathogens vol. 4, pp. 0001-0011 (2008), which is incorporated herein by reference. Similarly, antibodies against α2-β1 and β2 integrins attenuate rotavirus infection of cultured cells. See, for example, Graham, et al., J. Virol. 77:9969-9978, (2003), which is incorporated herein by reference.

In addition or instead of an antibody, the assay may employ another type of recognition element, such as a receptor or ligand binding molecule. Such a recognition element may be a synthetic element like an artificial antibody or peptide mimetic. See e.g., U.S. Pat. No. 5,804,563; U.S. Pat. No. 6,797,522; U.S. Pat. No. 6,670,427; U.S. Pat. No. 5,831,012; U.S. Patent Application 20040018508; Ye and Haupt, Anal Bioanal Chem. vol. 378, pp. 1887-1897, (2004); Peppas and Huang, Pharm Res. vol. 19, pp. 578-587 (2002), each of which is herein incorporated by reference.

Without wishing to be bound by any particular mechanistic pathway, other inhibitors of influenza that may be included in a therapeutic composition described herein include but are not limited to hemagglutinin binding agents (e.g., tert-butyl hydroquinone (TBHQ)); neuraminidase inhibitors; oseltamivir, or zanamivir, which prevent release of nascent virons; or amantadine, which interferes with the M2 channel proton conducting activity. Id.

In an embodiment, the at least one viral-replication modulator is formulated to modulate at least one other virus replication activity (e.g., a viral activity other than viral entry or fusion with the host cell membrane) including one or more of viral transcription, viral replication, viral integration, viral budding, viral release from the at least one cell, or at least one enzymatic activity associated with at least one of these. In an embodiment, modulation of one or more of the listed activities includes interfering with or inhibiting the same. In an embodiment, the at least one enzymatic activity includes, but is not limited to, activity of at least one of a protease, reverse transcriptase, integrase, DNAse, or RNAse. In an embodiment, the at least one viral-replication modulator includes at least one of a defensin, substituted benzimidazole TMC353121, antioxidant, or zinc.

In an embodiment, the therapeutic composition includes at least one viral-replication modulator, including at least one defensin, or agent that modulates defensin production or activity. Defensins are cysteine-rich cationic proteins found in vertebrates and invertebrates that function in defense of bacteria, fungi, or viruses. There are three known forms of mammalian defensins: α-defensins (expressed primarily by neutrophils, natural killer cells, and T cells), β-defensins (primarily secreted by leukocytes and epithelial cells), and θ-defensins (thus far isolated only from leukocytes of certain primates). In particular, α-defensins have known anti-viral properties. For example, α-defensins (α-defensins 1, 2, 3, 4, or any combination thereof) are reported to inhibit HIV replication. See, for example, U.S. Patent Application Publication No. 20040091498, which is incorporated herein by reference.

In an embodiment, the at least one viral-replication modulator includes, but is not limited to, at least one of a protease inhibitor, nucleoside reverse transcriptase inhibitor, nucleotide reverse transcriptase inhibitor, non-nucleoside reverse transcriptase inhibitor, other reverse transcriptase inhibitor, receptor antagonist, or integrase inhibitor. In an embodiment, the integrase inhibitor includes at least one of a diketo acid derivative, bicyclic pyrazole, RSD1624, RSD1625, RSD1996, RSD1997, RSD2196, RSD2197, L-870812, L-731,988, raltegravir, elvitegravir, N-substituted hydroxyl pyrimidinone carboxamide, 4,5-dihydroxypyrimidine-6-carboxamide, or 6-(3-chloro-2-fluorobenzyl)-1-[(2S)-1-hydroxy-3-methylbutan-2-yl]-7-me-thoxy-4-oxo-1,4-dihydroquinoline-3-carboxylic acid. See, for example, U.S. Patent Application Publication No. 20030181490; and U.S. Pat. No. 7,459,452, each of which is incorporated herein by reference.

In an embodiment, the reverse transcriptase inhibitor includes at least one of adefovir dipivoxil, abacavir, zidovudine, nevirapine, delavirdine, etravirne, lamivudine, didanosine (ddL), dideoxyinosine (ddI), enteric coated didanosine, FTC, emtricitabine, 2′,3′-dideoxy-3′-thia-cytidine (3TC), nelfinavir mesylate, NFV, stavudine, 2′,3′-dideoxythymidinene (d4t), loviride, tenofovir disoproxil fumarate, zalcitabine, 2′-3′-dideoxycytidine (ddC), dideoxycytidine, efavirenz, quinolone, pyrrolidone, zidovudine, azidothymidine (AZT), 2′,3′-dideoxy-3′-fluoroadenosine, 2′,3′-dideoxy-3′-fluoroguanasine, 3′ deoxy-3′-fluoro-5-O-[2-(L-valyloxy)-propionyl]guanosine, or ZDV.

In an embodiment, the integrase inhibitor includes 6-(3-chloro-2-fluorobenzyl)-1-[(2S)-1-hydroxy-3-methylbutan-2-YL]-7-methoxy-4-oxo-1,4-dihydroquinoline-3-carboxylic acid, or salt thereof.

In an embodiment, the non-nucloside reverse transcriptase inhibitor includes the compound designated by Formula II:

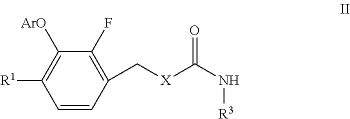

See, for example, U.S. Patent Application No. 20090012034, which is incorporated herein by reference. In an embodiment, X is O or NR²; R¹ is halogen, C₁₋₆ alkyl, C₃₋₇ cycloalkyl, C₁₋₆ haloalkyl, or C₁₋₆ alkoxy; R² and R³ independently are (i) hydrogen or C₁₋₆ alkyl; (ii) R² and R³ together are (CH₂)ₙ, ortho-phenylene, pyridinylene, 3,4-pyridazylene or CH═N wherein n is an integer from 2 to 4 and a nitrogen atom in the pyridinylene or 3,4-pyridazylene ring can be optionally be substituted with an oxygen; or, (iii) R² is hydrogen and R³ is phenyl optionally substituted with one to 3 substituents optionally selected from the group consisting of C₁₋₆ alkyl, C₁₋₆ haloalkyl, C₁₋₆ alkoxy, C₁₋₆ haloalkoxy, halogen, cyano and nitro; Ar is phenyl optionally substituted with 1-3 groups independently selected from the group consisting of selected from halogen, cyano, C₁₋₆ haloalkyl and C₁₋₆ alkyl; or, pharmaceutically acceptable salt(s) thereof. Id.

In an embodiment, the non-nucloside reverse transcriptase inhibitor includes the compound designated by Formula III:

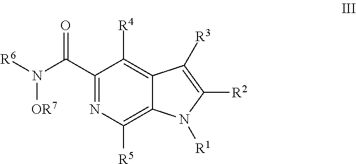

See, for example, U.S. Pat. No. 7,468,375, which is incorporated herein by reference. For example, in an embodiment, R¹ is hydrogen, C₁-C₈ alkyl, C₂-C₈ alkenyl, or C₁-C₈ heteroalkyl, wherein said C₁-C₈ alkyl, C₂-C₈ alkenyl, or C₁-C₈ heteroalkyl groups may be optionally substituted with at least one substituent independently selected from: halo, —OR^{12a}, —N(R^{12a}R^{12b}), —C(O)N(R^{12a})₂, —NR^{12a}C(O)N(R^{12a}R^{12b}), —NR^{12a}C(O)R^{12a}, —NR^{12a}C(NR^{12a})N(R^{12a}R^{12b}), —SR^{12a}, —S(O)R^{12a}, —S(O)₂R^{12a}, —S(O)₂N(R^{12a}R^{12b})², C₁-C₈ alkyl, C₆-C₁₄ aryl, C₃-C₈ cycloalkyl, and C₂-C₉ heteroaryl, wherein said C₁-C₈ alkyl, C₆-C₁₄ aryl, C₃-C₈ cycloalkyl, and C₂-C₉ heteroaryl groups are optionally substituted with at least one substituent independently selected from halo, —C(R^{12a}R^{12b}R^{12c}), —OH, and C₁-C₈ alkoxy; R² is hydrogen; R³ is —(CR⁸R⁹)ₜNR¹⁰R¹¹ or C₁-C₈ heteroalkyl, wherein said C₁-C₈ heteroalkyl is substituted with R²⁴; R⁴ is hydrogen, halo, C₁-C₈ alkyl, —OR^{12a}, —NR^{12a}R^{12b}, C₁-C₈ heteroalkyl, C₂-C₈ alkenyl, or C₂-C₈ alkynyl, wherein said C₂-C₈ alkenyl or C₂-C₈ alkynyl are optionally substituted with at least one R²⁶; R⁵ is hydrogen; R⁶ is hydrogen, C₁-C₈ alkyl, C₁-C₈ heteroalkyl, or C₂-C₈ alkenyl, wherein said C₂-C₈ alkenyl is optionally substituted with at least one —OR^{12a} group; R⁷ is hydrogen, C₁-C₈ heteroalkyl, C₆-C₁₄ aryl, C₂-C₈ alkenyl, or C₁-C₈ alkyl, wherein said C₁-C₈ alkyl is optionally substituted with at least one C₃-C₈ cycloalkyl or C₆-C₁₄ aryl group; each R⁸ and R⁹, which may be the same or different, are independently selected from hydrogen and C₁-C₈ alkyl; R¹⁰ and R¹¹, together with the nitrogen atom to which they are attached, form a C₂-C₉ cycloheteroalkyl group optionally substituted with at least one C₁-C₉ alkyl; each R^{12a}, R^{12b}, R^{12c}, which may be the same or different, is independently selected from hydrogen and C₁-C₈ alkyl; R²⁴ is C₃-C₈ cycloalkyl, C₁-C₈ heteroalkyl, C₂-C₉ cycloheteroalkyl, or C₂-C₉ heteroaryl, each of which is optionally substituted with at least one substituent independently selected from C₁-C₈ alkyl, C₆-C₁₄ aryl, C₂-C₉ heteroaryl, —CF₃, and —OR^{12a}; each R²⁶ is independently selected from —OR^{12a}, halo, C₆-C₁₄ aryl, C₂-C₉ heteroaryl, C₁-C₈ heteroalkyl, C₃-C₈ cycloalkyl, C₂-C₉ cycloheteroalkyl, and —C(R^{12a}R^{12b}R^{12c}); t is an integer from 1 to 3; and pharmaceutically acceptable salts and solvates thereof. Id.

Nucleoside reverse transciptase inhibitors are generally allosteric inhibitors capable of binding reversibly at a nonsubstrate-binding site on a virus reverse transcriptase (e.g., HIV), thereby altering the shape of the active site or blocking polymerase activity.

In an embodiment, the protease inhibitor includes at least one of amperanir, atazanavir sulfate, fosamprenavir calcium, indinavir, lopinavir, ritonavir, nelfinavir, saquinavar mesylate, or saquinavir. In an embodiment, the at least one viral-replication modulator includes at least one of a neuraminidase inhibitor, or amantane derivative. In an embodiment, the at least one neuraminidase inhibitor includes zanamivir, peramivir, or oseltamivir. In an embodiment, the at least one amantane derivative includes amantadine or rimantadine. In an embodiment, the at least one viral-replication modulator includes at least one M2 inhibitor.

In an embodiment, the receptor antagonist includes at least one of a cytokine or chemokine receptor antagonist. In an embodiment, the receptor antagonist includes at least one of a CCR1 receptor antagonist, CCR4 receptor antagonist, CCR5 receptor antagonist, CXCR3 receptor antagonist, CCR3 receptor antagonist, CCR2 receptor, CX3CR1 receptor antagonist, CXCR4 receptor antagonist, or CD4 receptor antagonist. In an embodiment, the CCR5 receptor antagonist includes at least one of TAK-220, or TAK-652. See, for example, U.S. Pat. No. 6,627,651, which is incorporated herein by reference.

In an embodiment, the therapeutic composition includes at least one viral-replication modulator including at least one integrase strand transfer inhibitor. In an embodiment, the at least one integrase strand transfer inhibitor includes at least one of naphthyridine carboxamide, or diketo acid derivative. In an embodiment, the at least one integrase strand transfer inhibitor includes at least one of RSD1624, RSD1625, RSD1996, RSD1997, RSD2196, RSD2197, L-870812, or L-731,988. See, for example, Terrazas-Aranda, et al., Antimicrob. Agents Chemo. vol. 52, no. 7, pp. 2544-2554 (2008), which is incorporated herein by reference. In an embodiment, the at least one integrase inhibitor includes at least one bicyclic pyrazole, including published examples. See, for example, U.S. Pat. No. 7,476,666, which is incorporated herein by reference.

In an embodiment, at least one of the at least one virus entry inhibitor or the viral-replication modulator includes at least one alkyl-urea compound. Several published studies have demonstrated anti-viral efficacy of alkyl-urea compounds against, for example, polio virus, coxsackie virus, HIV, and hepatitis. See, for example, U.S. Pat. No. 4,880,836, which is incorporated herein by reference. Without wishing to be bound by any particular mechanistic theory, alkyl-urea compounds are hydrophobic compounds capable of binding to glycoproteins on the host cell surface, or binding directly to virus particles, thereby reducing the ability of the virus to bind to the host cell. Id. In an embodiment, the therapeutic composition includes, but is not limited to, at least one alkyl-urea compound. In an embodiment, the alkyl-urea compound includes, but is not limited to, at least one monosubstituted lower alkyl compound. In an embodiment, the alkyl-urea compound includes, but is not limited to, butylurea. Id. In an embodiment, the alkyl-urea compound has the formula R—NH—CO—NH₂, wherein R is lower alkyl having 1 to 8 carbon atoms. Id. In an embodiment, R includes at least one methyl, ethyl, propyl, isopropyl, butyl, isobutyl, tertiary butyl, pentyl, hexyl, heptyl, or octyl group. Id.

In an embodiment, the therapeutic composition further comprises at least one immunomodulator formulated to modulate at least one immunological activity. In an embodiment, the therapeutic composition includes at least one immunomodulator. Immunomodulators include activators and inhibitors. Modulating can increase or decrease an immunological activity. Modulating a response includes altering the response by way of e.g., proteins that bind activators or inhibitors, receptors, genetically modified versions of naturally-occurring ligands or receptors, or other molecules that alter the activity of specific molecules.

In an embodiment, the at least one immunomodulator is formulated to increase or decrease at least one immunological activity. In an embodiment, the at least one immunological activity includes, but is not limited to, at least one of inflammation, cell proliferation, cell differentiation, cytokine production, chemokine production, cell transcription, cell translation, receptor binding, receptor activation, receptor rearrangement, intercellular signaling, intracellular signaling, adhesion molecule production or adhesion molecule binding.

In an embodiment, the at least one immunomodulator includes, but is not limited to, at least one of a steroidal or non-steroidal anti-inflammatory agent. In an embodiment, the steroidal anti-inflammatory agent includes, but is not limited to, at least one glucocorticoid. In an embodiment, the steroidal anti-inflammatory agent includes, but is not limited to, at least one of mometasone, amcinonide, desonide, flucinonide, flucinolone acetonide, halcinonide, fluocortolone, prednicarbate, fluprednidene, flunisolide, triamcinoline, triamcinoline acetonide, beclomethasone diproprionate, betamethasone, diproprionate, hydrocortisone, cortisone, tixocortol pivalate, dexamethasone, budesonide, prednisone, methyl prednisolone, or prednisolone. In an embodiment, the non-steroidal anti-inflammatory agent includes, but is not limited to, at least one of gamma linolenic acid, aspirin, ibuprofen, naproxen, carprofen, deracoxib, flurbiprofen, fenoprofen, naburnetone, ketoprofen, piroxicam, indomethacin, tolmetin, etodolac, meclofanamate sodium, mefenamic acid, ketorolac tromethamine, diclofenac, oxaprozin, bromfenac sodium, rofecoxib, suprofen, fenbruprofen, fluprofen, thalidomide, or acetaminophen.

In an embodiment, the at least one immunomodulator is formulated to decrease local or chronic inflammation in the subject. In an embodiment, the therapeutic composition includes, but is not limited to, at least one anti-inflammatory agent formulated to inhibit viral replication. See, for example, U.S. Patent Application Publication No. 20030138399, which is incorporated herein by reference.

In an embodiment, a therapeutic composition as described herein is formulated to modulate the production or activity of at least one cytokine. In an embodiment, a therapeutic composition as described herein is formulated to inhibit or antagonize the production or activity of at least one cytokine. In an embodiment, a therapeutic composition modulates the production or activity of one or more of IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36, IL-37, IL-38, IL-39, IL-40, IL-41, IL-42, IFN-γ, IFN-α, IFN-β, or TNF-α.

Chemokines are biochemical signaling molecules that act to attract other particular molecules, including but not limited to cells, to a specific site. In an embodiment, a therapeutic composition described herein is formulated to modulate the production or activity of one or more chemokines. In an embodiment, a therapeutic composition is formulated to inhibit or antagonize the production or activity of one or more chemokines. In an embodiment, the one or more chemokines include at least one of a CC chemokine, CXC chemokine, C chemokine, or CX3C chemokine. In an embodiment, the one or more chemokines include at least one of CCL1, CCL2, CCL3, CCL4, CCL5, CCL6, CCL7, CCL8, CCL9/CCL10, CCL11, CCL12, CCL13, CCL14, CCL15, CCL16, CCL17, CCL18, CCL19, CCL20, CCL21, CCL22, CCL23, CCL24, CCL25, CCL26, CCL27, CCL28, CCL29, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCL17, CXCL18, CXCL19, CXCL20, CXCL21, CXCL22, XCL1, XCL2, XCL3, XCL4, XCL5, CX3CL1, CX3CL2, CX3CL3.

In an embodiment, the therapeutic composition includes, but is not limited to, at least one microbicide. In an embodiment, the at least one microbicide includes, but is not limited to, at least one of a gp120 peptide, gp41 peptide, nonoxynol-9, cellulose sulfate, or hemolysin A peptide. In an embodiment, the at least one microbicide includes, but is not limited to, one or more quinolone. In an embodiment, wherein the quinolone includes 6-(3-chloro-2-fluorobenzyl)-1-[(2S)-1-hydroxy-3-methylbutan-2-YL]-7-methoxy-4-oxo-1,4-dihydroquinoline-3-carboxylic acid, or salt thereof. See, for example, U.S. Patent Application Publication No. 20040157859, and U.S. Patent Application. Publication No. 20090018162, each of which is incorporated herein by reference.

Any of the therapeutic compositions described herein include formulations for administration to at least one subject. In an embodiment, a therapeutic composition includes a time-release formulation. In an embodiment, the at least one virus entry inhibitor is present in the therapeutic composition in an immediate-release formulation. In an embodiment, the at least one viral-replication modulator is present in the therapeutic composition in a sustained release or time-release formulation. In this regard, the virus entry modulator (e.g., inhibitor) is formulated to reach a maximum fluid concentration quickly following administration to at least one cell, biological tissue, or subject, whereas the viral-replication modulator (e.g., protease inhibitor, reverse transcriptase inhibitor, etc.) is formulated to reach a maximum fluid intake quickly following administration, gradually following administration, or much later following administration, depending on the factors related to the design of the therapeutic composition. Several such factors are disclosed herein throughout the application. In an embodiment, the therapeutic composition includes a greater concentration of the at least one virus entry inhibitor relative to the at least one viral-replication modulator. As discussed herein, in an embodiment, the virus entry inhibitor reaches a maximum concentration in a biological fluid approximately prior to the viral-replication modulator in the therapeutic composition. In an embodiment, the therapeutic composition includes a greater bioeffective concentration of the at least one virus entry inhibitor relative to the at least one viral-replication modulator.

In an embodiment, the at least one viral-replication modulator is present at a weight or volume concentration of about 0.1%, about 0.5%, about 0.8%, about 1.0%, about 2.0%, about 3.0%, about 4.0%, about 5.0%, about 6.0%, about 10.0%, about 15.0%, about 25.0%, about 50.0%, about 75.0%, about 80.0%, about 90.0%, about 95.0%, about 99.0%, about 100.0%, or any value less than or therebetween of the weight or volume concentration respectively, of the at least one virus entry inhibitor in the therapeutic composition.

In an embodiment, the at least one virus entry inhibitor reaches its maximum biological fluid concentration level within approximately 1 minute, approximately 5 minutes, approximately 10 minutes, approximately 20 minutes, approximately 30 minutes, approximately 45 minutes, approximately 1 hour, approximately 2 hours, approximately 5 hours, or any value less than or therebetween.

In an embodiment, the at least one viral-replication modulator reaches its maximum biological fluid concentration level within approximately 20 minutes, approximately 30 minutes, approximately 1 hour, approximately 2 hours, approximately 5 hours, approximately 10 hours, approximately 12 hours, or any value therebetween or greater.

In an embodiment, a therapeutic composition includes at least one solid, liquid, or gas. In an embodiment, a therapeutic composition includes at least one of a suspension, mixture, solution, sol, clathrate, colloid, emulsion, microemulsion, aerosol, ointment, capsule, powder, granule, tablet, suppository, cream, device, paste, resin, liniment, lotion, ampule, elixir, spray, syrup, tincture, detection material, polymer, biopolymer, buffer, adjuvant, diluent, lubricant, disintegration agent, suspending agent, solvent, light-emitting agent, colorimetric agent, glidant, anti-adherent, anti-static agent, surfactant, plasticizer, emulsifying agent, flavor, gum, sweetener, coating, binder, filler, compression aid, encapsulation aid, preservative, granulation agent, spheronization agent, stabilizer, adhesive, pigment, sorbent, nanoparticle, microparticle, piloxymer, nanotube, or gel.

The formulation of any of the therapeutic compositions described herein may be formulated neat or may be combined with one or more acceptable carriers, diluents, excipients, and/or vehicles such as, for example, binders, fillers, tablet disintegrants, flow regulators, plasticizers, wetting agents, dispersants, emulsifiers, solvents, sustained release agents, immediate release agents, antioxidants, propellant gases, buffers, surfactants, preservatives, solubilizing agents, isotonicity agents, and stablilizing agents as appropriate. The therapeutic composition formulated in such a manner typically contains from about 0.1% to about 90% or more by weight of the active agent.

A pharmaceutically acceptable carrier, for example, may be approved by a regulatory agency of the state and/or Federal government such as, for example, the United States Food and Drug Administration (US FDA) or listed in the U.S. Pharmacopeia or other generally recognized pharmacopeia for use in animals, and more particularly in humans. Conventional formulation techniques generally known to practitioners are described in Remington: The Science and Practice of Pharmacy, 21^{St} Edition, Lippincott Williams & Wilkins, which is herein incorporated by reference.

Acceptable pharmaceutical carriers include, but are not limited to, the following: sugars, such as lactose, glucose and sucrose; starches, such as corn starch and potato starch; cellulose, and its derivatives, such as sodium carboxymethyl cellulose, ethyl cellulose, cellulose acetate, and hydroxymethylcellulose; polyvinylpyrrolidone; cyclodextrin and amylose; powdered tragacanth; malt; gelatin, agar and pectin; talc; oils, such as mineral oil, polyhydroxyethoxylated castor oil, peanut oil, cottonseed oil, safflower oil, sesame oil, olive oil, corn oil and soybean oil; polysaccharides, such as alginic acid and acacia; fatty acids and fatty acid derivatives, such as stearic acid, magnesium and sodium stearate, fatty acid amines, pentaerythritol fatty acid esters; and fatty acid monoglycerides and diglycerides; glycols, such as propylene glycol; polyols, such as glycerin, sorbitol, mannitol and polyethylene glycol; esters, such as ethyl oleate and ethyl laurate; buffering agents, such as magnesium hydroxide, aluminum hydroxide and sodium benzoate/benzoic acid; water; isotonic saline; Ringer's solution; ethyl alcohol; phosphate buffer solutions; lactose; microcrystalline cellulose; starch; silicon dioxide; gelatin; sucrose; povidone; hydroxylproply methylcellulose; ethylcellulose; shellac or other glazes; other non-toxic compatible substances employed in pharmaceutical compositions. The pharmaceutical compositions are generally formulated as sterile, substantially isotonic and in full compliance with all Good Manufacturing Practice (GMP) regulations of the U.S. Food and Drug Administration.

In an embodiment, the therapeutic composition includes at least one pharmaceutically-acceptable carrier, inactive ingredient, or excipient, such as, for example, antimicrobial agents, buffers, antioxidants, tonicity agents, and or cryoprotectants and lyoprotectants. Antimicrobial agents in bacteriostatic or fungistatic concentrations can be added to preparations of multiple dose preparations to prevent possible microbial growth inadvertently introduced during withdrawal of a portion of the vial contents. Common examples of antimicrobial agents include phenylmercuric nitrate, thimerosal, benzethonium chloride, benzalkonium chloride, phenol, cresol, and or chlorobutanol. Buffers are used to stabilize a solution against chemical or physical degradation. Common acid salts used as buffers include citrates, acetates and phosphates.

Antioxidants are used to preserve products against oxidation. Common examples of antioxidants include sodium bisulfate, ascorbic acid, and salts, thereof. Tonicity agents are used to ensure that injected material is isotonic with physiological fluids. Common examples of tonicity agents include electrolytes and monosaccharides or disaccharides. Cryoprotectants and lyoprotectants are additives that protect active ingredients from damage due to the freeze-drying process. Common cryoprotectant and lyoprotectant agents include sugars, amino acids, polymers, and polyols.

In addition, any of the therapeutic compositions described herein may include one or more pharmaceutically acceptable salts. Such salts can be prepared from pharmaceutically acceptable non-toxic bases including organic bases or inorganic bases. Salts derived from inorganic bases include sodium, potassium, lithium, ammonium, calcium, magnesium, and the like. Salts derived from pharmaectuically acceptable organic non-toxic bases include salts of primary, secondary, and tertiary amines, basic amino acids, and the like. Some salts include, but are not limited to, sodium phosphate, sodium acetate, sodium bicarbonate, sodium sulfate, sodium pyruvate, potassium phosphate, potassium acetate, potassium biocarbonate, potassium sulfate, potassium pyruvate, disodium DL-α-glycerol-phosphate, or disodium glucose-6-phosphate. Phosphate salts of sodium or potassium can include, for example, monobasic form, dibasic form, or a mixture thereof.

Salt crystals may be hydrated, for example, when dissolved in an aqueous solution at a certain molar concentration, are equivalent to the corresponding anhydrous salt dissolved in an aqueous solution at the same molar concentration.

The therapeutic compositions described herein may include an admixture with one or more pharmaceutically acceptable excipients. For example, the therapeutic compositions described herein can be associated with chemical moieties which may improve particular properties, (i.e., the composition's solubility, absorption, biological half life, etc.), or decrease other properties (i.e., toxicity, undesirable side effects, etc.) Some non-limiting examples of moieties capable of mediating such effects are disclosed in Remington's Pharmaceutical Sciences, 17^{th} Edition, A. R. Gennaro, ed., Mack Publishing Co., Easton, Pa. (1995). Procedures for coupling such moieties to a molecule are well known in the art and can be performed by standard techniques.

As described herein, in order to maximize the efficacy of the therapeutic composition, the composition includes an overall release profile such that when administered the virus entry inhibitor component is formulated to reach a maximum biological fluid concentration by at least one time point approximately prior to a time point at which the viral-replication modulator eaches a maximum biological fluid concentration.

In an embodiment, the therapeutic composition is formulated for oral administration. In an embodiment, the viral-replication modulator component of the oral formulation is a delayed release dosage form. In an embodiment, the delayed release dosage form includes a pH sensitive delayed dosage form. In an embodiment, the delayed release dosage form includes a non-pH sensitive delayed release dosage form.

In an embodiment, the therapeutic composition comprises multiple viral-replication modulator components. In an embodiment, at least two of the multiple viral-replication modular components have a similar release profile. In an embodiment, none of the multiple viral-replication modular components have similar release profiles.

In an embodiment, the therapeutic composition comprises multiple virus entry inhibitor components. In an embodiment, at least two of the multiple virus entry inhibitor components have a similar release profile. In an embodiment, none of the multiple virus entry inhibitor components have similar release profiles. Regardless of the number of components in the therapeutic composition, the at least one virus entry inhibitor component(s) are formulated to reach a maximum biological fluid concentration approximately prior to a time point at which the viral-replication modulator component(s) reach a maximum biological fluid concentration.

In an embodiment, the virus entry inhibitor reaches a maximum concentration in a biological fluid (e.g., blood serum) after initiation of release, within about 0.5 hours, about 1.0 hour, about 2.0 hours, about 3.0 hours, about 4.0 hours, about 5.0 hours, about 6.0 hours, or any value less than or therebetween.

In an embodiment, the viral-replication modulator reaches a maximum concentration in a biological fluid (e.g., blood serum) after initiation of release, within about 1.0 hour, about 2.0 hours, about 3.0 hours, about 4.0 hours, about 5.0 hours, about 6.0 hours, about 8.0 hours, about 10.0 hours, or any value less than or therebetween.

In an embodiment, the therapeutic composition is foimulated for a single administration in about a 24 hour period. In an embodiment, the therapeutic composition is formulated for a single administration in about a 12 hour period. In an embodiment, the therapeutic composition is formulated for a single administration in about a 6 hour period.

In an embodiment, the therapeutic composition includes an oil-in-water emulsion, or a water-in-oil emulsion. In this formulation, the immediate release dosage form is in the continuous phase, and the delayed release dosage form is in a discontinuous phase.

Design and development of specific formulations are standard practice in the art. For example, formulation development and optimization involves varying excipient levels, processing methods, identifying discriminating dissolution methods, and subsequent scale-up of the final product, all of which are standard practices in the art. See, for example, Drug Del. Vol. 3, no. 4 (2003) on the worldwide web at: drugdeliverytech.com/ME2/Segments/Publications, the content of which is incorporated herein by reference. Thus, in an embodiment, multiple components are formulated into a single composition with varying rates of release. See, for example, U.S. Pat. No. 7,074,417, which is incorporated herein by reference.

For example, published studies have reported inhibition of viral entry by varicella zoster virus by mannose 6-phosphate and heparin. See, for example, Zhu, et al., PNAS, vol. 92, pp. 3546-3550 (1995), which is incorporated herein by reference. For example, polyanionic substances including but not limited to a compound containing at least one of heparin, suramin, pentosan sulfate, PI-88, DL-galactan hybrid, sulfated alpha-D-glucan, carrageenan, sulfated galactomannan, or polyoxotungstate, can block interaction of virus with heparain sulfate. Id. Other examples of agents that can be used to inhibit viral fusion include, but are not limited to, amantadine, rimantadine, chlorpromazine, synthetic peptides, or tetracycline derivatives. Id.

In an embodiment, the virus entry inhibitor includes an immediate release tablet, and the viral-replication modulator includes a delayed release tablet, such that the virus entry inhibitor reaches a maximum biological fluid (e.g., blood serum) concentration by at least one time point approximately prior to the time point at which the viral-replication modulator reaches a maximum biological fluid concentration.

In an embodiment, the at least one virus entry inhibitor or viral-replication modulator includes one or more peptides or peptide mimetics. In an embodiment, the one or more peptides or peptide mimetics are used to inhibit at least viral entry or fusion with the host cell membrane or one or more membrane components, or to inhibit function or structure of the interaction of the virus and host cell membrane or components thereof. For example, the HIV fusion inhibitor enfuvirtide is a peptide mimetic that interferes with gp41 mediated cell fusion. It has been reported that peptides or peptide mimetics inhibit the infectivity of viruses, including but not limited to Dengue virus, West Nile virus, Herpesvirus, and Ebola virus. See, for example, Hrobowski, et al., Virology J. vol. 2, no. 49 (2005); Okazaki & Kida. J. Gen. Virol. vol. 85, pp. 2131-2137 (2004); and Watanabe et al., J. Virol., vol. 174, pp. 10194-10201 (2000), each of which is incorporated herein by reference. In an embodiment, the peptide may be all or part of the ligand that normally binds the host cell receptor. For example, peptides containing viral integrin ligand sequences (CPRP and RDGEE) attenuate rotavirus infection of cells in vitro. See, for example, Graham, et al., J. Virol., vol. 77, pp. 9969-9978 (2003), which is incorporated herein by reference.

In an embodiment, at least one of the virus entry inhibitor or viral-replication modulator may be a soluble version of the host cell receptor that normally recognizes and binds to the virus. Soluble receptors bind up the viral particles and prevent the virus from binding/interacting with endogenous cellular receptors and as such prevent infection. For example, soluble forms of the coxsackievirus-adenovirus receptor (CAR) attenuates infectivity of Coxsackie B virus See, for example, Goodfellow, et al., J. Virol., vol. 79, pp. 12016-12024 (2005), which is incorporated herein by reference. It has been reported that recombinant soluble low density lipoprotein receptor fragments inhibit minor group rhinovirus infection in vitro. See, for example, Marlovits, et al., FASEB J. vol. 12, pp. 695-703 (1998), which is incorporated herein by reference.

Behavioral and biological risk factors are associated with the risk of transmission of any virus, including close physical contact among humans or with other animal species, including contact with bodily fluids (e.g., by way of coughing, sneezing, sexual contact, etc.). For example, for HIV transmission, risk factors include frequency and types of sexual contact, use or nonuse of condoms, immunologic status, presence or absence of AIDS, male circumcision, level of plasma HIV-1 RNA levels, presence or absence of chemokine receptors, or presence or absence of other sexually transmitted diseases. See, for example, Quinn, et al., New Engl. J. Med., vol. 342, no. 13, pp. 921-929 (2009), which is incorporated herein by reference.

In an embodiment, a method for administering at least one therapeutic composition to a subject (including a pregnant subject) includes, but is not limited to, at least one formulation for administration to a subject by at least one route including one or more of oral, topical, transdermal, epidermal, intravenous, intraocular, tracheal, transmucosal, intracavity, subcutaneous, intramuscular, inhalation, fetal, intrauterine, placental, interdermal, intradermal, enteral, parenteral, surgical, or injection. In an embodiment, the therapeutic composition is formulated for a single oral dose. In an embodiment, the therapeutic composition is formulated for administration of a single fetal injection dose.

In an embodiment, the at least one subject includes one or more of a vertebrate or invertebrate, insect cells, insects, bacteria, algae, plankton, or protozoa. In an embodiment, the at least one subject includes one or more of a reptile, mammal, amphibian, bird, or fish. In an embodiment, the at least one subject includes at least one human. In an embodiment, the at least one subject includes at least one of livestock, pet, zoo animal, undomesticated herd animal, wild animal, or product animal.

In an embodiment, the at least one subject includes at least one of a sheep, goat, frog, dog, cat, rat, mouse, vermin, monkey, duck, horse, cow, pig, chicken, shellfish, fish, turkey, llama, alpaca, bison, buffalo, ape, primate, ferret, wolf, fox, coyote, deer, rabbit, guinea pig, yak, chinchilla, mink, reindeer, elk, camel, fox, elk, deer, raccoon, donkey, or mule.

In an embodiment, the subject includes at least one donor or recipient. In an embodiment, the donor includes at least one cadaver.

In an embodiment, the therapeutic composition is formulated for use in inhalation administration, for example, by coating particles and optionally micronizing the particles for greater uptake by inhalation. In an embodiment, the therapeutic composition is formulated for oral administration in the form of a pellet, capsule, tablet, particle, or liquid suspension. In an embodiment, each of the dosage forms of the therapeutic composition are formulated as a tablet, with each of the tablets put into a capsule to form a unitary composition.

In an embodiment, intracavity route of administration includes, but is not limited to, at least one of buccal, oral, vaginal, uterine, rectal, nasal, peritoneal, ventricular, or intestinal. In an embodiment, the route of administration includes embryonic or fetal injection. In certain instances, maternal to fetal transmission of virus occurs later during the gestational term, or during the birthing process. See, for example, the worldwide web at: AIDSinfo.nih.gov/contentFiles/GIChunks/peri_{—}12.pdf, the content of which is incorporated herein by reference. In an embodiment, administration of the at least one anti-viral therapeutic composition occurs by abdominal injection. In an embodiment, the abdominal injection includes, but is not limited to, intrauterine injection. In an embodiment, the administration includes, but is not limited to, embryonic or fetal injection. In an embodiment, the administration is formulated to be administered prior to, during, or subsequent to the birthing process of the at least one offspring subject (e.g., fetus). In an embodiment, the administration is formulated to be administered to a pregnant subject during gestation of at least one offspring subject. In an embodiment, the administration is formulated to be administered prior to delivery of the at least one offspring subject. In an embodiment, the at least one anti-viral therapeutic composition is administered to at least one offspring subject by way of in situ, in vitro, in vivo, in utero, or ex vivo administration.

Some factors that affect transfer of at least one therapeutic composition from the mother to the unborn offspring include, but are not limited to lipid solubility (e.g., lipophilic molecules diffuse more readily than lipophobic molecules), degree of ionization (e.g., non-ionized fraction diffuses more readily), pH of maternal blood (e.g., affects the degree of ionization and depends on the pKa of the therapeutic composition), protein binding of the therapeutic composition (e.g., unbound agent diffuses, acidosis reduces the bound fraction), fetal/maternal concentration gradient, placental blood flow, or molecular weight of the therapeutic composition (compositions with molecular weight of less than or approximately equal to 600 Da more readily diffuse).

In an embodiment, a method for administering at least one anti-viral therapeutic composition further includes, but is not limited to, at least one of counseling or testing of a pregnant subject, Cesarean delivery of the at least one offspring subject, or avoidance of breastfeeding. See, for example, the worldwide web at: netwellness.org/healthtopics/aidshiv/mothertochild.cfm, the content of which is incorporated herein by reference.

In an embodiment, at least one therapeutic composition disclosed herein is administered to a pregnant subject who is believed to be at risk of contracting at least one virus (e.g., HIV, hepatitis, etc.) or who already harbors at least one virus and may or may not show any symptoms or signs of related disease. In an embodiment, the viral status of the pregnant subject is unknown or unknowable. In an embodiment, the at least one offspring subject of the pregnant subject (e.g., embryo, fetus, etc.) is believed to be at risk of contracting at least one virus (e.g., HIV, hepatitis, etc.) or already harbors at least one virus. In an embodiment, the at least one offspring subject of the pregnant subject is believed to be at risk of contracting at least one virus from its mother subject. For example, mother-to-child transmission of HIV is significantly reduced following postnatal administration of anti-retroviral dugs to infants born to HIV-infected mothers who received no antenatal or antepartum prophylaxis. See, for example, Omrani and Freedman, Brit. Med. Bull., vols. 73-74, pp. 93-105 (2005), which is incorporated herein by reference.

In an embodiment, a method includes, but is not limited to, administering at least one anti-viral therapeutic composition to the at least one offspring subject subsequent to birth. In an embodiment, a method includes, but is not limited to, administering at least one anti-viral therapeutic composition to the at least one offspring subject until the viral status of the at least one offspring subject is determined. In certain instances, the at least one offspring subject is tested for at least one virus (e.g., HIV, hepatitis, etc.) on the standard schedule of about birth to about 14 days, about 1 to about 2 months, or about 3 to about 6 months, or any other schedule. Id. In an embodiment, the at least one offspring subject born to a mother known to be infected with at least one virus (e.g., HIV, hepatitis, etc.) begins or continues direct administration of at least one anti-viral therapeutic composition at the time of birth, or shortly thereafter. For example, standard protocols begin preventative treatment of infants born to HIV-positive mothers typically within about 6 to 12 hours after birth. Id.

Published studies have shown that placental transfer to the amniotic fluid occurs for nucleoside analogue reverse transcriptase inhibitors (NRTIs). See, for example, Chappuy, et al., Antimicrob. Agents & Chemo. vol. 48, no. 11, pp. 4332-4336 (2004), which is incorporated herein by reference. Additionally, published studies have shown that protease inhibitors also cross the placenta, but at lower levels than the NRTIs. See, Marzolini, et al., AIDS, vol. 16, pp. 889-893 (2002), which is incorporated herein by reference.

Published studies indicate that direct administration of an agent to at least one offspring during gestation results in delivery of the agent, and corresponding clinical results. For example, fetal tachyarrhythmias can be treated, for example, by direct injection of digoxin, propranalol, verapamil, amiodarone, or procainamide by intramuscular, intravenous, or intraperitoneal injection. See, for example, De Cane et al., Abstract, Prenat. Diagn. vol. 14, no. 8, pp. 762-765 (1994), which is incorporated herein by reference.

Further published studies indicate that treatment of cytomegalovirus (CMV)-infected fetuses by administration of CMV hyperimmunoglobulin into the fetal abdominal cavity reduced clinical manifestations of CMV disease. See, for example, Negishi, et al., Abstract, J. Perinatol. vol. 6, pp. 466-469 (1998); and Sato, et al., Abstract, J. Obs. Gyn. Res. vol. 33, no. 5, pp. 718-721 (2007), each of which is incorporated herein by reference.

Generally, virus families include a group of genetically-related forms of HIV. Clades of virus families are also called genetic subtypes and generally have geographic distribution patterns. For example, clades A, C, and D are most common in Africa, while Glade B HIV is most commonly found in North America and Europe. See, for example, Hu, Abstract, Int. Conf. AIDS, vol. 11, no. 40, We.C.451, (1996), which is incorporated herein by reference.

Several classes of HIV-1 have developed across the globe, including but not limited to, M (major), O (outlying), and N (new). The M group accounts for about 90% of reported HIV/AIDS cases, and viral envelopes of this group are diverse, such that the virus has been subclassified into at least nine major clades, including but not limited to A-D, F-H, J, K, and several circulating recombinant forms. Clades may show differences in co-receptor usage for infection (e.g, CXCR4, CCR5, etc.) and syncytia-inducing capacity. See, for example Stebbing and Moyle, AIDS Rev. vol. 5, pp. 205-213 (2003), which is incorporated herein by reference. In some cases, certain clades respond better to single therapeutic treatments, or develop resistance to a particular single treatment. In an embodiment, genetic or proteomic information is obtained from the virus common in the subject's locale, or present in a biological fluid or tissue of the subject, prior to or in conjunction with determining a treatment regimen, or administration protocol.

The delivery may include inhalation, depot injections, implants, or other mode of delivery by way of a device. In an embodiment, the oral formulation includes at least one of a cavity, layer, or coating that contains at least part of the immediate release component. In an embodiment, the therapeutic composition is formulated for administration of a single dose. In an embodiment, the administration of a single dose includes a single oral dose. In an embodiment, the administration of a single dose includes a single intrauterine dose. In an embodiment, the administration of a single dose includes a single fetal injection dose.

In an embodiment, a method comprises administering to an asymptomatic subject infected or at risk of infection with at least one virus, an effective amount of a therapeutic composition; the therapeutic composition including an effective amount of at least one virus entry inhibitor in a first formulation, an effective amount of at least one viral-replication modulator in a second formulation, and at least one pharmaceutically acceptable carrier or excipient; wherein the first formulation regulates the release of the at least one virus entry inhibitor and the second formulation regulates the release of the at least one viral-replication modulator; and wherein the maximum concentration in a biological fluid of the at least one virus entry inhibitor occurs at a time point approximately prior to the maximum concentration of the at least one viral-replication modulator.

In an embodiment, the method comprises administering to a biological tissue infected or at risk of infection with at least one virus, an effective amount of a therapeutic composition; the therapeutic composition including an effective amount of at least one virus entry inhibitor in a first formulation; an effective amount of at least one viral-replication modulator in a second formulation; and at least one pharmaceutically-acceptable carrier or excipient; wherein the first formulation regulates the release of the at least one virus entry inhibitor and the second formulation regulates the release of the at least one viral-replication modulator; and wherein the maximum concentration in a biological fluid of the at least one virus entry inhibitor occurs at a time point approximately prior to the maximum concentration of the at least one viral-replication modulator.

In an embodiment, the biological fluid includes at least one of water, urine, mucus, breast milk, tears, sweat, ascites, fecal fluid, blood, blood serum, saliva, gastrointestinal fluid, vaginal fluid, lymph, saline, or any fluid component thereof. In an embodiment, the biological fluid includes blood serum. In an embodiment, the at least one biological fluid is located at least one of in situ, in vitro, in vivo, in utero, in planta, in silico, or ex vivo.

In an embodiment, a method includes administering at least one therapeutic composition described herein to at least one biological tissue of an asymptomatic subject.

In an embodiment, the at least one biological fluid is located in at least one subject. In an embodiment, the at least one biological fluid or tissue includes is located in at least one donor or recipient. In an embodiment, the at least one donor includes at least one cadaver.

In an embodiment, the at least one biological tissue of the subject includes at least one of skin, brain, lung, liver, spleen, bone marrow, thymus, heart, myocardium, endocardium, pericardium, lymph node, blood, bone, cartilage, pancreas, kidney, gall bladder, stomach, intestine, testis, uterus, rectum, nervous system, eye, scalp, nail bed, ear, ovary, oviduct, tongue, tonsil, adenoid, liver, blood vessel, lymph, lymph node, breast, bladder, urethra, ureter, prostate, vas deferens, fallopian tubes, esophagus, oral cavity, nasal cavity, otic cavity, connective tissue, muscle tissue, or adipose tissue.

In an embodiment, the asymptomatic subject infected or at risk of infection includes an asymptomatic subject who has potentially been exposed to the at least one virus but remains asymptomatic. In an embodiment, the asymptomatic subject infected or at risk of infection includes an asymptomatic subject who has knowingly been exposed to the at least one virus but who remains asymptomatic. In an embodiment, the asymptomatic subject infected or at risk of infection includes an asymptomatic subject who has potentially been infected with the at least one virus but who remains asymptomatic. In an embodiment, the asymptomatic subject infected or at risk of infection includes an asymptomatic subject who has knowingly been infected with the at least one virus but who remains asymptomatic. In an embodiment, the therapeutic composition is administered to an asymptomatic subject prior to, during, or subsequent to a specific act that potentially exposes the asymptomatic subject to virus infection. In an embodiment, the specific act is statistically likely to cause virus infection. In an embodiment, the specific act has been shown to cause virus infection in subjects who engage in the specific act. For example, in an embodiment the specific act includes, but not be limited to intravenous drug use (e.g., using a needle or other apparatus that can spread virus infection), body piercing (e.g., using a needle or other apparatus that can spread virus infection), engaging in sexual conduct (e.g., with another subject known or suspected of being infected with a virus), contacting at least one bodily fluid (e.g., a bodily fluid from a subject known or suspected of being infected with a virus), contacting aerosolized or particulate biological fluid, inhaling environmental contamination (e.g., in hospitals, child care or elder care facilities, feedlots or other agricultural environments, or other industrial worksites), or handling biological tissues or fluids (e.g., biological tissues or fluids known or suspected of being infected with a virus).

In an embodiment, the method further comprises testing the subject for at least one of exposure or infection with at least one virus. In an embodiment, the method further comprises altering the therapeutic composition for the subject with a positive test for at least one of exposure or infection with at least one virus. In an embodiment, the testing includes at least one assay. In an embodiment, the assay includes at least one technique that includes spectroscopy, microscopy, electrochemical detection, polynucleotide detection, histological examination, biopsy analysis, fluorescence resonance energy transfer, electron transfer, enzyme assay, electrical conductivity, isoelectric focusing, chromatography, immunoprecipitation, immunoseparation, aptamer binding, filtration, electrophoresis, immunoassay, or radioactive assay.

In an embodiment, administering includes a single administration in an approximately twenty four hour period. In an embodiment, administering includes at least two or more administrations in an approximately twenty four hour period.

In an embodiment, administering includes administering the therapeutic composition prior to a specific act that potentially exposes the asymptomatic subject to a virus infection occurs at least about 7 days, at least about 6 days, at least about 5 days, at least about 4 days, at least about 3 days, at least about 2 days, at least about 1 day, at least about 20 hours, at least about 10 hours, at least about 1 hour, at least about 45 minutes, at least about 30 minutes, at least about 10 minutes, at least about 5 minutes, at least about 1 minute, or any value therebetween or greater prior to the specific act. In an embodiment, administering includes administering the therapeutic composition to the asymptomatic subject within the therapeutically effective dosage time prior to a specific act that potentially exposes the asymptomatic subject to a virus infection. In an embodiment, administering includes administering the therapeutic composition to at least one biological fluid or tissue expected to be exposed to at least one virus.

In an embodiment, a method comprises administering to a biological tissue infected or at risk of infection with at least one virus, an effective amount of a therapeutic composition; the therapeutic composition including at least one virus entry inhibitor, at least one viral-replication modulator, and at least one pharmaceutically acceptable carrier or excipient; wherein the at least one virus entry inhibitor is configured to reach a maximum biological fluid concentration by at least one time point approximately prior to a time point at which the at least one viral-replication modulator reaches a maximum biological fluid concentration.

In an embodiment, the at least one biological tissue is located at least one of in situ, in vitro, in vivo, in utero, in planta, in silico, or ex vivo. In an embodiment, the at least one biological tissue is ingestible, implantable or transplantable.

In an embodiment, a method comprises administering to an unborn offspring subject infected or at risk of infection with at least one virus, an effective amount of a therapeutic composition; the therapeutic composition including at least one virus entry inhibitor, at least one viral-replication modulator; and at least one pharmaceutically-acceptable carrier or excipient; wherein the at least one virus entry inhibitor is formulated to reach a maximum biological fluid concentration by at least one time point approximately prior to a time point at which the at least one viral-replication modulator reaches its maximum biological fluid concentration.

In an embodiment, the asymptomatic subject infected, or at risk of infection, includes an asymptomatic subject who has potentially been exposed to at least one virus. In an embodiment, the asymptomatic subject infected, or at risk of infection, includes an asymptomatic subject who has knowingly been exposed to at least one virus. In an embodiment, the asymptomatic subject infected, or at risk of infection, includes an asymptomatic subject who has potentially been infected with at least one virus. In an embodiment, the asymptomatic subject infected, or at risk of infection, includes an asymptomatic subject who has knowingly been infected with at least one virus.

In an embodiment, a method of administration of a therapeutic composition described herein further comprises testing the subject for infection with the at least one virus. Testing can be done, for example, by assaying at least one biological tissue (e.g., fluid, cells, etc.) by standard techniques, to identify viral infection or viral load. Such standard techniques include but are not limited to, spectroscopy, microscopy, electrochemical detection, polynucleotide detection, histological examination, biopsy analysis, fluorescence resonance energy transfer, electron transfer, enzyme assay, electrical conductivity, isoelectric focusing, chromatography, immunoprecipitation, immunoseparation, aptamer binding, filtration, electrophoresis, immunoassay, or radioactive assay. In an embodiment, the method further comprises altering the therapeutic composition if the testing of the subject confirms infection of the at least one virus. For example, the therapeutic composition, formulation, method of administration, dosing, or other parameter of prophylactic treatment or other treatment (e.g., responsive to virus infection) can be altered in accordance with the test results and overall goal of preventing viral infection or preventing symptoms of viral infection. In an embodiment, the method further comprises altering the therapeutic composition for the subject with a positive test for at least one of exposure or infection with at least one virus. In an embodiment, the method further comprises testing the asymptomatic subject for the level of a previously administered therapeutic composition prior to administration of a second dosage of the same or different therapeutic composition.

Any of the methods disclosed herein may include detecting in the subject, or biological tissue(s), at least one level of at least one biological signaling molecule, or cell (e.g. lymphocytes) that is associated with an immulogical response to a virus, or that is associated with at least one disease or condition related to viral infection.

Detection of one or more biological signaling molecules, or cells, can be conducted by any method known in the art, including but not limited to analyzing one or more biological tissues or fluids from the subject. Analyzing one or more biological fluids can be performed by any of a variety of methods known in the art, including but not limited to utilizing one or more of thin-layer chromatography, mass spectrometry, nuclear magnetic resonance, polymerase chain reaction, reverse transcriptase, Northern blot, Western blot, microscopy, flow cytometry, antibody binding, enzyme-linked immunosorbent assay, radioactive absorption or release, microfluidic analysis, nucleic acid chip array analysis, protein chip array analysis, chemical sensor analysis (including arrays), biosensor analysis, cell counting, or cell sorting.

In an embodiment, the at least one biological signaling molecule, or cell, includes but is not limited to, one or more of a nucleic acid, amino acid, peptide, polypeptide, protein, glycopeptide, glycoprotein, glycolipid, lipopolysaccharide, peptidoglycan, proteoglycan, lipid, metalloprotein, liposome, or carbohydrate. Carbohydrates may include, but not be limited to, oligosaccharides, glycans, glycosaminoglycans, or derivatives thereof.

In an embodiment, the at least one biological signaling molecule, or cell, includes but is not limited to at least one cytokine, chemokine, cellular receptor, intracellular second messenger, protease, kinase, enzyme, cellular receptor ligand, transcription factor, hormone, or white blood cell.

White blood cells include, but are not limited to, neutrophils, eosinophils, basophils, dendritic cells, lymphocytes, monocytes, histiocytes, mast cells, microglia, or macrophages. Lymphocytes include, but are not limited to, T cells, B cells, or natural killer cells.

In an embodiment the one or more biological signaling molecules are detected by one or more recognition molecules specific to the one or more biological signaling molecules. The recognition molecules may include, but not be limited to, an antibody, affibody, DNA-recognition molecule, aptamer (or oligonucleotide), or other molecule. For example, DNA aptamers have been shown to prevent influenza virus infection by blocking the receptor binding region of the viral hemagglutinin. See, for example, Jeon et al., J. Biol. Chem., vol. 279, no. 46, pp. 48410-48419 (2004); and U.S. Patent Application Publication No. 20070059806, each of which is incorporated herein by reference.

In an embodiment, the process of determining an effective aptamer for a particular virus includes standard techniques, such as in vitro selection of specific binding to a target molecule, and isolating the molecule. One such technique, SELEX, or selective evolution of ligands by exponential enrichment, utilizes a pool of oligonucleotides containing a region of randomized nucleotides (generally 30-100 nucleotides in length) flanked by conserved sequences that contain primer-binding sites for use in PCR. Id. The oligonucleotides are bound to the target molecule, and the oligos exhibiting the tightest binding are isolated. Next, the isolated oligos are amplified by PCR, and oligos are selected based on selection criteria. Id.

In some instances, levels of particular biological signaling molecules may be assayed in a bodily fluid or tissue using gas or liquid chromatography with or without mass spectrometry. A bodily fluid may include blood, lymph, saliva, urine, sweat, ascites, serum, urogenital secretion, bone marrow, a tissue secretion or excretion, or other fluid.

A level of one or more biological signaling molecules may also be assayed in a bodily fluid or tissue using a recombinant cell based assay or sensor. A sensor may include, for example a chemical sensor, biosensor, protein array, or microfluidic device.

A delivery regimen may include a therapeutically effective amount of one or more therapeutic compositions described herein that include immunomodulators or immune system component analogs. The regimen may include a schedule of changes in the dosage of the therapeutic composition to maintain a desired level in the subject of one or more molecules related to the therapeutic composition. Such treatment may be individualized for the biological tissue or subject to which the therapeutic composition is administered. Administration of at least one of the therapeutic compositions included herein may prevent or delay the onset of symptoms, complications, or biochemical indicia of a disease or condition associated with viral infection, or alleviate the symptoms, arrest, or inhibit further development of the disease, condition, or disorder associated with viral infection. Administration of at least one therapeutic composition described herein may be prophylactic to prevent or delay the onset of a disease or condition associated with viral infection. Administration of at least one therapeutic composition described herein may prevent the manifestation of clinical or subclinical symptoms thereof, or provide suppression or alleviation of symptoms after the manifestation of the disease associated with viral infection. For example, published reports of post-exposure prophylaxis for HIV has demonstrated resistance to HIV infection up to six months, following exposure to the virus. See, for example, Mechai, et al., J. Med. Virol., vol. 80, pp. 9-10, (2008), which is incorporated herein by reference.

A delivery regimen may be continuous and uninterrupted, which indicates that there is no break in the treatment regimen during the treatment period. Continuous, uninterrupted administration of a combinational therapeutic composition includes that the combination may be administered during the entire treatment period, e.g., at least once daily or on a continuous and uninterrupted basis. The treatment regimen may be given to maintain an in vivo therapeutic level or a determined cyclic level of the one or more agents of the at least one therapeutic composition.

In an embodiment, one or more methods of administration of the at least one therapeutic composition are based on a genetic or proteomic profile of the subject. Medical evaluation regarding genetic profiling or genetic testing can be provided as a current determination of genetic risk factors, or as part of the subject's medical history. Genetic profiling or genetic testing can be used to design a treatment regimen and thus determine an optimal level individualized for the subject. A physician may use the genetic profile or genetic testing information to determine a genetic basis for needed treatment based on baseline or physiological levels of biochemical components.

Prior to or in conjunction with determining an administration regimen, additional information can be obtained regarding any particular inflammatory disease or condition associated with viral infection, in relation to any possible therapeutic treatment derived from population databases. The medical evaluation can include information in a population database on disease risks, available drugs and formulations, and documented population responses to drugs and formulations.

In an embodiment, one or more polymorphisms are determined prior to administration of at least one therapeutic composition described herein, which could allows for such therapeutic composition to be tailored to a particular subject's genetic makeup.

In an embodiment, methods disclosed herein relate to treating a subject afflicted with or suspected of being afflicted with at least one disease or condition associated with viral infection, by administering to the subject an effective amount of a therapeutic composition disclosed herein. Certain aspects of diseases or conditions associated with viral infction include, but are not limited to, an inflammatory condition or disease state at a particular time, including an atypical inflammatory condition for a subject or tissue.

The disease or condition associated with viral infection may be clinically diagnosed disease or the subject may be suspected of being infected by at least one virus based on any exposure events that are likely to increase the risk of infection.

As set forth herein, the therapeutic compositions disclosed are formulated by standard practice. In certain instances, in order to account for bioavailability, a formulation may be provided in rapid release, or extended release form prior to administration. Likewise, liposomes, microsomes, or other vehicles or composition modifications allow for regulating the dosage by increasing or decreasing the rate of therapeutic composition delivery, maintenance, decomposition, clearance, or other factors. For example, one particular component may have bioavailability properties that require it to be modified by standard techniques so that it can be administered simultaneously with another component. Similarly, where multiple compnoents are included in a single composition, it may be necessary to modify one or more of the components by standard techniques.

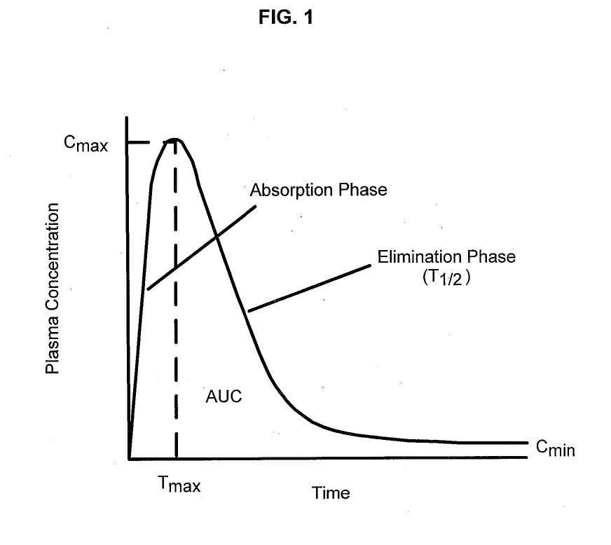

As indicated in FIG. 1, the plasma concentration over time can be determined for any particular therapeutic agent (e.g., component of a therapeutic composition). For example, the maximum blood plasma concentration (Cₘₐₓ) for a particular agent occurs at a time point (Tₘₐₓ), when the area under the curve (AUC) is greatest as the agent appears in the systemic circulation. The integral of blood plasma concentration (AUC) is measured with respect to the time point of administration to the Tₘₐₓ, to the time point at which the lowest amount of agent is observed (Cₘᵢₙ), until no detectable level of the agent remains in the blood plasma.

According to pharmacokinetic standards, the half-life (T_{1/2}) of a therapeutic agent includes the amount of time for the plasma concentration of an agent to fall by 50% when the first-order kinetics are observed. Some therapeutic agents have an initial redistribution phase with a short half-life (T_{1/2a}), followed by an elimination phase with a longer half-life (T_{1/2b}). See, for example, the worldwide web at: frca.co.uk; or saladax.com/pharmacology, the content of each of which is incorporated herein by reference.

Clearance of an agent includes the apparent volume of plasma from which an agent is entirely removed per unit time, and is generally expressed in proportion to body weight of the subject, or surface area. Id. The volume of distribution includes the volume into which an agent appears to be uniformly distributed at the concentration measured in plasma. The volume of distribution can include a steady state volume of distribution equal to the amount of the agent in the subject's body, illustrated by (n) divided by the plasma concentration (C): V_{d}=n/C. The volume of distribution also is equal to the clearance (Cl) times elimination half-life divided by ln 2: V_{d}=(1/ln 2)Cl(T_{1/2b}). Id.

The bioavailability of a particular agent includes the proportion of a dose of a specified agent preparation entering the systemic circulation after administration by a specified route, and is indicated by the AUC. The bioavailability includes “oral bioavailability,” when the agent is administered orally. For example, as illustrated in FIG. 1, the absorption phase for oral administration is indicated by the time required to reach Cₘₐₓ, whereas the elimination phase is indicated by the time required to clear or eliminate the agent from the blood plasma.

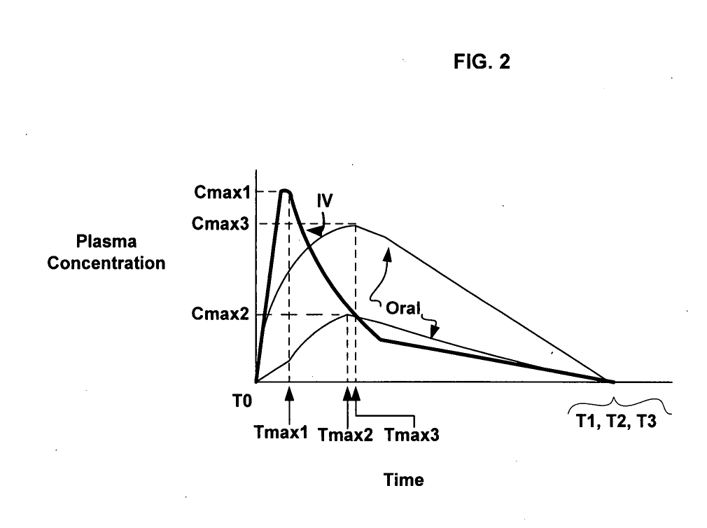

As illustrated in FIG. 2, the maximum blood plasma concentration (Cₘₐₓ₁) for an agent administered intravenously (IV) occurs almost immediately (Tₘₐₓ₁), whereas the maximum blood plasma concentration (Cₘₐₓ₂, Cₘₐₓ₃) for an agent administered orally (Oral) and formulated as an extended release, has an extended absorption phase prior to reaching the maximum blood plasma concentration (Tₘₐₓ₂, Tₘₐₓ₃). Clearance time is calculated from the time of administration of the agent (T0) until no detectable level of the agent remains in the blood plasma (T1, T2, T3). The amount of time each agent or component of the therapeutic composition remains detectable in the biological fluid(s) or biological tissue(s) varies, depending on the specific composition. The illustrated Figures described herein are provided as examples only, and are in no way limiting.

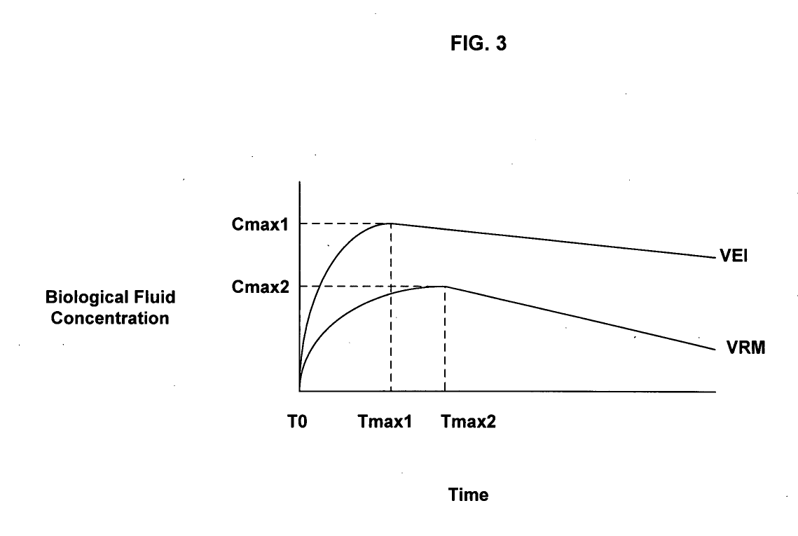

As illustrated in FIG. 3, in one example biological fluid concentration profile, the therapeutic composition includes at least one virus entry inhibitor (VEI) and at least one viral-replication modulator (VRM), wherein the at least one virus entry inhibitor is formulated to reach a maximum biological fluid concentration (Cmax1) by at least one time point (Tmax1) approximately prior to a time point (Tmax2) at which the viral-replication modulator reaches a maximum biological fluid concentration (Cmax2). Time of administration of the therapeutic composition is illustrated as T0, with T1 and T2 indeterminate.

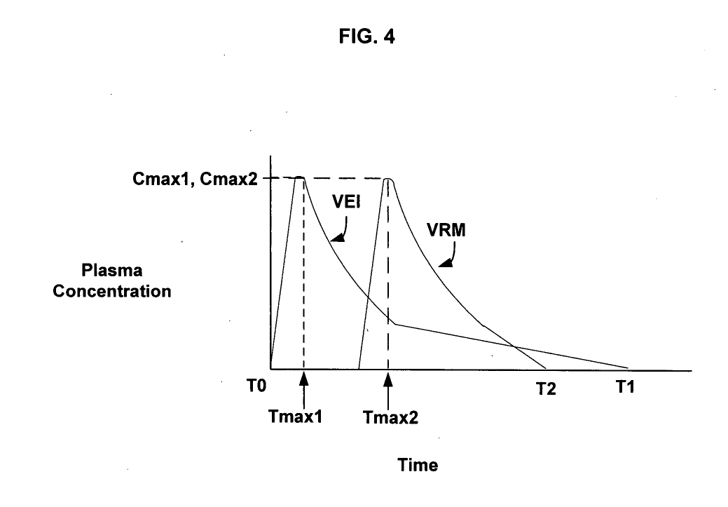

As illustrated in FIG. 4, in one example biological fluid concentration profile, the therapeutic composition includes at least one virus entry inhibitor (VEI) and at least one viral-replication modulator (VRM), wherein the at least one virus entry inhibitor is formulated to reach a maximum biological fluid concentration (Cmax1) by at least one time point (Tmax1) approximately prior to a time point (Tmax2) at which the viral-replication modulator reaches a maximum biological fluid concentration (Cmax2). Time of administration of the therapeutic composition is illustrated as T0, with T1 and T2 illustrating the time points at which each agent, or component, is no longer detectable in the biological fluid. For example, T1 illustrates the time point at which the virus entry inhibitor is no longer detectable in the biological fluid, while T2 illustrates the time point at which the viral-replication modulator is no longer detectable in the biological fluid.

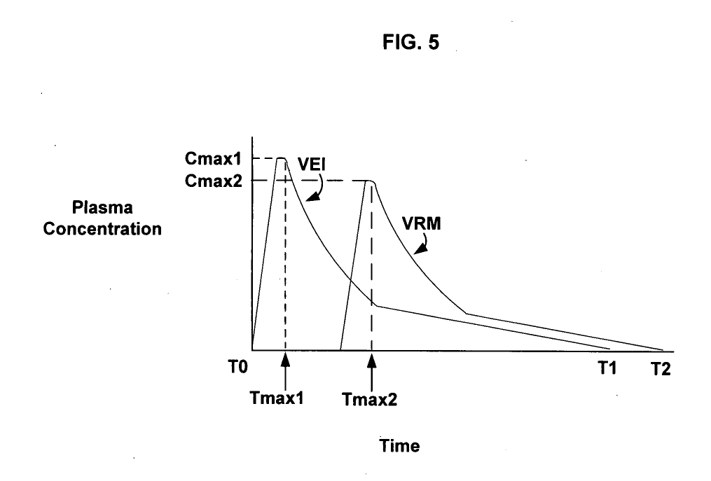

As illustrated in FIG. 5, in one example biological fluid concentration profile, the therapeutic composition includes at least one virus entry inhibitor (VEI) and at least one viral-replication modulator (VRM), wherein the at least one virus entry inhibitor is formulated to reach a maximum biological fluid concentration (Cmax1) by at least one time point (Tmax1) approximately prior to a time point (Tmax2) at which the viral-replication modulator reaches a maximum biological fluid concentration (Cmax2). Time of administration of the therapeutic composition is illustrated as T0, with T1 and T2 illustrating the time point at which the at least one virus entry inhibitor and the at least one viral-replication modulator are no longer detectable in the biological fluid, respectively.

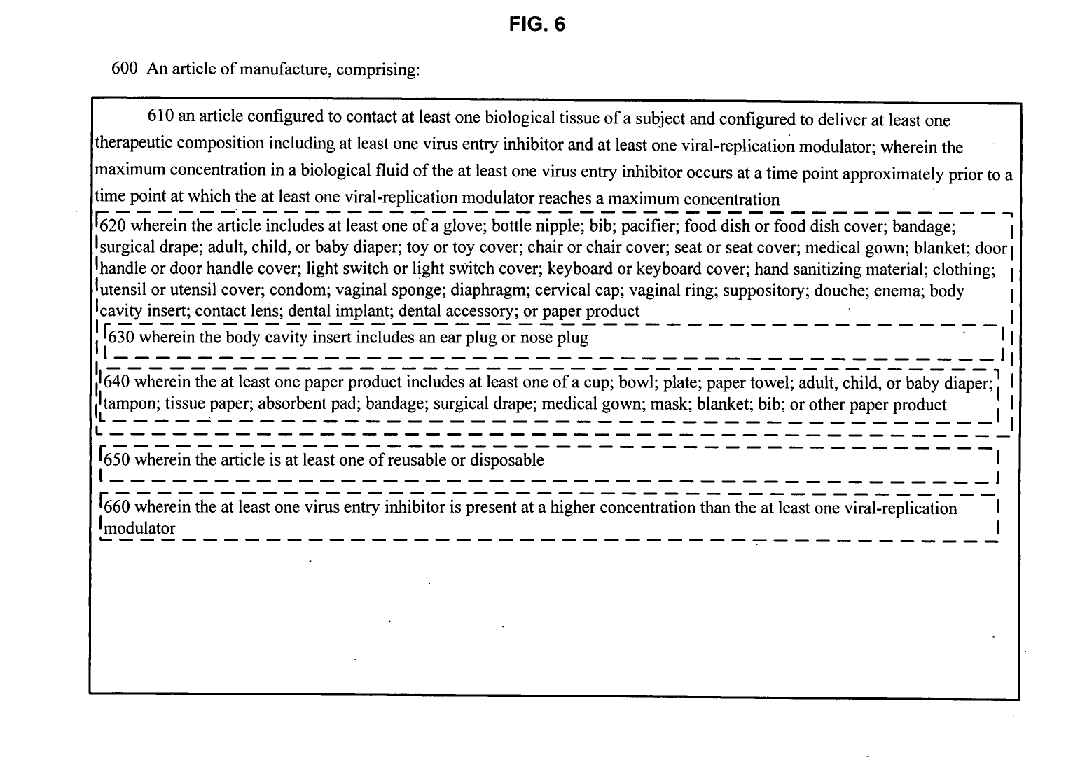

As illustrated in FIG. 6, an article of manufacture 600, comprises an article 610 formulated to contact at least one biological tissue of a subject. In an embodiment 620, the article includes at least one therapeutic composition of at least one virus entry inhibitor and at least one viral-replication modulator. In an embodiment 625, the at least one virus entry inhibitor is formulated to reach a maximum biological fluid concentration approximately prior to a time point at which the at least one viral-replication modulator reaches a maximum biological fluid concentration. In an embodiment 630, the article includes at least one of a glove; bottle nipple; bib; pacifier; food dish or food dish cover; bandage; surgical drape; adult, child, or baby diaper; toy or toy cover; chair or chair cover; seat or seat cover; medical gown; blanket; door handle or door handle cover; light switch or light switch cover; keyboard or keyboard cover; hand sanitizing material; clothing; utensil or utensil cover; condom; vaginal sponge; diaphragm; cervical cap; vaginal ring; suppository; douche; enema; body cavity insert; contact lens; dental implant; dental accessory; or paper product. In an embodiment 640, the body cavity insert includes an ear plug or a nose plug. In an embodiment 650, the at least one paper product includes at least one of a cup; bowl; plate; paper towel; adult, child, or baby diaper; tampon; tissue paper; absorbent pad; bandage; surgical drape; medical gown; mask; blanket; bib; or other paper product. In an embodiment 660, the article is at least one of reusable or disposable. In an embodiment 670, the at least one virus entry inhibitor is present at a higher concentration than the at least one viral-replication modulator.

Prior to determining a treatment regimen, additional information regarding the physiological status of the subject or tissue may be gathered and assessed. For example, information may be collected on a subject's medical history or familial history, including genetic or proteomic information. The subject's information may include organ function, disease state, genetic or proteomic variability that may influence absorption, metabolic or excretion pathways, circadian rhythm, age, gender, weight, or other factors. The individualized medical evaluation can include a genetic profile of the subject regarding genes, genetic mutations or genetic polymorphisms that indicate risk factors that affect disease related to viral infection. Other factors that may be considered in determining a delivery regimen include, but are not limited to route of administration, drug interactions, herbal or vitamin supplements, food or beverages consumed by the subject, and the subject's compliance with the regimen.

A genetic polymorphism or genetic mutation in a genetic profile of a subject that encodes a component of one or more cell receptors, cell ligands, or cell signaling molecules may affect the levels of virus allowed to enter or proliferate in a subject or biological tissue(s). Thus, genetic profiling may be used prior to, or during the initiation of a treatment regimen including providing one or more agents that modulate one or more cell receptors, cell ligands, or cell signaling molecules, in order to assess whether the subject has any genetic mutations or genetic polymorphisms that may be correlated with a particular viral infection risk or immune response.

A genetic polymorphism or mutation may indicate how a particular subject will respond to a specific delivery regimen. For example, genomic DNA used in genetic profiling may be isolated from any biological sample which contains the DNA of that subject, including but not limited to blood, saliva, cheek swab, epithelium, urine, or other tissue or bodily fluid. For example, genomic DNA may be extracted from whole blood or from isolated peripheral blood leukocytes isolated by differential centrifugation from whole blood using a commercial kit (See e.g., QIAmp DNA Blood Mini Kit, Qiagen, Valencia, Calif.) according to the manufacturer's instructions.

Medical evaluation of the subject or tissue for genetic or proteomic profiling or genetic or proteomic testing may be provided as a current determination of genetic risk factors in the subject or tissue, or as part of the subject's medical history. Genetic profiling or genetic testing may be determined by using a variety of methods including but not limited to restriction landmark genomic scanning (RLGS), Southern blot analysis combined with restriction fragment length polymorphism (RFLP), fluorescence in situ hybridization (FISH), enzyme mismatch cleavage (EMC) of nucleic acid heteroduplexes, ligase chain reaction (LCR) or polymerase chain reaction (PCR) based methods. Analysis of one or more single nucleotide polymorphisms (SNPs) may also be used for genetic profiling.

Restriction fragment landmark genomic scanning (RLGS) may be used to scan an entire mammalian genome. As such, genomic DNA is digested with restriction enzymes to generate large DNA fragments. The fragments are separated on an agarose gel, digested with one or more restriction enzymes within the agarose gel, and then separated in a second dimension by polyacrylamide gel electrophoresis (PAGE) (See e.g., Tawata, et al., Comb., Chem. High Throughput Screen, Vol. 3, pp., 1-9 (2000), which is herein incorporated by reference). The DNA may be labeled prior to digestion, or the fragments may be stained nonspecifically as with an intercalating dye, for example. The resulting pattern may be compared with pre-established norms to detect genetic mutations.

Restriction fragment length polymorphism (RFLP) is similar to restriction fragment landmark genomic scanning in that the genomic DNA is digested with specific restriction enzymes and separated on an agarose gel. The separated DNA is transferred to a membrane and the fragments are visualized using hybridization analysis and gene specific probes.

A variety of PCR related methods may be used for genetic profiling and may be used to detect both known and unknown mutations and polymorphisms (See e.g., Tawata, et al., Comb. Chem. High Throughput Screen., Vol. 3, pp. 1-9 (2000), which is herein incorporated by reference). For known mutations and polymorphisms, specific PCR oligonucleotide probes are designed to bind directly to the mutation or polymorphism or proximal to the mutation or polymorphism. For example, PCR may be used in combination with RFLP. In this instance, a DNA fragment or fragments generated by PCR with primers on either side of the mutation or polymorphism site are treated with restriction enzymes and separated by agarose gel electrophoresis. The fragments themselves may be detected using an intercalating dye such as, for example, ethidium bromide. An aberrant banding pattern may be observed if mutations exist within the restriction sites. PAGE may be used to detect single base differences in the size of a fragment.

Alternatively, PCR may be used in combination with DNA sequencing for genetic profiling. For example, PCR primers may be designed that bind to either side of a potential mutation site on the target DNA and generate a PCR fragment that spans a potential mutation site. The PCR fragment is either directly sequenced or subcloned into a cloning vector and subsequently sequenced using standard molecular biology techniques.

Alternatively, a mutation or polymorphism may be screened using comparative genomic hybridization (CGH) (See e.g., Pinkel & Albertson, Nat. Gen. Vol. 37:S11-S17 (2005), which is herein incorporated by reference). In this instance, “normal” genomic DNA and test genomic DNA are differentially labeled and hybridized to metaphase chromosomes or DNA microarrays. The relative hybridization signal at a given location is proportional to the relative copy number of the sequences in the reference and test genomes. Arrays may be generated using DNA obtained from, for example, bacterial artificial chromosomes (BACs) or PCR.

Analysis of one or more single nucleotide polymorphism (SNP) may be used for genetic profiling. A SNP is a DNA sequence variation in which a single nucleotide in the genomic sequence differs between members of a species (or between paired chromosomes of an individual). For a variation to be considered a SNP it must occur in at least 1% of the population. Most SNPs do not affect protein function, and/or are not responsible for a disease state, but they may serve as biological markers for pinpointing an altered protein or disease on the human genome map as they are often located near a gene found to be associated with a certain disease. Occasionally, a SNP may actually affect protein function and/or cause a disease and, therefore, can be used to search for and isolate a specific gene, e.g., a T to C mutation in the CYP17 gene which affects enzyme function. The pattern of SNPs in a subject's genomic DNA may be compared with information in databases in an association study to determine effect on protein function and/or risk of disease development. SNPs may be identified using PCR and DNA sequencing as described above. Alternatively, SNP genotyping may be done using high throughput array analysis (See e.g., Applied BioSystems, ABI PRISM, 3100 Genetic Analyzer with 22-cm Capillary Array; Syvanen, et al., Nat. Genet., Vol. 37, pp. S5-S10 (2005) which is herein incorporated by reference). A growing number of web-based databases are available for finding information regarding SNPs and protein function and/o disease associations (See e.g., International HapMap Project on the worldwide web at //snp.cshl.org; Nature 449: 851-861, 2007; National Center Biotechnology Information (NCBI) Single Nucleotide Polymorphisms, on the worldwide web at ncbi.nlm.nih.gov/projects/SNP/, which is herein incorporated by reference).

Studies have reported that homozygosity for a 32 base pair deletion in the CCR5 allele of the chemokine receptor (CCR5) provides resistance against HIV-1 infection. See, for example, Huffer, et al., Abstract, New Eng. J. Med., vol. 360, no. 7, pp. 692-698 (2009), which is incorporated herein by reference. This mutation, CCR5 delta32/delta32, is found largely in European populations, and is believed to be a mutation that arose relatively recently in evolutionary. Id.

The disclosure further provides kits including at least one therapeutic composition, device, article of manufacture, or method disclosed herein. Any particular kit may also contain instructional material teaching the methodologies and uses of the therapeutic composition or method, as described herein.

For example, in an embodiment, the kit includes a single dose or multi-dose package of the therapeutic composition (e.g., oral, topical, transdermal formulations). In an embodiment, the therapeutic composition includes FDA-approved agents. Instructions accompanying the dosage would indicate at least one of the appropriate dosage, method of dosing (e.g., ingesting, applying, etc.), time period for dosing prior to, during, or subsequent to potential exposure to at least one virus, contraindications, any required or suggested modifications associated with food intake or other substances, or any other behaviors that can reduce exposure or infection with at least one virus.

Additionally, the therapeutic compositions described herein can be lyophilized to dry form for ease in transportation and storage (e.g., as part of a kit). The therapeutic compositions described herein can be stored, for example, in a sealed vial, ampule, or similar packaging. In the case where the therapeutic composition has been lyophilized, it is dissolved or suspended (e.g. in sterilized distilled water, saline, phosphate buffered saline, Tris buffer, sodium phosphate, or other solvent) prior to administration.

In an embodiment, an article of manufacture includes the at least one anti-viral therapeutic composition. For example, in an embodiment, the article of manufacture includes at least one of a glove, bottle nipple, bib, pacifier, toy or toy cover, chair or chair cover, seat or seat cover, door handle or door handle cover, light switch or light switch cover, keyboard or keyboard cover, computer mouse, hand sanitizing material, clothing, utensil or utensil cover, condom, sponge, diaphragm, cervical cap, vaginal ring, suppository, douche, enema, body cavity insert, contact lens, dental implant, dental accessory, or paper product. In an embodiment, the body cavity insert includes an ear plug or a nose plug. In an embodiment, the paper product includes at least one paper-based or plant-based product formulated for contacting at least one biological tissue of a subject. In an embodiment, the at least one paper product includes at least one of a cup; bowl; plate; paper towel; adult, child, or baby diaper; tampon; tissue paper; absorbent pad; bandage; surgical drape; medical gown; mask; blanket; bib; or other paper product. In an embodiment, the article is at least one of reusable or disposable. In an embodiment, an article of manufacture described herein is included in a kit disclosed herein.

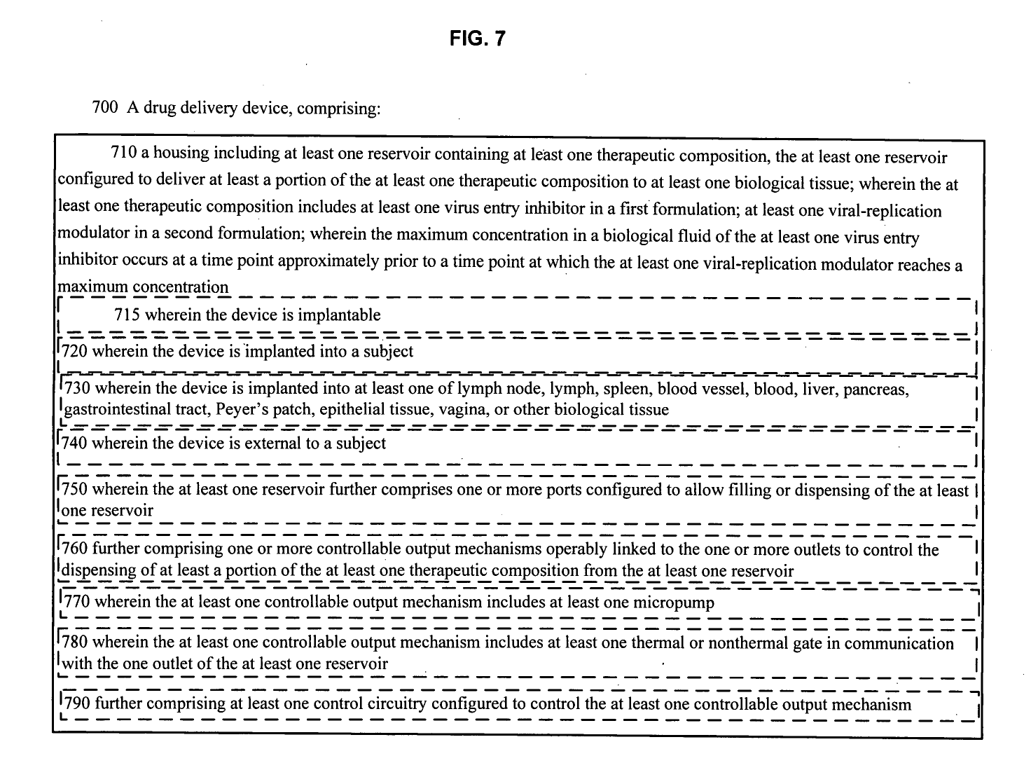

As described in FIG. 7, in an embodiment, a drug delivery device 700, comprises 710 a housing including at least one reservoir containing at least one therapeutic composition, the at least one reservoir configured to deliver at least a portion of the at least one therapeutic composition to at least one biological tissue, wherein the at least one therapeutic composition includes at least one virus entry inhibitor in a first formulation; at least one viral-replication modulator in a second formulation; wherein the maximum concentration in a biological fluid of the at least one virus entry inhibitor occurs at a time point approximately prior to a time point at which the at least one viral-replication modulator reaches a maximum concentration. In an embodiment 715, the device is implantable. In an embodiment 720, the device is implanted into a subject.

In an embodiment 730, the drug delivery device is implanted into at least one of lymph node, lymph, spleen, blood vessel, blood, liver, pancreas, gastrointestinal tract, Peyer's patch, epithelial tissue, vagina, or other biological tissue. In an embodiment 740, the device is external to a subject. In an embodiment 750, the at least one reservoir further comprises one or more ports configured to allow filling or dispensing of the at least one reservoir. In an embodiment 760, the device further comprises one or more controllable output mechanisms operably linked to the one or more outlets to control the dispensing of at least a portion of the at least one therapeutic composition from the at least one reservoir. In an embodiment 770, the at least one controllable output mechanism includes at least one micropump. In an embodiment 780, the at least one controllable output mechanism includes at least one thermal or nonthermal gate in communication with the one outlet of the at least one reservoir. In an embodiment 790, the device further comprises at least one control circuitry configured to control the at least one controllable output mechanism.

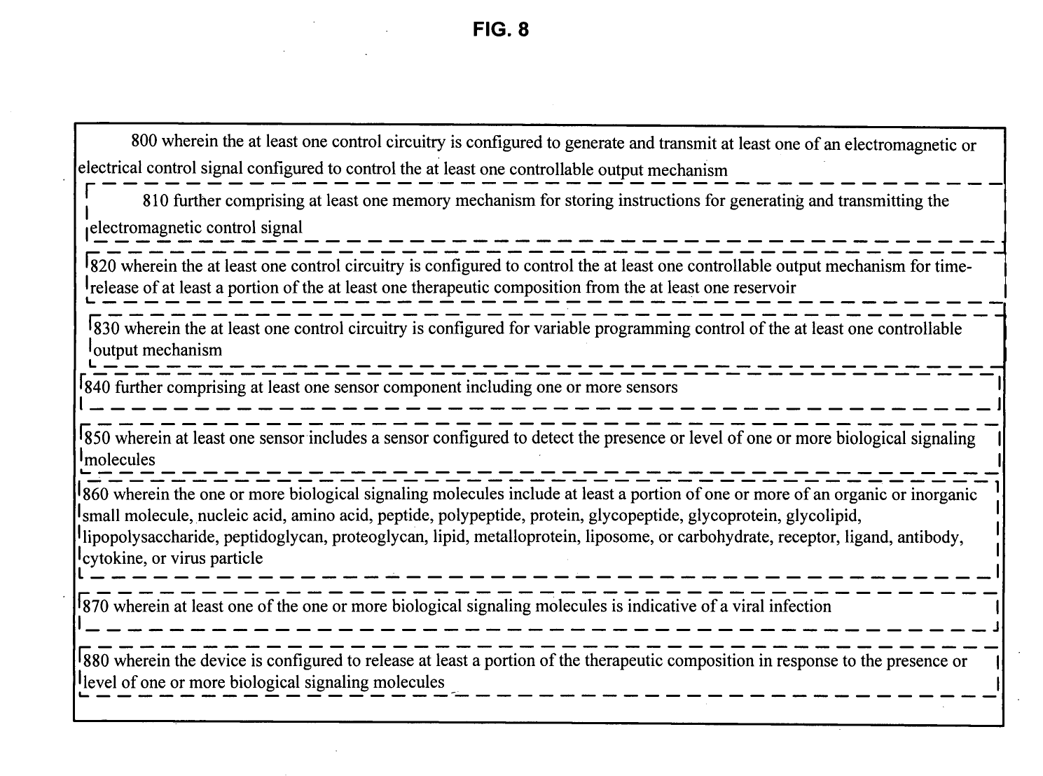

As indicated in FIG. 8, in an embodiment 800, the at least one control circuitry is configured to generate and transmit at least one of an electromagnetic or electrical control signal configured to control the at least one controllable output mechanism. In an embodiment 810, the device further comprises at least one memory mechanism for storing instructions for generating and transmitting the electromagnetic control signal. In an embodiment 820, the at least one control circuitry is configured to control the at least one controllable output mechanism for time-release of at least a portion of the at least one therapeutic composition from the at least one reservoir.

In an embodiment 830, the at least one control circuitry is configured for variable programming control of the at least one controllable output mechanism. In an embodiment 840, the drug delivery device further comprises at least one sensor component including one or more sensors. In an embodiment 850, at least one sensor includes a sensor configured to detect the presence or level of one or more biological signaling molecules. In an embodiment 860, the one or more biological signaling molecules include at least a portion of one or more of an organic or inorganic small molecule, nucleic acid, amino acid, peptide, polypeptide, protein, glycopeptide, glycoprotein, glycolipid, lipopolysaccharide, peptidoglycan, proteoglycan, lipid, metalloprotein, liposome, or carbohydrate, receptor, ligand, antibody, cytokine, or virus particle. In an embodiment 870, at least one of the one or more biological signaling molecules is indicative of a viral infection. In an embodiment 880, the device is configured to release at least a portion of the therapeutic composition in response to the presence or level of one or more biological signaling molecules.

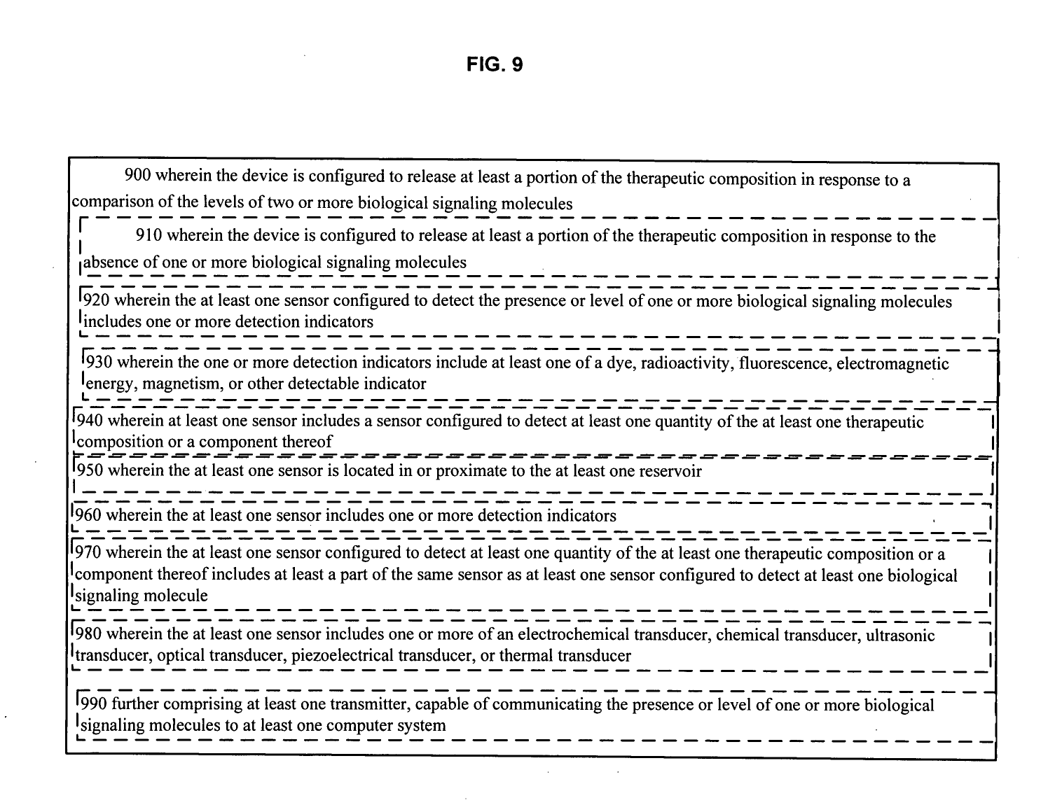

As indicated in FIG. 9, in an embodiment 900, the device is configured to release at least a portion of the therapeutic composition in response to a comparison of the levels of two or more biological signaling molecules. In an embodiment 910, the device is configured to release at least a portion of the therapeutic composition in response to the absence of one or more biological signaling molecules. In an embodiment 920, the at least one sensor configured to detect the presence or level of one or more biological signaling molecules includes one or more detection indicators. In an embodiment 930, the one or more detection indicators include at least one of a dye, radioactivity, fluorescence, electromagnetic energy, magnetism, or other detectable indicator. In an embodiment 940, at least one sensor includes a sensor configured to detect at least one quantity of the at least one therapeutic composition or a component thereof. In an embodiment 950, the at least one sensor is located in or proximate to the at least one reservoir. In an embodiment 960, the at least one sensor includes one or more detection indicators. In an embodiment 970, the at least one sensor configured to detect at least one quantity of the at least one therapeutic composition or a component thereof includes at least a part of the same sensor as at least one sensor configured to detect at least one biological signaling molecule.

In an embodiment 980, the at least one sensor includes one or more of an electrochemical transducer, chemical transducer, ultrasonic transducer, optical transducer, piezoelectrical transducer, or thermal transducer. In an embodiment 990, the device further comprises at least one transmitter, capable of communicating the presence or level of one or more biological signaling molecules to at least one computer system.

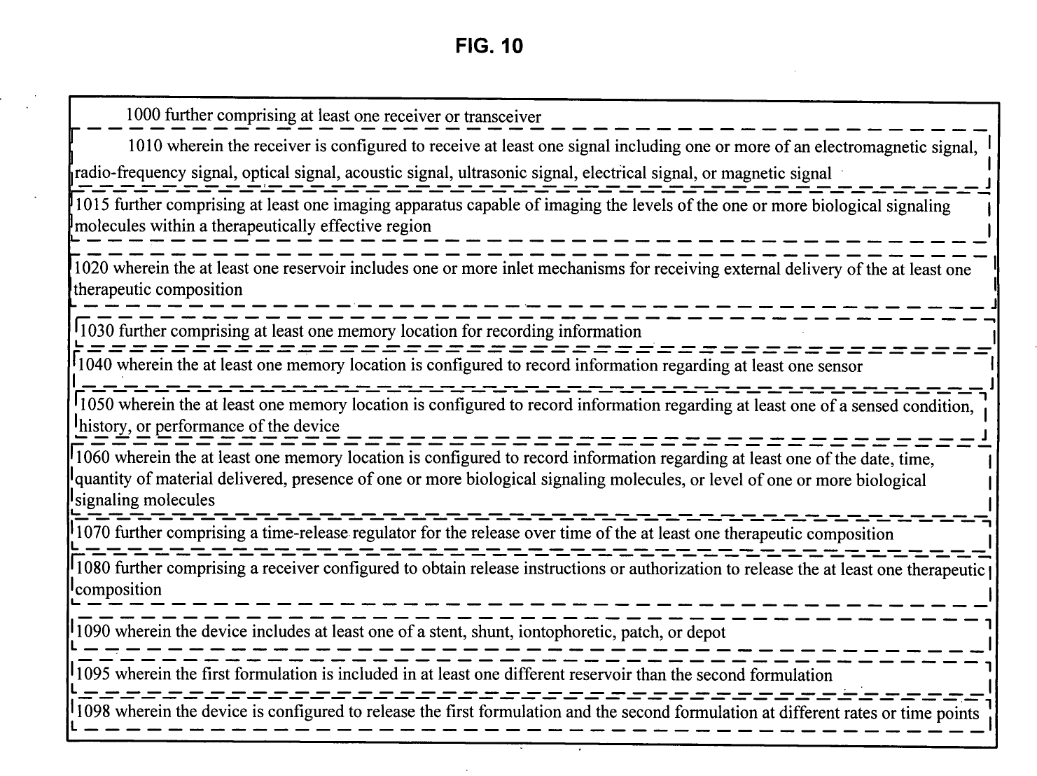

As indicated in FIG. 10, in an embodiment 1000, the drug delivery device further comprises at least one receiver or transceiver. In an embodiment 1010, the receiver is configured to receive at least one signal including one or more of an electromagnetic signal, radio-frequency signal, optical signal, acoustic signal, ultrasonic signal, electrical signal, or magnetic signal. In an embodiment 1015, the drug delivery device further comprises at least one imaging apparatus capable of imaging the levels of the one or more biological signaling molecules within a therapeutically effective region. In an embodiment 1020, the at least one reservoir includes one or more inlet mechanisms for receiving external delivery of the at least one therapeutic composition. In an embodiment 1030, the drug delivery device further comprises at least one memory location for recording information. In an embodiment 1040, the at least one memory location is configured to record information regarding at least one sensor. In an embodiment 1050, the at least one memory location is configured to record information regarding at least one of a sensed condition, history, or performance of the device.

In an embodiment 1060, the at least one memory location is configured to record information regarding at least one of the date, time, quantity of material delivered, presence of one or more biological signaling molecules, or level of one or more biological signaling molecules. In an embodiment 1070, the drug delivery device further comprises a time-release regulator for the release over time of the at least one therapeutic composition. In an embodiment 1080, the drug delivery device further comprises a receiver configured to obtain release instructions or authorization to release the at least one therapeutic composition. In an embodiment 1090, the device includes at least one of a stent, shunt, iontophoretic, patch, or depot. In an embodiment 1095, the first formulation is included in at least one different reservoir than the second formulation. In an embodiment 1098, the device is configured to release the first formulation and the second formulation at different rates or time points.

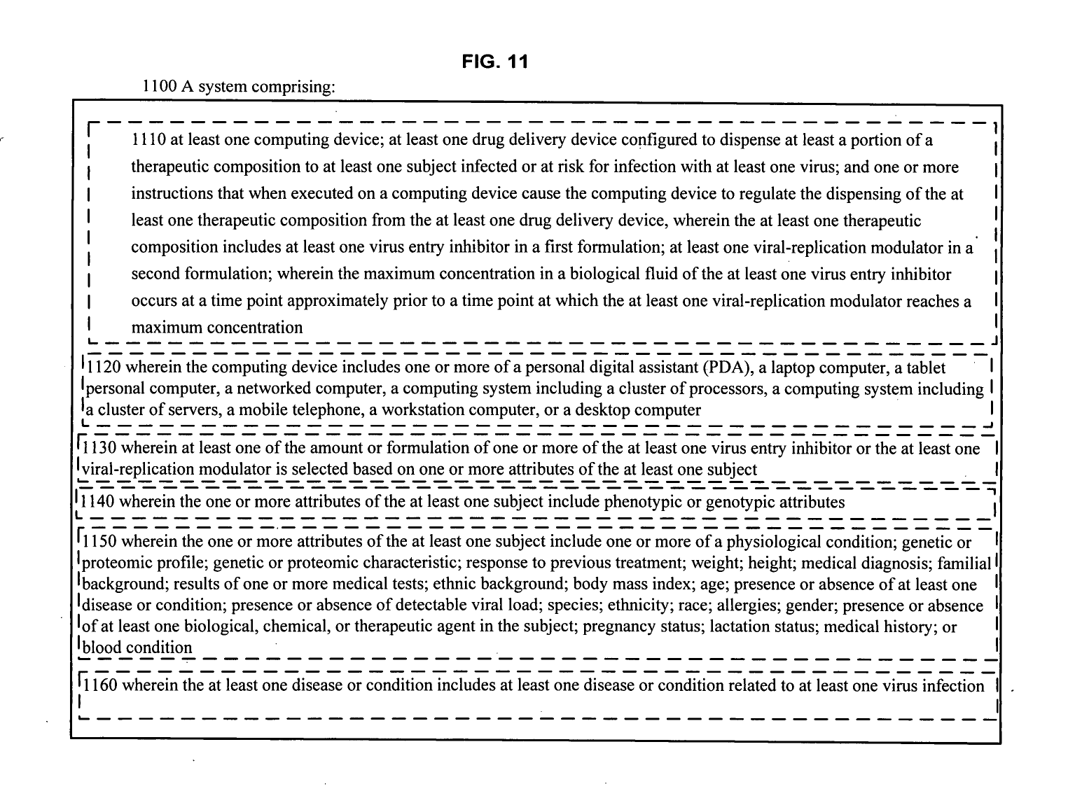

As indicated in FIG. 11, a system 1100, includes at least one computing device; at least one drug delivery device configured to dispense at least a portion of a therapeutic composition to at least one subject infected or at risk for infection with at least one virus; and one or more instructions that when executed on a computing device cause the computing device to regulate the dispensing of the at least one therapeutic composition from the at least one drug delivery device, wherein the at least one therapeutic composition includes at least one virus entry inhibitor in a first formulation; at least one viral-replication modulator in a second formulation; wherein the maximum concentration in a biological fluid of the at least one virus entry inhibitor occurs at a time point approximately prior to a time point at which the at least one viral-replication modulator reaches a maximum concentration.

In an embodiment 1120, wherein the computing device includes one or more of a personal digital assistant (PDA), a laptop computer, a tablet personal computer, a networked computer, a computing system including a cluster of processors, a computing system including a cluster of servers, a mobile telephone, a workstation computer, or a desktop computer. In an embodiment 1130, at least one of the amount or formulation of one or more of the at least one virus entry inhibitor or the at least one viral-replication modulator is selected based on one or more attributes of the at least one subject. In an embodiment 1140, the one or more attributes of the at least one subject include phenotypic or genotypic attributes. In an embodiment 1150, the one or more attributes of the at least one subject include one or more of a physiological condition; genetic or proteomic profile; genetic or proteomic characteristic; response to previous treatment; weight; height; medical diagnosis; familial background; results of one or more medical tests; ethnic background; body mass index; age; presence or absence of at least one disease or condition; presence or absence of detectable viral load; species; ethnicity; race; allergies; gender; presence or absence of at least one biological, chemical, or therapeutic agent in the subject; pregnancy status; lactation status; medical history; or blood condition. In an embodiment 1160, the at least one disease or condition includes at least one disease or condition related to at least one virus infection.

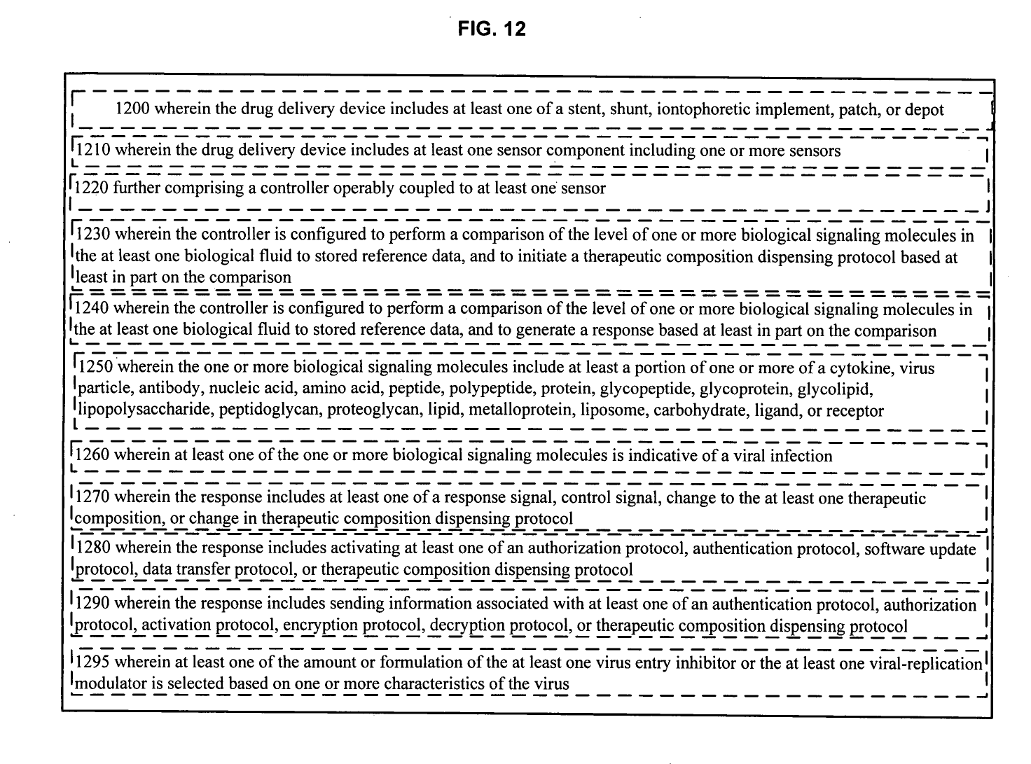

As indicated in FIG. 12, in an embodiment 1200, the drug delivery device includes at least one of a stent, shunt, iontophoretic implement, patch, or depot. In an embodiment 1210, the drug delivery device includes at least one sensor component including one or more sensors. In an embodiment 1220, the system further comprises a controller operably coupled to at least one sensor. In an embodiment 1230, the controller is configured to perform a comparison of the level of one or more biological signaling molecules in the at least one biological fluid to stored reference data, and to initiate a therapeutic composition dispensing protocol based at least in part on the comparison. In an embodiment 1240, the controller is configured to perform a comparison of the level of one or more biological signaling molecules in the at least one biological fluid to stored reference data, and to generate a response based at least in part on the comparison. In an embodiment 1250, the one or more biological signaling molecules include at least a portion of one or more of a cytokine, virus particle, antibody, nucleic acid, amino acid, peptide, polypeptide, protein, glycopeptide, glycoprotein, glycolipid, lipopolysaccharide, peptidoglycan, proteoglycan, lipid, metalloprotein, liposome, carbohydrate, ligand, or receptor. In an embodiment 1260, at least one of the one or more biological signaling molecules is indicative of a viral infection. In an embodiment 1270, the response includes at least one of a response signal, control signal, change to the at least one therapeutic composition, or change in therapeutic composition dispensing protocol. In an embodiment 1280, the response includes activating at least one of an authorization protocol, authentication protocol, software update protocol, data transfer protocol, or therapeutic composition dispensing protocol. In an embodiment 1290, the response includes sending information associated with at least one of an authentication protocol, authorization protocol, activation protocol, encryption protocol, decryption protocol, or therapeutic composition dispensing protocol. In an embodiment 1295, at least one of the amount or formulation of the at least one virus entry inhibitor or the at least one viral-replication modulator is selected based on one or more characteristics of the virus.

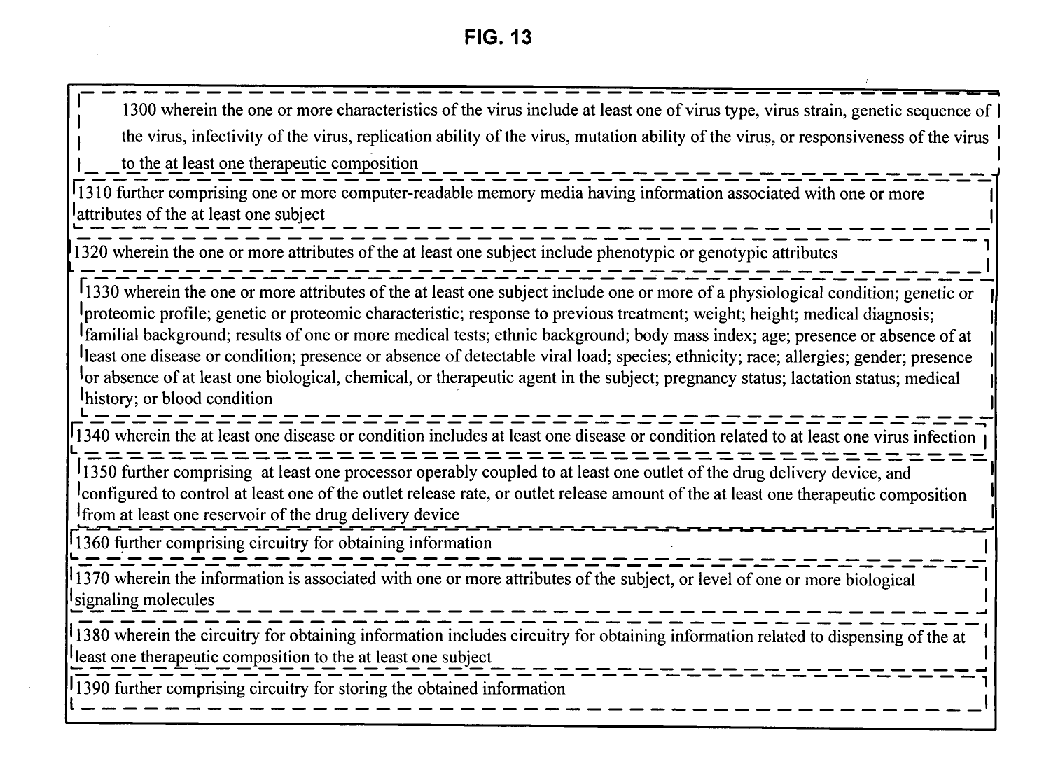

As indicated in FIG. 13, in an embodiment 1300, the one or more characteristics of the virus include at least one of virus type, virus strain, genetic sequence of the virus, infectivity of the virus, replication ability of the virus, mutation ability of the virus, or responsiveness of the virus to the at least one therapeutic composition. In an embodiment 1310, the system further comprises one or more computer-readable memory media having information associated with one or more attributes of the at least one subject. In an embodiment 1320, the one or more attributes of the at least one subject include phenotypic or genotypic attributes. In an embodiment 1330, one or more attributes of the at least one subject include one or more of a physiological condition; genetic or proteomic profile; genetic or proteomic characteristic; response to previous treatment; weight; height; medical diagnosis; familial background; results of one or more medical tests; ethnic background; body mass index; age; presence or absence of at least one disease or condition; presence or absence of detectable viral load; species; ethnicity; race; allergies; gender; presence or absence of at least one biological, chemical, or therapeutic agent in the subject; pregnancy status; lactation status; medical history; or blood condition. In an embodiment 1340, the at least one disease or condition includes at least one disease or condition related to at least one virus infection. In an embodiment 1350, the system further comprises at least one processor operably coupled to at least one outlet of the drug delivery device, and configured to control at least one of the outlet release rate, or outlet release amount of the at least one therapeutic composition from at least one reservoir of the drug delivery device. In an embodiment 1360, the system further comprises circuitry for obtaining information. In an embodiment 1370, the information is associated with one or more attributes of the subject, or level of one or more biological signaling molecules. In an embodiment 1380, the circuitry for obtaining information includes circuitry for obtaining information related to dispensing of the at least one therapeutic composition to the at least one subject. In an embodiment 1390, the system further comprises circuitry for storing the obtained information.

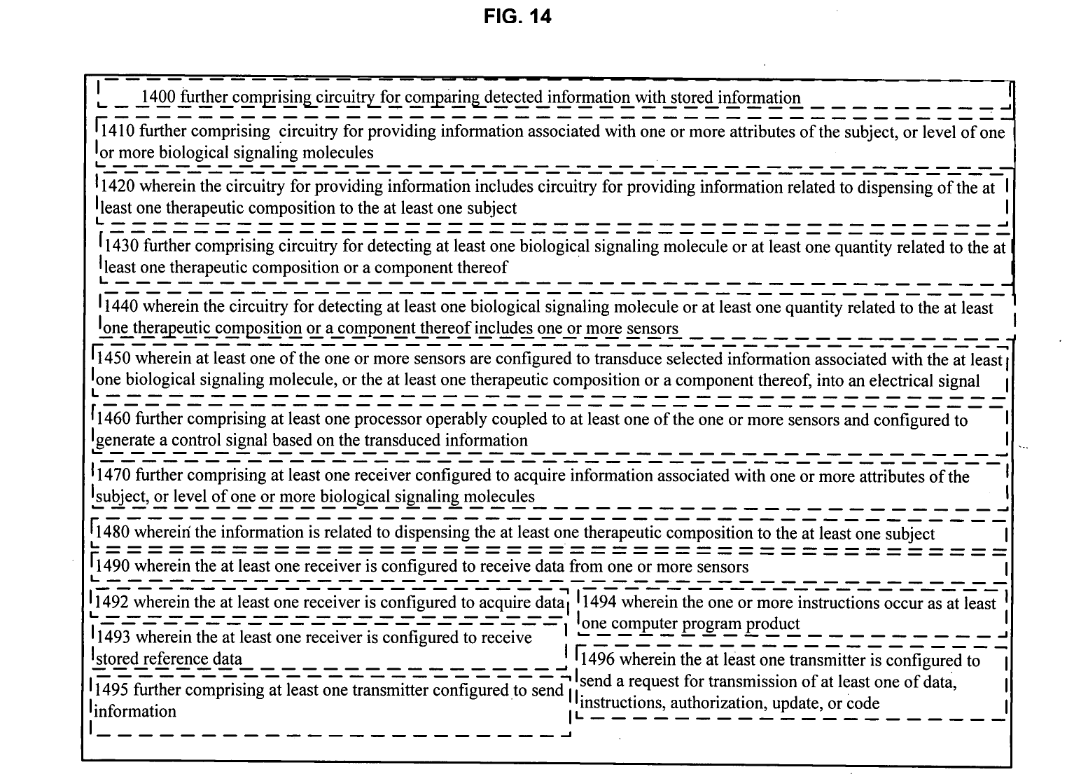

As indicated in FIG. 14, in an embodiment 1400, the system further comprises circuitry for comparing detected information with stored information. In an embodiment 1410, the system further comprises circuitry for providing information associated with one or more attributes of the subject, or level of one or more biological signaling molecules. In an embodiment 1420, the circuitry for providing information includes circuitry for providing information related to dispensing of the at least one therapeutic composition to the at least one subject. In an embodiment 1430, the system further comprises circuitry for detecting at least one biological signaling molecule or at least one quantity related to the at least one therapeutic composition or a component thereof. In an embodiment 1440, the circuitry for detecting at least one biological signaling molecule or at least one quantity related to the at least one therapeutic composition or a component thereof includes one or more sensors. In an embodiment 1450, at least one of the one or more sensors are configured to transduce selected information associated with the at least one biological signaling molecule, or the at least one therapeutic composition or a component thereof, into an electrical signal.

In an embodiment 1460, the system further comprises at least one processor operably coupled to at least one of the one or more sensors and configured to generate a control signal based on the transduced information. In an embodiment 1470, the system further comprises at least one receiver configured to acquire information associated with one or more attributes of the subject, or level of one or more biological signaling molecules. In an embodiment 1480, the information is related to dispensing the at least one therapeutic composition to the at least one subject. In an embodiment 1490, the at least one receiver is configured to receive data from one or more sensors. In an embodiment 1492, the at least one receiver is configured to acquire data. In an embodiment 1493, the at least one receiver is configured to receive stored reference data. In an embodiment 1494, the one or more instructions occur as at least one computer program product. In an embodiment 1495, the system further comprises at least one transmitter configured to send information. In an embodiment 1496, the at least one transmitter is configured to send a request for transmission of at least one of data, instructions, authorization, update, or code.

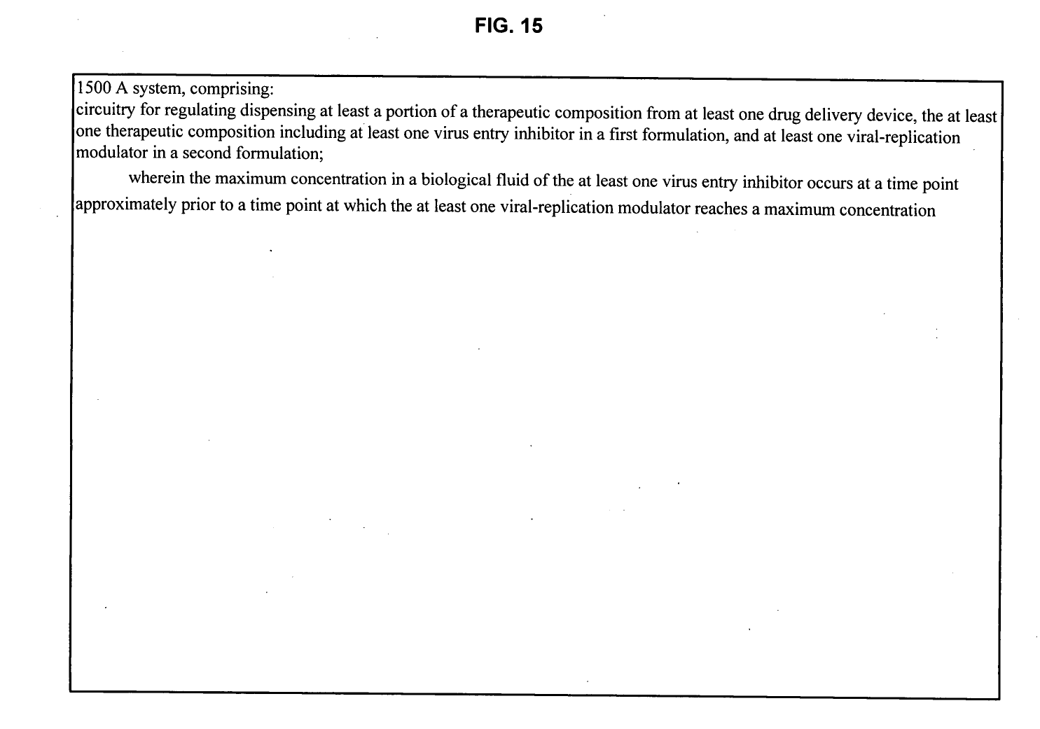

As indicated in FIG. 15, in an embodiment 1500, a system, comprises circuitry for regulating dispensing at least a portion of a therapeutic composition from at least one drug delivery device, the at least one therapeutic composition including at least one virus entry inhibitor in a first formulation, and at least one viral-replication modulator in a second formulation; wherein the maximum concentration in a biological fluid of the at least one virus entry inhibitor occurs at a time point approximately prior to a time point at which the at least one viral-replication modulator reaches a maximum concentration.

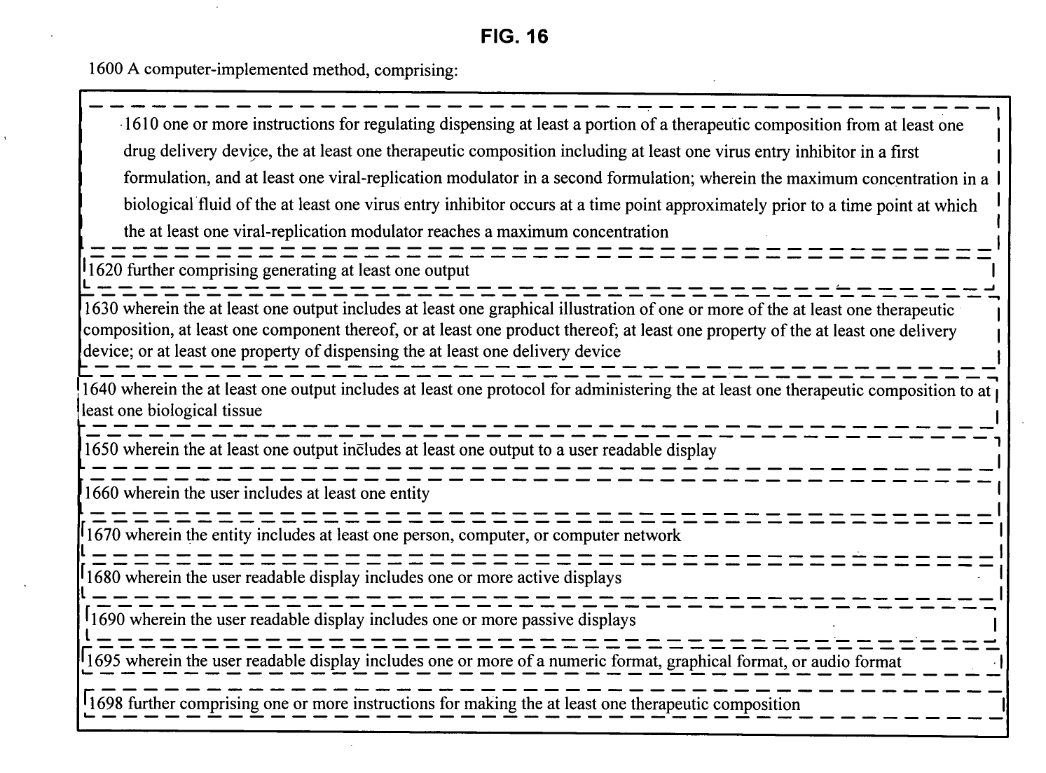

As indicated in FIG. 16, a computer-implemented method 1600, comprises one or more instructions for regulating dispensing at least a portion of a therapeutic composition from at least one drug delivery device, the at least one therapeutic composition including at least one virus entry inhibitor in a first formulation, and at least one viral-replication modulator in a second formulation; wherein the maximum concentration in a biological fluid of the at least one virus entry inhibitor occurs at a time point approximately prior to a time point at which the at least one viral-replication modulator reaches a maximum concentration. In an embodiment 1620, the computer-implemented method further comprises generating at least one output. In an embodiment 1630, the at least one output includes at least one graphical illustration of one or more of the at least one therapeutic composition, at least one component thereof, or at least one product thereof; at least one property of the at least one delivery device; or at least one property of dispensing the at least one delivery device. In an embodiment 1640, the at least one output includes at least one protocol for administering the at least one therapeutic composition to at least one biological tissue. In an embodiment 1650, the at least one output includes at least one output to a user readable display. In an embodiment 1660, the user includes at least one entity. In an embodiment 1670, the entity includes at least one person, computer, or computer network. In an embodiment 1680, the user readable display includes one or more active displays. In an embodiment 1690, the user readable display includes one or more passive displays. In an embodiment 1695, the user readable display includes one or more of a numeric format, graphical format, or audio format. In an embodiment 1698, the computer-implemented method further comprises one or more instructions for making the at least one therapeutic composition.

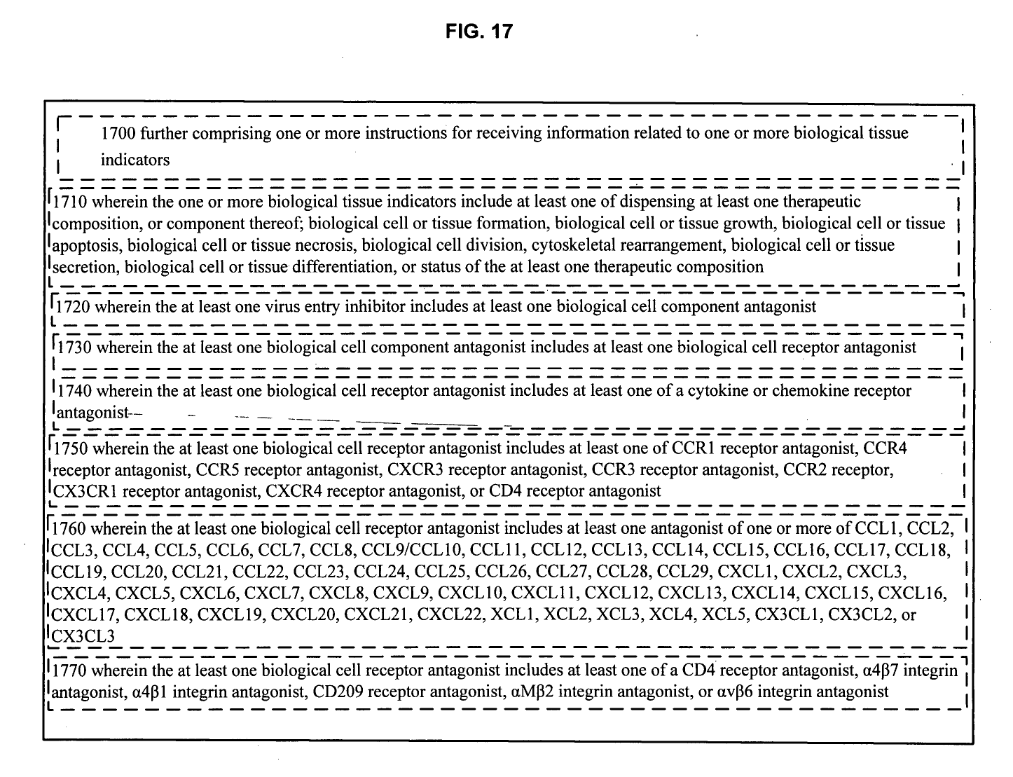

As indicated in FIG. 17, in an embodiment 1700, the computer-implemented method further comprises one or more instructions for receiving information related to one or more biological tissue indicators. In an embodiment 1710, the one or more biological tissue indicators include at least one of dispensing at least one therapeutic composition, or component thereof; biological cell or tissue formation, biological cell or tissue growth, biological cell or tissue apoptosis, biological cell or tissue necrosis, biological cell division, cytoskeletal rearrangement, biological cell or tissue secretion, biological cell or tissue differentiation, or status of the at least one therapeutic composition. In an embodiment 1720, the at least one virus entry inhibitor includes at least one biological cell component antagonist. In an embodiment 1730, the at least one biological cell component antagonist includes at least one biological cell receptor antagonist. In an embodiment 1740, the at least one biological cell receptor antagonist includes at least one of a cytokine or chemokine receptor antagonist. In an embodiment 1750, the at least one biological cell receptor antagonist includes at least one of CCR1 receptor antagonist, CCR4 receptor antagonist, CCR5 receptor antagonist, CXCR3 receptor antagonist, CCR3 receptor antagonist, CCR2 receptor, CX3CR1 receptor antagonist, CXCR4 receptor antagonist, or CD4 receptor antagonist. In an embodiment 1760, the at least one biological cell receptor antagonist includes at least one antagonist of one or more of CCL1, CCL2, CCL3, CCL4, CCL5, CCL6, CCL7, CCL8, CCL9/CCL10, CCL11, CCL12, CCL13, CCL14, CCL15, CCL16, CCL17, CCL18, CCL19, CCL20, CCL21, CCL22, CCL23, CCL24, CCL25, CCL26, CCL27, CCL28, CCL29, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCL17, CXCL18, CXCL19, CXCL20, CXCL21, CXCL22, XCL1, XCL2, XCL3, XCL4, XCL5, CX3CL1, CX3CL2, or CX3CL3. In an embodiment 1770, the at least one biological cell receptor antagonist includes at least one of a CD4 receptor antagonist, α4β7 integrin antagonist, α4β1 integrin antagonist, CD209 receptor antagonist, αMβ2 integrin antagonist, or αvβ6 integrin antagonist.

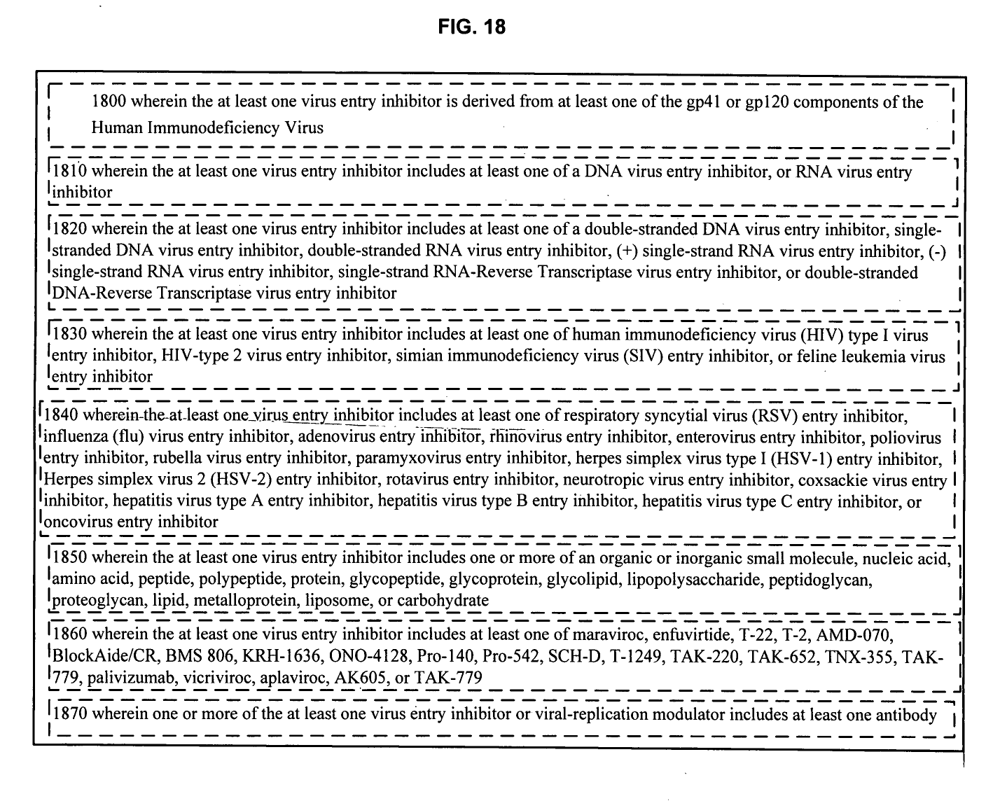

As indicated in FIG. 18, in an embodiment 1800, the at least one virus entry inhibitor is derived from at least one of the gp41 or gp120 components of the Human Immunodeficiency Virus. In an embodiment 1810, the at least one virus entry inhibitor includes at least one of a DNA virus entry inhibitor, or RNA virus entry inhibitor. In an embodiment 1820, the at least one virus entry inhibitor includes at least one of a double-stranded DNA virus entry inhibitor, single-stranded DNA virus entry inhibitor, double-stranded RNA virus entry inhibitor, (+) single-strand RNA virus entry inhibitor, (−) single-strand RNA virus entry inhibitor, single-strand RNA-Reverse Transcriptase virus entry inhibitor, or double-stranded DNA-Reverse Transcriptase virus entry inhibitor. In an embodiment 1830, the at least one virus entry inhibitor includes at least one of human immunodeficiency virus (HIV) type I virus entry inhibitor, HIV-type 2 virus entry inhibitor, simian immunodeficiency virus (SW) entry inhibitor, or feline leukemia virus entry inhibitor. In an embodiment 1840, the at least one virus entry inhibitor includes at least one of respiratory syncytial virus (RSV) entry inhibitor, influenza (flu) virus entry inhibitor, adenovirus entry inhibitor, rhinovirus entry inhibitor, enterovirus entry inhibitor, poliovirus entry inhibitor, rubella virus entry inhibitor, paramyxovirus entry inhibitor, herpes simplex virus type I (HSV-1) entry inhibitor, Herpes simplex virus 2 (HSV-2) entry inhibitor, rotavirus entry inhibitor, neurotropic virus entry inhibitor, coxsackie virus entry inhibitor, hepatitis virus type A entry inhibitor, hepatitis virus type B entry inhibitor, hepatitis virus type C entry inhibitor, or oncovirus entry inhibitor. In an embodiment 1850, the at least one virus entry inhibitor includes one or more of an organic or inorganic small molecule, nucleic acid, amino acid, peptide, polypeptide, protein, glycopeptide, glycoprotein, glycolipid, lipopolysaccharide, peptidoglycan, proteoglycan, lipid, metalloprotein, liposome, or carbohydrate. In an embodiment 1860, the at least one virus entry inhibitor includes at least one of maraviroc, enfuvirtide, T-22, T-2, AMD-070, BlockAide/CR, BMS 806, KRH-1636, ONO-4128, Pro-140, Pro-542, SCH-D, T-1249, TAK-220, TAK-652, TNX-355, TAK-779, palivizumab, vicriviroc, aplaviroc, AK605, or TAK-779. In an embodiment 1870, one or more of the at least one virus entry inhibitor or viral-replication modulator includes at least one antibody.

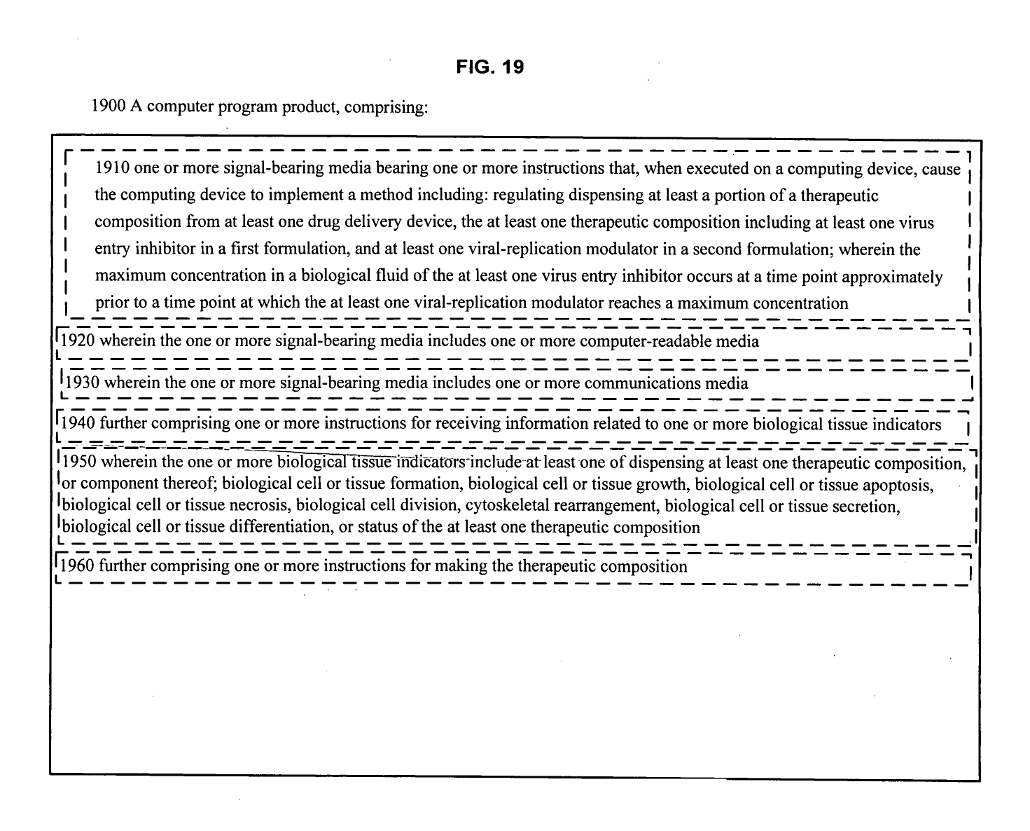

As indicated in FIG. 19, in an embodiment 1900, a computer program product comprises 1910 one or more signal-bearing media bearing one or more instructions that, when executed on a computing device, cause the computing device to implement a method including: regulating dispensing at least a portion of a therapeutic composition from at least one drug delivery device, the at least one therapeutic composition including at least one virus entry inhibitor in a first formulation, and at least one viral-replication modulator in a second formulation; wherein the maximum concentration in a biological fluid of the at least one virus entry inhibitor occurs at a time point approximately prior to a time point at which the at least one viral-replication modulator reaches a maximum concentration. In an embodiment 1920, the one or more signal-bearing media includes one or more computer-readable media. In an embodiment 1930, the one or more signal-bearing media includes one or more communications media. In an embodiment 1940, the computer program product further comprises one or more instructions for receiving information related to one or more biological tissue indicators. In an embodiment 1950, the one or more biological tissue indicators include at least one of dispensing at least one therapeutic composition, or component thereof; biological cell or tissue formation, biological cell or tissue growth, biological cell or tissue apoptosis, biological cell or tissue necrosis, biological cell division, cytoskeletal rearrangement, biological cell or tissue secretion, biological cell or tissue differentiation, or status of the at least one therapeutic composition. In an embodiment 1960, the computer program product further comprises one or more instructions for making the therapeutic composition.

### PROPHETIC EXAMPLES

### Example 1

### Composition Comprising Maraviroc and Zidovudine

An oral therapeutic composition for prophylactic treatment of a viral infection is prepared containing a virus entry inhibitor that modulates (e.g., inhibits or reduces) entry of the virus into a mammalian cell and a viral-replication modulator that modulates the activity of viral reverse transcriptase. In this example, the virus is the human immunodeficiency virus (HIV). The virus entry inhibitor is maraviroc (4,4-difluoro-N-{(1S)-3-[exo-3-(3-isopropyl-5-methyl-4H-1,2,4-triazol-4-yl)-8-azabicyclo[3.2.1]oct-8-yl]-1-phenylpropyl}cyclohexanecarboxamide; C₂₉H₄₁F₂N₅O; molecular weight of 513.67), a CCR5 co-receptor antagonist. The viral-replication modulator is zidovudine (3′-azido-3-deoxythymidine; C₁₀H₁₃N₅O₄; molecular weight of 267.24), a pyrimidine nucleoside analog reverse transcriptase inhibitor. A composition containing maraviroc and emtricitabine is formulated for oral administration.

The oral therapeutic composition containing maraviroc and zidovudine is formulated to provide immediate release of maraviroc followed by extended release of maraviroc and zidovudine. As such, the therapeutic composition includes an immediate release component containing maraviroc and an extended release component containing maraviroc and zidovudine. The immediate release component is formulated to produce a Cₘₐₓ (maximum serum concentration in the serum) for maraviroc within about 0.5 to about 2 hours after oral administration, while the extended release component is formulated to produce a Cₘₐₓ for zidovudine and additional maraviroc at a time point, e.g., 3-8 hours, after initiation of release from the immediate release component. In general, the Cₘₐₓ for the extended release component is achieved no earlier than three hours and no later than eight hours after initiation of release from the immediate release component; however, in an embodiment Cₘₐₓ is achieved with the extended release component in a shorter or longer period of time.

The oral composition includes an immediate release component containing maraviroc and appropriate excipients to provide immediate release of maraviroc upon oral administration. The immediate release component of the oral therapeutic composition includes a mixture of ingredients that breaks down quickly after administration to release the maraviroc, such as discrete layers, pellets or granules that are mixed in with or compressed with the extended release component. For example, maraviroc in combination with microcrystalline cellulose, dibasic calcium phosphate (anhydrous), sodium starch glycolate, or magnesium stearate is an immediate release formulation. The percentage of maraviroc in the immediate release component includes from about 50% to 80% by weight of the immediate release formulation.

The oral composition further includes an extended release component containing zidovudine and additional maraviroc. An extended release component of the oral therapeutic composition includes a mixture of ingredients formulated to provide delayed release of zidovudine and of additional maraviroc, such as discrete layers, pellets or granules that are mixed in with or compressed with the immediate release maraviroc. An example of an extended release formulation for zidovudine includes zidovudine in combination with polyethylene glycol 8000, hydroxypropymethylcellulose, and Eudgragit RS 30D. The percentage of ziduvudine in the extended release component includes from about 50% to 80% by weight of the extended release formulation. The additional maraviroc is co-formulated in granules with the zidovudine. Alternatively, the additional maraviroc is formulated into separate granules with extended release properties. An example of an extended release formulation for maraviroc includes maraviroc in combination with ethylcellulose, polyox, and hydroxypropylmethylcellulose. The percentage of maraviroc in the extended release component is from about 50% to 80% by weight of the extended release formulation.

In general, the overall amount of maraviroc in each dosing unit of the oral composition includes from about 150 to 1200 mg of maraviroc, depending upon the desired daily dosage, the dosing schedule and other medications taken in conjunction with the oral composition. The amount of ziduvodine in each dosing unit of the oral composition includes from about 100 to 600 mg of ziduvodine. A dosing unit includes a single pill taken once daily, multiple pills taken once daily, a single pill taken multiple times per day, or multiple pills taken multiple times per day. The amount of maraviroc in the immediate release component includes from about 20% to about 70% of the total maraviroc in the dosing unit. The amount of maraviroc in the extended release component of the oral composition includes from about 30% to about 80% of the total maraviroc in the dosing unit. In general, the amount of maraviroc in the immediate release and extended release components account for 100% of the desired maraviroc per dosing unit. As an example, the oral therapeutic composition includes one pill, taken twice daily, that includes 300 mg of maraviroc and 300 mg of ziduvodine per pill. The immediate release component of the oral composition contains 50% of the total maraviroc or 150 mg. The extended release component of the oral composition contains the other 50% of the total maraviroc and 100% of the ziduvodine, either formulated together or in separate extended release layers, pellets or granules.

Immediate release and extended release pellets, for example, are further formulated into a single dosing unit by blending the pellets with silicified microcrystalline cellulose, lactose monohydrate povidone, croscarmellose, and magnesium stearate and then compressing the admixture into a single tablet using a rotary tablet press. Alternatively, immediate release and extended release pellets can be combined into a hard or soft gelatin capsule in the absence of additional excipients.

### Example 2

### Composition Comprising Maraviroc and Emtricitabine

An oral therapeutic composition for prophylactic treatment of a viral infection is prepared containing at least one virus entry inhibitor that modulates entry of the virus into a mammalian cell and at least one viral-replication modulator that modulates the activity of viral reverse transcriptase. In this example, the virus is the human immunodeficiency virus (HIV). The at least one virus entry inhibitor is maraviroc (4,4-difluoro-N-{(1S)-3-[exo-3-(3-isopropyl-5-methyl-4H-1,2,4-triazol-4-yl)-8-azabicyclo[3.2.1]oct-8-yl]-1-phenylpropyl}cyclohexanecarboxamide; C₂₉H₄₁F₂N₅O; molecular weight of 513.67), a CCR5 co-receptor antagonist. The at least one viral-replication modulator is emtricitabine (5-fluoro-1-(2R,5S)-[2-(hydroxymethyl)-1,3-oxathiolan-5-yl]cytosine; C₈H₁₀FN₃O₃S; molecular weight of 247.24), a nucleoside analog reverse transcriptase inhibitor. A composition containing maraviroc and emtricitabine is formulated for oral administration. The composition is formulated to provide immediate release of the maraviroc followed by extended release of maraviroc and the emtricitabine.

The oral therapeutic composition including maraviroc and emtricitabine is in a solid dosage form of one or more tablets (e.g., a bilayer tablet). See, for example, Patra, et al., Acta Pharm., vol. 57, pp. 479-489 (2007), which is incorporated herein by reference. Alternatively, the oral therapeutic composition is in a solid dosage form of one or more of a hard or soft gelatin capsule. The composition is taken by a subject or administered to a subject for prophylactic treatment prior to or immediately following exposure to the HIV virus. Prophylactic treatment is initiated prior to or immediately following one or more possible exposure events that are likely to increase the risk of infection in the subject. In the case of HIV, for example, sexual contact, handling of, or exposure to infectious bodily fluids increases the risk of infection. In some aspects, prophylactic treatment is initiated prior to traveling to a location known to have high viral infection rates.

Since all individuals may be potentially at risk of contracting HIV, any individual should be considered a possible subject for prophylactic treatment. Furthermore, since certain individuals may remain asymptomatic for a time following HIV infection, such individuals should also be considered possible subjects for prophylactic treatment in order to prevent the onset of symptoms or transmission of the virus.

The oral therapeutic composition is taken by a subject or administered to a subject on a periodic basis. For example, tablets or capsules containing maraviroc and emtricitabine are administered at least once daily, over the course of about 21 days to about 35 days. Prophylactic treatment is initiated days to hours prior to anticipated virus exposure. The treatment course or regimen includes from about 1 day to about 35 days; from about 1 day to about 28 days; from about 1 day to about 21 days; from about 1 day to about 14 days; from about 1 day to about 7 days; from about 14 days to about 35 days; from about 14 days to about 28 days; from about 14 days to about 21 days; from about 21 days to about 35 days; from about 21 days to about 28 days; from about 28 days to about 35 days; or any length of time therebetween or greater. Optionally, serologic tests are performed 1 day, 1 week, 2 weeks, 4 weeks, 3 months, 6 months, and/or 1 year following suspected exposure to determine whether the subject is infected with the virus.

Each dose of the oral therapeutic composition containing maraviroc and emtricitabine includes about 600 mg of maraviroc and about 200 mg of emtricitabine and is administered to a subject once daily. Alternatively, the combination of maraviroc and emtricitabine is administered as two or more tablets or capsules, two or more times per day over the course of treatment. Tablets or capsules containing a smaller dose of maraviroc, emtricitabine (e.g., 10 mg/kg), or both are utilized for reducing side-effects or for small subjects, for example, pediatric subjects, as taught by Saez-Llorens. See, e.g., Saez-Llorens, et al., Pediatrics 121:e827-e835 (2008), which is incorporated herein by reference. As such, the oral therapeutic composition intended for administration at least once daily contains 1200 mg maraviroc; as an immediate release component of the composition as part of a coating, an outer layer, and or one or more layers of a multilayered tablet with one or more excipients such as water soluble polysaccharide gums, water soluble hydroxyalkylcelluloses, other cellulose polymers, gelatin, glucose, saccharides, or povidone; and 200 mg emtricitabine; as an inner core or one or more layers of a multilayered tablet.

The immediate release component of the composition is formulated to immediately release the virus entry inhibitor, e.g., maraviroc, upon ingestion of the solid dosage form. The proportion of maraviroc in the immediate release component of the composition is up to about 1%, up to about 5%, up to about 10%, up to about 20%, up to about 30%, up to about 40%, up to about 50%, up to about 75%, or up to about 100% of the total maraviroc in the composition.

The extended release component of the composition is formulated to provide controlled release of at least one component (e.g., emtricitabine) to maintain therapeutic blood or tissue levels of the component for a prolonged period of time. For example, the extended release component includes a formulation that is a diffusion system, a dissolution system, an osmotic system, a swelling system, an erosion controlled system, a stimulated controlled release system, or a combination thereof A diffusion system, for example, includes a reservoir in which maraviroc and emtricitabine are encapsulated by a membrane barrier composed of one or more inert polymers. Alternatively, the diffusion system includes a matrix in which maraviroc and emtricitabine are uniformly dissolved or dispersed in an inert polymeric matrix. Common examples of inert polymers for use as a membrane barrier or a matrix include hardened gelatin, methyl- or ethylcellulose, polyhydroxy-methyacrylate, polyvinylacetate, polyethylene, methylcellulose, hydroxypropyl-methylcellulose, carnauba wax, glyceryl tristearate, or combinations thereof. The release rates of maraviroc and emtricitabine in the diffusion system are dependent upon the diffusion rates of the agents through the membrane barrier or the polymeric matrix.

The oral therapeutic composition includes a multilayered tablet in which the outer layer contains maraviroc formulated for immediate release and the inner core contains maraviroc and emtricitabine formulated for extended release. The immediate release component including maraviroc and one or more of a filler, binder, disintegrant or lubricant and the extended release component including maraviroc, emtricitabine, one or more polymer, and one or more of a filler, binder, disintegrant or lubricant are compressed into a single, multilayered tablet using standard tableting procedures.

In general, the inactive ingredients or excipients included in the oral therapeutic composition of maraviroc and emtricitabine and other drug dosing combinations described here are approved for use in human subjects by the U.S. Food & Drug Administration (FDA) and are listed in either the United States Pharmacopeia (USP) or National Formulary (NF) for products sold in the United States, of the European Pharmacopeia (EP) for products sold in Europe.

### Example 3

Composition Comprising Maraviroc, Tenofovir, and Atazanavir

An oral therapeutic composition for prophylactic treatment of a viral infection is prepared containing at least one virus entry inhibitor that modulates entry of the virus into a mammalian cell, at least one viral-replication modulator that modulates the activity of viral reverse transcriptase, and at least one viral-replication modulator that modulates the activity of viral protease. In this example, the virus includes the human immunodeficiency virus (HIV). The at least one virus entry inhibitor includes maraviroc (4,4-difluoro-N-{(1S)-3-[exo-3-(3-isopropyl-5-methyl-4H-1,2,4-triazol-4-yl)-8-azabicyclo[3.2.1]oct-8-yl]-1-phenylpropyl}cyclohexanecarboxamide; C₂₉H₄₁F₂N₅O; molecular weight of 513.67), a CCR5 co-receptor antagonist. At least one viral-replication modulator includes tenofovir (9-[(R)-2-[[bis[[(isopropoxycarbonyl)oxy]methoxy]phosphinyl]methoxy]propyl]adenine fumarate (1:1); C₁₉H₃₀N₅O₁₀P.C₄H₄O₄; molecular weight of 635.52), a nucleotide analog reverse transcriptase inhibitor. Another viral-replication modulator includes atazanavir (3S,8S,9S,12S)-3,12-bis(1,1-dimethylethyl)-8-hydroxy-4,11-dioxo-9-(phenylmethyl)-6-[[4-(2-pyridinyl)phenyl]methyl]-2,5,6,10,13-pentaazatetradecanedioic acid dimethyl ester, sulfate (1:1); C₃₈H₅₂N₆O₇.H₂SO₄; molecular weight of 802.9 (sulfuric acid salt)), an inhibitor of HIV protease.

A composition containing maraviroc, tenofovir and atazanavir is formulated for oral administration. The therapeutic composition is formulated to provide immediate release of maraviroc, followed by extended release of tenofovir and atazanavir.

The oral therapeutic composition including maraviroc, tenofovir and atazanavir is a solid dosage form of one or more tablets. Alternatively, the oral therapeutic composition is a solid dosage form of one or more of a hard or soft gelatin capsule. The composition is taken by a subject or administered to a subject for prophylactic treatment prior to or immediately following exposure to the HIV virus. Prophylactic treatment can be initiated prior to or immediately following one or more possible exposure events that are likely to increase the risk of infection in the subject. In the case of HIV, sexual contact, handling of, or exposure to infectious bodily fluids increases the risk of infection. In some aspects, prophylactic treatment is initiated prior to traveling to a location known to have high HIV infection rates.

The oral therapeutic composition is taken by a subject or administered to a subject on a periodic basis. For example, tablets or capsules containing maraviroc, tenofovir and atazanavir can be administered at least once daily, over the course of about 21 days to about 35 days. Prophylactic treatment is initiated days to hours prior to anticipated viral exposure. The treatment course or regimen includes from about 1 day to about 35 days; from about 1 day to about 28 days; from about 1 day to about 21 days; from about 1 day to about 14 days; from about 1 day to about 7 days; from about 14 days to about 35 days; from about 14 days to about 28 days; from about 14 days to about 21 days; from about 21 days to about 35 days; from about 21 days to about 28 days; from about 28 days to about 35 days; or any length of time therebetween or greater. Serologic tests can be performed 1 day, 1 week, 2 weeks, 4 weeks, 3 months, 6 months, and/or 1 year following suspected exposure to determine whether the subject is infected with the virus.

Each dose of the oral therapeutic composition containing maraviroc, tenofovir and atazanavir includes about 600 mg of maraviroc, about 300 mg of tenofovir, and about 400 mg of atazanavir and is administered to a subject once daily. Alternatively, the combination of maraviroc, tenofovir and atazanavir as two or more tablets or capsules is administered one or more times per day over the course of treatment. Tablets or capsules containing a smaller dose of maraviroc, tenofovir, and or atazanavir are utilized for reducing side-effects or for small subjects such as, for example, pediatric subjects. For example, published guidelines for dosing pediatric subjects with atazanavir is weight dependent with a 150 mg dose recommended for weights below 25 kilogram and up to a 400 mg dose recommended for weights above 39 kilograms (U.S. Food & Drug Administration; “Guidelines for the Use of Antiretroviral Agents in Pediatric HIV Infection” February 2009). As such, the oral therapeutic composition intended for administration at least once daily contains an amount of maraviroc ranging from about 10 mg to about 1200 mg, an amount of tenofovir ranging from about 10 mg to about 300 mg, and an amount of atazanavir of 10 mg to about 400 mg. The maximum daily dose of atazanavir is lowered to about 300 mg by adding up to about 100 mg of ritonavir to the oral therapeutic composition. In an embodiment, tablets or capsules containing larger doses of maraviroc, tenofovir, and or atazanavir are also generated.

The oral therapeutic composition containing maraviroc, tenofovir and atazanavir includes at least one pharmaceutically-acceptable carrier or excipient, such as one or more of fillers, binders, lubricants, disintegrants, or combinations thereof, common examples of which have been described herein. In some instances, a single excipient can have multiple functionalities in the composition. For example, the excipients can also contribute to the immediate and extended release properties of the composition.

The immediate release component of the oral therapeutic composition is formulated for immediate release of maraviroc upon ingestion of the solid dosage form. The proportion of maraviroc in the immediate release component of the therapeutic composition can be up to about 1%, up to about 5%, up to about 10%, up to about 20%, up to about 30%, up to about 40%, up to about 50%, up to about 75%, or up to about 100% of the total maraviroc in the oral therapeutic composition. The immediate release component containing maraviroc is part of a coating, an outer layer, or one or more layers of a multilayered tablet, and contains maraviroc in combination with at least one excipient, such as water soluble polysaccharide gums, water soluble hydroxyalkylcelluloses, other cellulose polymers, gelatin, glucose, saccharides, or povidone. As an example, maraviroc is formulated in hydroxypropyl methylcellulose (HPMC) in the presence of glycerol and incorporated into a film coating the outer surface of the solid dosage form.

The extended release component of the therapeutic composition is formulated to provide controlled release of the maraviroc, tenofovir and atazanavir, or to maintain therapeutic blood or tissue levels of the agents for a prolonged period of time. The extended release component of the therapeutic composition includes an inner core, one or more layers of a multilayered tablet, or encapsulated particles within a larger tablet or capsule. The extended release component includes a formulation that is a diffusion system, a dissolution system, an osmotic system, a swelling system, an erosion controlled system, a stimulated controlled release system, or a combination thereof. A dissolution system, for example, includes microcapsules of maraviroc, tenofovir and atazanavir coated with slowly soluble polymers and incorporated into a larger oral dosage form. Release of maraviroc, tenofovir and atazanavir from the microcapsules can be controlled by adjusting the size of the microcapsules, the thickness of the coating materials, or the diffusivity of the core materials. Common examples of coating materials include gelatin, carnauba wax, shellac, cellulose acetate phthalate, and cellulose acetate butyrate. The thickness of the coat is varied from less than about 1 μm to about 200 μm by increasing the amount of coating material relative to the amount of active agent. A spectrum of different coating thicknesses is used to provide continuous and extended release of the agents from the microcapsules.

The microcapsules containing maraviroc, tenofovir and atazanavir for extended release are compressed into tablets or filled into capsules. The tablets or capsules are further coated with a film coating containing maraviroc formulated for immediate release. Optionally, the film coating also includes tenofovir, atazanavir, or a combination thereof formulated for immediate release. The proportion of tenofovir and or atazanavir in the film coating includes up to about 1%, up to about 5%, up to about 10%, up to about 20%, up to about 30%, up to about 40%, up to about 50%, up to about 75%, or up to about 100% of the total tenofovir and or atazanavir in the solid dosage form.

### Example 4

### Composition Containing Palivizumab and Modulator of RSV Replication

An injectable therapeutic composition for prophylactic treatment of a viral infection in a subject is prepared containing at least one virus entry inhibitor that modulates entry of the virus into a mammalian cell, and at least one viral-replication modulator. In this example, the virus includes the respiratory syncytial virus (RSV). The at least one virus entry inhibitor includes palivizumab, a humanized monoclonal antibody directed against an epitope in the A antigenic site of the F protein of RSV that blocks viral entry. The at least one viral-replication modulator includes at least one low molecular weight inhibitor of RSV replication, the substituted benzimidazole TMC353121 See, for example, Bonfanti, et al., Abstract, J. Med. Chem. 51:875-896 (2008), which is incorporated herein by reference. A composition containing palivizumab and TMC353121 is formulated for intramuscular injection for prophylactic treatment of RSV. The formulation provides for immediate release of palivizumab, and extended release of TMC353121.

The injectable therapeutic composition comprising palivizumab and TMC353121 is administered to a subject for prophylactic treatment prior to or immediately following exposure to RSV. Infection with RSV usually causes mild, cold-like symptoms, but in a subset of subjects it can cause severe and life-threatening disease. At particular risk for severe complications of RSV infection are premature infants, infants born with lung or heart disease, infants with low birth weight, infants with a family history of asthma, infants exposed to tobacco smoke and other air pollutants, or a combination thereof. Also at risk are adults with chronic heart disease, chronic lung disease, or compromised immune systems and adults aged 65 or older, particularly those residing in a long-term care facility or participating in other senior day-care programs.

The initiation and continuation of prophylactic treatment of subjects at risk for RSV is dependent on the location and time of year. In the northern hemisphere, for example, the RSV season typically begins in November and lasts through April, but can vary from year to year. Information regarding the reported seasonal incidence of RSV infection can be obtained from the National Respiratory and Enteric Virus Surveillance System. See, for example, Center for Disease Control and Prevention, Atlanta, Ga., available on the world wide web at cdc.gov/surveillance/nrevss/rsv/state.html (the content of which is incorporated herein by reference), or from a state or local health department.

Prophylactic treatment with the injectable therapeutic composition containing palivizumab and TMC353121 is initiated prior to the onset of the RSV season. Alternatively, prophylactic treatment is initiated just prior to entering an environment prone to increased infection rate such as, for example, a child care or elder care setting.

The injectable therapeutic composition containing palivizumab and TMC353121 is taken by a subject or administered to a subject on a periodic basis. For example, the subject receives an intramuscular injection of the composition on a monthly basis throughout the RSV season. Prophylactic treatment is initiated days to hours prior to anticipated RSV exposure. Prophylactic treatment is initiated about 1 to about 30 days prior to anticipated viral exposure. The prophylactic treatment course is from about 1 month to about 12 months, from about 1 month to about 10 months, from about 1 month to about 8 months, from about 1 month to about 6 months, from about 1 month to about 4 months, from about 3 months to about 10 months, from about 3 months to about 8 months, from about 3 months to about 6 months or any length of time therebetween or greater.

Each dose of the injectable therapeutic composition containing palivizumab and TMC353121 includes about 100 mg of palivizumab and about 3000 mg of TMC353121 and is administered to a subject once monthly. Alternatively, the combination of palivizumab and TMC353121 is administered as two or more injections per dosing or one or more injections two or more times per month over the course of treatment. Smaller doses of palivizumab, TMC353121, or both are utilized for prophylactic treatment of small subjects such as pediatric subjects. Published guidelines for dosing a pediatric subject with palivizumab recommend a dose of 15 mg/kg (see, e.g., Mejias & Ramilo, Biologics, Vol 2:433-439 (2008), which is incorporated herein by reference). As such, the injectable therapeutic composition intended for administration at least once monthly contains an amount of palivizumab ranging from about 10 mg to about 100 mg and an amount of TMC353121 ranging from about 30 mg to about 3000 mg. Injectable formulations contain larger doses of palivizumab, TMC353121, or both.

The injectable therapeutic composition containing palivizumab and TMC353121 includes at least one pharmaceutically acceptable carrier or excipient such as antimicrobial agents, buffers, antioxidants, tonicity agents, or cryoprotectants and lyoprotectants. Antimicrobial agents in bacteriostatic or fungistatic concentrations are added to preparations of multiple dose preparations to prevent possible microbial growth inadvertently introduced during withdrawal of a portion of the vial contents. Common examples of antimicrobial agents include phenylmercuric nitrate, thimerosal, benzethonium chloride, benzalkonium chloride, phenol, cresol, and or chlorobutanol. Buffers are used to stabilize a solution against chemical or physical degradation. Common acid salts used as buffers include citrates, acetates and phosphates.

Antioxidants are used to preserve products against oxidation. Common examples of antioxidants include sodium bisulfite, ascorbic acid, and salts, thereof. Tonicity agents are used to ensure that injected material is isotonic with physiological fluids. Common examples of tonicity agents include electrolytes and monosaccharides or disaccharides. Cryoprotectants and lyoprotectants are additives that protect active ingredients from damage due to the freeze-drying process. Common cryoprotectant and lyoprotectant agents include sugars, amino acids, polymers, and polyols.

The immediate release component of the injectable therapeutic composition is formulated to immediately release palivizumab upon injection. The proportion of palivizumab in the immediate release component of the therapeutic composition can be up to about 1%, up to about 5%, up to about 10%, up to about 20%, up to about 30%, up to about 40%, up to about 50%, up to about 75%, or up to about 100% of the total palivizumab in the injectable therapeutic composition. The immediate release component containing palivizumab can be an aqueous fraction of the injectable therapeutic composition, for example, composed of water and chlorine for osmotic balance, and amino acids to stabilize the protein based antibody.

The extended release component of the therapeutic composition is formulated to provide extended release of TMC353121, and to maintain therapeutic blood or tissue levels of the agents for a prolonged period of time. For example, the extended release component (TMC353121) includes a formulation that is a diffusion system, a dissolution system, an osmotic system, a swelling system, an erosion controlled system, a stimulated controlled release system, or a combination thereof and can form a depot of drug at the site of injection. A dissolution system, for example, can include microcapsules of palivizumab and TMC353121 coated with slowly soluble polymers. Release of palivizumab and TMC353121 from the microcapsules can be controlled by adjusting the size of the microcapsules, the thickness of the coating materials, and the diffusivity of the core materials. Common examples of coating materials include gelatin, carnauba wax, shellac, cellulose acetate phthalate, and cellulose acetate butyrate. The thickness of the coat is varied from less than about 1 μm to about 200 μm by increasing the amount of coating material relative to the amount of active agent. A spectrum of different coating thicknesses is used to provide continuous and extended release of the agents from the microcapsules.

In an embodiment, palivizumab and TMC353121 are formulated together into microcapsules. In an embodiment, palivizumab and TMC353121 are formulated into separate microcapsules to enable adjustments to the release behavior of each agent.

The immediate release component containing palivizumab is combined with the extended release microcapsules containing palivizumab and TMC353121. Palivizumab and appropriate excipients are lyophilized and the powder mixed with extended release microcapsules into an injection vial. Just prior to intramuscular injection, sterile water is added to the mixture to solubilize the palivizumab and suspend the microcapsules.

### Example 5

### Composition Comprising an Entry Blocking Peptide and Zanamivir

An inhaled therapeutic composition for prophylactic treatment of a viral infection in a subject is prepared containing at least one virus entry inhibitor that modulates entry of the virus into a mammalian cell and at least one viral-replication modulator. In this example, the virus includes one of several influenza viruses. The at least one virus entry inhibitor includes at least one entry blocking peptide, for example, one or more peptides that bind hemagglutinin and prevent attachment of the influenza virus to mammalian cells. See, for example, Jones, et al., J. Virol. 80:11960-11067 (2006); or Jeon, et al., J. Biol. Chem., vol. 279, no. 46, pp. 48410-0.48419, (2004), each of which is incorporated herein by reference. The at least one viral-replication modulator includes zanamivir (5-(acetylamine)-4-[(aminoiminomethyl)-amino]-2,6-anhydro-3,4,5-trideoxy-D-glycero-D-galactonon-2-enonic acid; C₁₂H₂₀N₄O₇; molecular weight of 332.3), a neuramidase inhibitor affecting release of viral particles from the host cell. A composition containing the entry blocking peptide and zanamivir is formulated for inhaled administration for prophylactic treatment of influenza. The formulation provides for immediate release of the entry blocking peptide and extended release of both the entry blocking peptide and zanamivir.

The inhaled therapeutic composition is administered to a subject for prophylactic treatment prior to or immediately following exposure to influenza virus. The influenza virus includes various strains associated with common flu symptoms (e.g., influenza A (H1N1, H3N2) and influenza B strains) as well as strains associated with the more deadly avian influenza (e.g., H5N1).

The initiation and continuation of prophylactic treatment of a subject depends on the time of year and location of the subject. In the United States, for example, the flu season typically begins in November and lasts through March, but can vary from year to year. Information regarding local, national and international influenza infection rates can be obtained from Flu Activity & Surveillance (Center for Disease Control and Prevention, Atlanta, Ga.; on the worldwide web at: cdc.gov/flu/weekly/fluactivity.htm, the content of which is incorporated herein by reference), or from a state or local health department. Information regarding the current incidence of avian influenza can be obtained from the World Health Organization (on the worldwide web at: who.int/csr/disease/avian_influenza/en/index.html, the content of which is incorporated herein by reference).

In an embodiment, prophylactic treatment with the inhaled therapeutic composition containing the entry blocking peptide and zanamivir is initiated prior to the onset of the flu season. In an embodiment, prophylactic treatment is initiated just prior to entering an environment prone to or known to have an increased risk of infection such as a child care facility, a school, a hospital, a bus, a train, an airplane, or other crowded facility. In the case of avian influenza, prophylactic treatment is initiated prior to travel to a part of the world known to have increased infection rates, such as Asia.

The inhaled therapeutic composition including at least the entry blocking peptide and zanamivir is taken by a subject or administered to a subject on a periodic basis. For example, the inhaled therapeutic composition is administered on a daily basis throughout the flu season. In an embodiment, prophylactic treatment is initiated 1 to 30 days prior to anticipated viral exposure due to approaching flu season, travel to an area with increased infection risk, or a combination thereof. The treatment course or regimen includes from about 1 day to about 180 days; from about 1 day to about 150 days; from about 1 day to about 120 days; from about 1 day to about 90 days; from about 1 day to about 60 days; from about 1 day to about 30 days, or any length of time therebetween or greater. In an embodiment, prophylactic treatment is continued for a number of days after leaving a potentially infectious area. The incubation period for development of influenza symptoms ranges from about 1 day to about 5 days.

Each dose of the inhaled therapeutic composition containing the entry blocking peptide and zanamivir includes about 20 mg of the entry blocking peptide and about 10 mg of zanamivir and is administered to a subject once daily. Alternatively, the combination of the entry blocking peptide and zanamivir is administered as two or more inhalation puffs per dosing or one or more inhalation puffs two or more times per day over the course of treatment. Smaller doses of the entry blocking peptide, zanamivir, or both are utilized for prophylactic treatment of small subjects such as, for example, pediatric subjects. As such, the inhaled therapeutic composition intended for administration at least once daily contains an amount of the entry blocking peptide ranging from about 0.1 mg to about 20 mg and an amount of zanamivir ranging from about 0.1 mg to about 10 mg.

The inhaled therapeutic composition containing the entry blocking peptide and zanamivir include one or more pharmaceutically-acceptable carriers or excipients depending upon whether a nebulizer, dry powder inhaler, nasal inhaler, or pressurized metered dose inhaler (pMDI) is used for delivery. Dry powder inhalers and pMDIs provide solid drug suspended or dissolved in a nonpolar volatile propellant or in a dry powder mix that is fluidized when the subject inhales. The particle size ideally ranges from about 1 μm to about 10 μm for efficient delivery of the therapeutic composition to multiple levels of the pulmonary airways. Excipients are added to enhance the physical and or chemical stability, mechanical properties, or dissolution and permeation properties of the active ingredients. Excipients can also serve to provide bulk to the inhaled dose. A common excipient is lactose. Other excipients include mannitol, glucose, phosphatidyl choline and cholesterol (as part of liposomal formulation), antioxidants (e.g., ascorbic acid) or dispersing agents (e.g., sorbitan trioleate, oleyl alcohol, oleic acid, and lecithin) In some instances, a single excipient can have multiple functionalities in the composition. For example, the excipients also contribute to the immediate and extended release properties of the composition.

The immediate release component of the inhaled therapeutic composition is formulated to immediately release the entry blocking peptide, upon inhalation. The proportion of the entry blocking peptide in the immediate release component of the therapeutic composition can be up to about 1%, up to about 5%, up to about 10%, up to about 20%, up to about 30%, up to about 40%, up to about 50%, up to about 75%, or up to about 100% of the total amount of entry blocking peptide in the inhaled therapeutic composition. The immediate release component containing the entry blocking peptide can be dry powder particles formulated with or without excipients to a particle size ranging from about 1 μm to about 10 μm. Examples of excipients used in dry powder formulations of other inhaled peptides, e.g., inhaled insulin, include sodium citrate, mannitol, glycine and sodium hydroxide.

The extended release component of the therapeutic composition is formulated to provide extended release of the entry blocking peptide and zanamivir, to maintain therapeutic blood or tissue levels of the agents for a prolonged period of time. The entry blocking peptide and zanamivir can be formulated for extended release in one or more colloidal drug carriers such as microparticles, nanoparticles, macromolecular complexes (e.g., lipoproteins), liposomes, or niosomes. Liposomes, for example, can be used to encapsulate therapeutic agents for inhaled drug delivery and are generated by self-assembly of phospholipids, aminolipids, sphingolipids, glycosphingolipids, diacylglycerols, triglycerides, sterols, long-chain dialkyl dimethyl ammonium compounds, or a combinations thereof into lipid bilayer vesicles (see, e.g., Wittgen, et al., Clin. Cancer Res. 13:2414-2421 (2007) which is incorporated herein by reference). For example, liposomes are formed in the presence of an aqueous solution containing the entry blocking peptide, zanamivir, or both, by any of a variety of methods including hydration of lipid films, solvent injection, reverse-phase evaporation, sonication, extrusion, high pressure/homogenization, microfluidization, detergent dialysis, calcium-induced fusion of small liposome vesicles, or ether-infusion methods. Sterols added to the liposomes increase the stability of the liposomal bilayers. Lipids possessing a positive or negative change, for example, phosphatidyl-ethanolamine, gangliosides or phosphatic acid are used to render the appropriate charge to the liposomes and to increase the size of the aqueous compartments. Mixtures of lipids are used to render the liposomes more fluid or more rigid and to increase or decrease permeability characteristics.

The entry blocking peptide and zanamivir are formulated together into the same liposomes or formulated separately into liposomes of the same or different composition, depending upon the desired release properties. For example, an example of a liposome composition for a peptide includes polyethylene glycol-conjugated distearyl phosphatidyl ethanolamine (DSPE-PEG2000), lysostearyl-phosphatidylglycerol (lyso-PG) and palmitoyl-oleoyl-phosphatidylcholine (POPC) (see, e.g., Hajos, et al., Int. J. Pharm. 357:286-294 (2008) which is incorporated herein by reference). In an embodiment, an example of a liposome composition for a small molecule includes dipalmitoyl phosphatidylcholine and cholesterol (see, e.g., Meers, et al., J. Antimicrob. Chemother., 61:859-868 (2008); Wittgen, et al., Clin. Cancer Res. 13:2414-2421 (2007), each of which are incorporated herein by reference).

The liposomes containing the entry blocking peptide and zanamivir are lyophilized in the presence of a cryoprotectant such as maltose, dextrose, trehalose, lactose, sucrose, or a combination thereof. The lyophilized material is broken up, sized through a series of sieves and micronized to particles ranging in size from about 0.5 μm to about 10 μm. The resulting particles of liposomes containing the entry blocking peptide and zanamivir are mixed with the dry powder immediate release component containing the entry blocking peptide generated as described above and the combination is used for dry powder inhalation. Alternatively, the liposomes containing the entry blocking peptide and zanamivir are dispersed throughout an aqueous phase such as a solution of sodium chloride (0.9%). The aqueous phase further includes solubilized entry blocking peptide as the immediate release component of the composition. The aqueous phase containing dispersed liposomes are delivered to a subject, for example, as an aqueous aerosol via nebulization.

### Example 6

### Composition Comprising Entry Blocking Peptide and Ribavirin

A transdermal therapeutic composition for prophylactic treatment of a viral infection in a subject is prepared containing at least one virus entry inhibitor that modulates entry of the virus into a mammalian cell and at least one viral-replication modulator that modulates the replication of the virus. In this example, the virus includes the hepatitis C virus (HCV). The at least one virus entry inhibitor includes one or more entry blocking peptides that inhibit HCV infection such as, for example, Cyanovirin-N (see, e.g., Helle, et al., J. Biol. Chem. 281:25177-25183 (2006) which is incorporated herein by reference). The at least one viral-replication modulator includes ribavirin (1-β-D-ribofuranosyl-1H-1,2,4-triazole-3-carboxamide; C₈H₁₂N₄O₅; molecular weight of 244.2), a nucleoside analog with antiviral activity. A composition containing the Cyanovirin-N and ribavirin is formulated as a transdermal patch for topical prophylactic treatment of HCV. The formulation provides for immediate release of Cyanovirin-N, and extended release of both Cyanovirin-N and ribavirin.

The transdermal therapeutic composition containing Cyanovirin-N and ribavirin is administered to a subject for prophylactic treatment prior to or immediately following exposure to hepatitis C. In an embodiment, prophylactic treatment is initiated prior to or immediately following one or more possible exposure events that are likely to increase the risk of infection in the subject. In the case of HCV, exposure to infectious blood or blood products increases the risk of infection and can include occupational exposure (e.g., needle sticks), exposure to inadequately sterilized needles, syringes or other medical equipment, or exposure to needles shared by drug-users, or exposure through sexual or prenatal contact. Other modes of transmission include body piercing, circumcision and tattooing with inadequately sterilized equipment. In some aspects, prophylactic treatment is initiated prior to traveling to a location known to have high viral infection rates, inadequate medical facilities, or both. For example, in some developing countries, medical facilities can use inadequately sterilized injection equipment or can not properly screen blood prior to transfusion, increasing the risk of HCV infection during a medical procedure.

The transdermal therapeutic composition comprising Cyanovirin-N and ribavirin is applied to a subject on a periodic basis. For example, the transdermal therapeutic composition is applied on a daily basis. In an embodiment, prophylactic treatment is initiated hours to days prior to anticipated viral exposure. In an embodiment, the treatment course or regimen can include from about 1 day to about 35 days; from about 1 day to about 28 days; from about 1 day to about 21 days; from about 1 day to about 14 days; from about 1 day to about 7 days; from about 14 days to about 35 days; from about 14 days to about 28 days; from about 14 days to about 21 days; from about 21 days to about 35 days; from about 21 days to about 28 days; from about 28 days to about 35 days; or any length of time therebetween or greater. Optionally, serologic tests can be performed about 2 weeks, about 4 weeks, about 3 months, about 6 months, or about 1 year following suspected exposure to determine whether the subject is infected with the virus.

Each dose of the transdermal therapeutic composition containing Cyanovirin-N and ribavirin includes about 20 mg of Cyanovirin-N and about 1200 mg of ribavirin and is administered to a subject once daily. Alternatively, the combination of Cyanovirin-N and ribavirin is administered as two or more applications once daily, or one or more applications twice or more per day over the course of treatment. Smaller doses of Cyanovirin-N, ribavirin, or both are utilized for prophylactic treatment of small subjects, such as pediatric subjects. As such, the transdermal therapeutic composition intended for administration at least once daily contains an amount of Cyanovirin-N ranging from about 0.1 mg to about 20 mg and an amount of ribavirin ranging from about 10 mg to about 1200 mg.

The transdermal therapeutic composition containing Cyanovirin-N and ribavirin can include a transdermal patch system. Examples of transdermal patch systems include membrane modulated systems, adhesive-dispersion systems, matrix dispersion systems, microreservoir systems, or combinations thereof. In a membrane modulated system, for example, the active agents are held in solution or suspension between the backing layer of the patch and a rate controlling membrane. An inert adhesive layer facilitates attachment to the skin. By comparison, in an adhesive-dispersion system, the active agents are incorporated into and diffuse out of the adhesive layer. Other transdermal delivery systems can be used to deliver the therapeutic composition including topical cream, gel or pastes, suppository, iontophoresis, electrophoresis, microneedles, and others.

The transdermal therapeutic composition containing Cyanovirin-N and ribavirin also includes a number of inactive ingredients or excipients such as, for example, release-rate controlling polymers, penetration enhancers, and adhesives. Release-rate controlling polymers are used to control the rate of release of Cyanovirin-N and ribavirin from the transdermal patch. Common examples of polymers include polyvinyl alcohol, polyoxyethylene, poly(hydroxyethyl methacrylate), polyvinylpyrrolidone, polypropylene, polyesters, ethylene-vinyl acetate copolymer, polyisoprene, and the like. Penetration enhancers are optionally added to the transdermal composition to enhance penetration of Cyanovirin-N and ribavirin across the dermis. Common examples of penetration enhancers include solvents (e.g., water, alcohols, alkyl methyl sulfoxides, pyrrolidones, dimethyl formamide, acetone), amphiphiles (e.g., amino acids, surfactants, fatty acids), clofibric acid amides, proteolytic enzymes, urea, and combinations thereof. Examples of cell permeable peptide penetration enhancers, or Protein Transduction Domains (PTDs), include antennapedia peptide, buforin, lipid membrane translocating peptide, mastoparan, HIV-TAT and transportan. In some instances, the PTD is incorporated into a recombinant fusion protein/peptide that includes the PTD and Cyanovirin-N.

In an embodiment, co-administration of the PTD and the entry blocking peptide is sufficient for enhanced transdermal delivery. For example, the transdermal patch can include a short synthetic peptide, “transdermal peptide”, that facilitates transdermal drug delivery through skin by creating a transient opening in the skin barrier and enabling macromolecular drugs to reach the systemic circulation (see, e.g., U.S. Patent Application Publication No. 2008/0305989, which is incorporated herein by reference; “transdermal peptide” is commercially available from AnaSpec, San Jose, Calif.).

Adhesives are optionally used to attach the patch to the skin and can also be used as a matrix for drug delivery. Common examples of adhesives include vinyl acetates, silicones, and polyacrylates.

The transdermal therapeutic composition is formulated to immediately release Cyanovirin-N, upon attachment of the patch to the skin. The proportion of Cyanovirin-N in the immediate release component of the therapeutic composition can be up to about 1%, up to about 5%, up to about 10%, up to about 20%, up to about 30%, up to about 40%, up to about 50%, up to about 75%, or up to about 100% of the total amount of peptide in the transdermal therapeutic composition. In this example, the immediate release component containing Cyanovirin-N is incorporated into the adhesive layer. The adhesive layer is composed of acetylate vinyl acetate copolymers or other adhesive polymer materials, and can optionally include one or more penetration enhancing excipients and other excipients.

The extended release component of the transdermal therapeutic composition is formulated to provide extended release of both the Cyanovirin-N and ribavirin, to maintain therapeutic blood or tissue levels of the agents for a prolonged period of time. The components can be formulated for extended release by inclusion, for example, in one or more reservoirs separated from the skin by a rate-controlling polymeric membrane. In an embodiment, the polymeric membrane is a microporous polypropylene material. The rate of drug release of Cyanovirin-N and ribavirin is controlled by varying the polymer composition, permeability coefficient, or thickness of the polymeric membrane and any associated adhesive layer. In some aspects, Cyanovirin-N and ribavirin are formulated in separate reservoirs with distinct release properties based on the composition of the excipients and the polymeric membrane associated with each reservoir. In an embodiment, the adhesive layer containing Cyanovirin-N for immediate release is layered with the reservoir and polymeric membrane containing Cyanovirin-N and ribavirin for extended release in an overlapping or non-overlapping configuration to form the transdermal patch.

### Example 7

### Composition Comprising Enfuvirtide and Zidovudine

An injectable therapeutic composition for prophylactic treatment of a viral infection is prepared containing a virus entry inhibitor that modulates entry of the virus into a mammalian cell and a viral-replication modulator that modulates the activity of viral reverse transcriptase. In this example, the virus includes the human immunodeficiency virus (HIV-1). The virus entry inhibitor includes enfuvirtide (linear 36 amino acid synthetic peptide; C₂₀₄H₃₀₁N₅₁O₆₄; molecular weight of 4492), an inhibitor of HIV-1 gp41 mediated fusion. Theviral-replication modulator includes zidovudine (3′-azido-3′-deoxythymidine, C₁₀H₁₃N₅O₄; molecular weight of 267.24), a nucleoside analog reverse transcriptase inhibitor. A composition containing enfuvirtide and zidovudine is formulated for intrauterine administration to the fetus of an HIV infected mother prior to the onset of labor, during labor, or delivery (including by Caesarean or surgically assisted delivery), or a combination thereof, to prevent maternal transmission of the virus to the child.

The injectable therapeutic composition including enfuvirtide and zidovudine is administered directly to the fetus of an HIV infected mother for prophylactic treatment of HIV. It is not necessary for the HIV status of the mother to be known prior to initiating therapy. In some instances, the mother knows prior to the onset of labor or delivery that she is HIV positive, and may or may not be taking antiretroviral drugs for the treatment of HIV. For example, some anti-retroviral drugs freely pass across the placenta while others, e.g., enfuvirtide, do not efficiently pass from mother to child (see, e.g., Ceccaldi, et al. Am. J. Obstet. Gynecol. 198:433e1-433e2 (2008), which is incorporated herein by reference).

In some instances, the mother does not know her HIV status at the onset of labor or delivery. If desired, a rapid point-of-care serologic test can be used to detect antibodies to HIV in the serum of the mother (see, e.g., Branson. J. Lab. Med. 27:288-295 (2003), which is incorporated herein by reference). Prophylactic treatment of the fetus with the injectable therapeutic composition is initiated regardless of whether the mother's HIV status is known for subjects living in locations in which the HIV infection rate is known to be high among the adult population.

Each dose of the injectable therapeutic composition includes amounts of enfuvirtide and zidovudine appropriate for administration to neonatal or pediatric subjects. Current dosing guidelines for pediatric subjects recommend up to 4 mg/kg of enfuvirtide and up to 6 mg/kg of zidovudine on a daily basis (U.S. Food & Drug Administration; “Guidelines for the Use of Antiretroviral Agents in Pediatric HIV Infection,” February 2009). As such, each dose of the injectable therapeutic composition contains up to about two parts enfuvirtide and up to about three parts zidovudine.

In an embodiment, the dosing is determined according to the weight of the subject. For example, an average fetus weighs about 1.32 kilograms at 30 weeks, about 2.38 kilograms at 35 weeks, and about 3.5 kilograms at 40 weeks. As such, a fetus weighing about 3 kilograms at the time that the HIV positive mother enters labor or delivery is dosed with an injectable therapeutic composition containing up to about 12 mg of enfuvirtide and up to about 18 mg of zidovudine.

The injectable therapeutic composition containing enfuvirtide and zidovudine also includes one or more pharmaceutically acceptable carriers or excipients, such as one or more of antimicrobial agents, buffers, antioxidants, tonicity agents, cryoprotectants, lyoprotectants, or combinations thereof. In some instances, a single excipient can have multiple functionalities in the composition.

In an embodiment, the injectable therapeutic composition containing enfuvirtide and zidovudine is directly injected into the fetal abdominal cavity. Injection can be performed using a 23-gauge needle and color Doppler ultrasound for guidance (see, e.g., Matsuda, et al., BJOG 111:756-757 (2004) which is incorporated herein by reference). In an embodiment, the therapeutic composition is administered to the fetus by injection into the umbilical vein or percutaneous injection using ultrasound guidance (see, e.g., David, et al., Abstract, Hum. Gene Ther. 14:353-64 (2003), which is incorporated herein by reference).

In an embodiment, the neonate continues to be treated with the injectable therapeutic composition post delivery to provide prophylactic protection until the HIV status of the child is known. The neonate is optionally periodically tested for the presence of HIV infection post delivery to determine whether continued treatment with the injectable therapeutic composition or initiation of another antiretroviral regimen is warranted. HIV DNA polymerase chain reaction (PCR) amplification is optionally used to assess the presence of specific HIV viral sequences in integrated proviral HIV DNA in the neonate's peripheral blood mononuclear cells (PBMCs). This assay has a sensitivity of up to about 40% at less than 48 hours post birth and close to about 90% sensitivity 2-4 weeks post birth. HIV RNA assays can also be used to detect extracellular viral RNA in the plasma of the neonate. This assay has a sensitivity of about 25-40% at 2-4 weeks post birth and about 90-100% sensitivity by 2-3 months. See, e.g., Working Group on Antiretroviral Therapy and Medical Management of HIV-Infected Children, Guidelines for the Use of Antiretroviral Agents in Pediatric HIV Infection, Feb. 23, 2009; pp 1-138, which is incorporated herein by reference. In certain instances, an HIV antibody assay can be used for detecting HIV infection in neonates, bearing in mind the potential transfer to the fetus of maternal HIV antibodies in an HIV positive mother.

While particular aspects of the present subject matter described herein have been shown and described, it will be apparent that, based upon the teachings herein, changes and modifications may be made without departing from the subject matter described herein and its broader aspects and, therefore, the appended claims are to encompass within their scope all such changes and modifications as are within the true spirit and scope of the subject matter described herein. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B,*and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although various operational flows are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

All publications and patent applications cited in this specification are herein incorporated by reference to the extent not inconsistent with the description herein and for all purposes as if each individual publication or patent application were specifically and individually indicated to be incorporated by reference for all purposes.

1. A drug delivery device, comprising:
a housing including at least one reservoir containing at least one therapeutic composition, the at least one reservoir configured to deliver at least a portion of the at least one therapeutic composition to at least one biological tissue,
wherein the at least one therapeutic composition includes at least one virus entry inhibitor in a first formulation; at least one viral-replication modulator in a second formulation; wherein the maximum concentration in a biological fluid of the at least one virus entry inhibitor occurs at a time point approximately prior to a time point at which the at least one viral-replication modulator reaches a maximum concentration.

2. The drug delivery device of claim 1, wherein the device is implantable.

3. The drug delivery device of claim 2, wherein the device is implanted into a subject.

4. The drug delivery device of claim 3, wherein the device is implanted into at least one of lymph node, lymph, spleen, blood vessel, blood, liver, pancreas, gastrointestinal tract, Peyer's patch, epithelial tissue, vagina, or other biological tissue.

5. The drug delivery device of claim 1, wherein the device is external to a subject.

6. The drug delivery device of claim 1, wherein the at least one reservoir further comprises one or more ports configured to allow filling or dispensing of the at least one reservoir.

7. The drug delivery device of claim 1, further comprising one or more controllable output mechanisms operably linked to the one or more outlets to control the dispensing of at least a portion of the at least one therapeutic composition from the at least one reservoir.

8. The drug delivery device of claim 7, wherein the at least one controllable output mechanism includes at least one micropump.

9. The drug delivery device of claim 7, wherein the at least one controllable output mechanism includes at least one thermal or nonthermal gate in communication with the one outlet of the at least one reservoir.

10. The drug delivery device of claim 7, further comprising at least one control circuitry configured to control the at least one controllable output mechanism.

11. The drug delivery device of claim 10, wherein the at least one control circuitry is configured to generate and transmit at least one of an electromagnetic or electrical control signal configured to control the at least one controllable output mechanism.

12. The drug delivery device of claim 10, further comprising at least one memory mechanism for storing instructions for generating and transmitting the electromagnetic control signal.

13. The drug delivery device of claim 10, wherein the at least one control circuitry is configured to control the at least one controllable output mechanism for time-release of at least a portion of the at least one therapeutic composition from the at least one reservoir.

14. The drug delivery device of claim 10, wherein the at least one control circuitry is configured for variable programming control of the at least one controllable output mechanism.

15. The drug delivery device of claim 1, further comprising at least one sensor component including one or more sensors.

16. The drug delivery device of claim 15, wherein at least one sensor includes a sensor configured to detect the presence or level of one or more biological signaling molecules.

17. The drug delivery device of claim 16, wherein the one or more biological signaling molecules include at least a portion of one or more of an organic or inorganic small molecule, nucleic acid, amino acid, peptide, polypeptide, protein, glycopeptide, glycoprotein, glycolipid, lipopolysaccharide, peptidoglycan, proteoglycan, lipid, metalloprotein, liposome, or carbohydrate, receptor, ligand, antibody, cytokine, or virus particle.

18. The drug delivery device of claim 16, wherein at least one of the one or more biological signaling molecules is indicative of a viral infection.

19. The drug delivery device of claim 16, wherein the device is configured to release at least a portion of the therapeutic composition in response to the presence or level of one or more biological signaling molecules.

20. The drug delivery device of claim 16, wherein the device is configured to release at least a portion of the therapeutic composition in response to a comparison of the levels of two or more biological signaling molecules.

21. The drug delivery device of claim 16, wherein the device is configured to release at least a portion of the therapeutic composition in response to the absence of one or more biological signaling molecules.

22. The drug delivery device of claim 16, wherein the at least one sensor configured to detect the presence or level of one or more biological signaling molecules includes one or more detection indicators.

23. The drug delivery device of claim 22, wherein the one or more detection indicators include at least one of a dye, radioactivity, fluorescence, electromagnetic energy, magnetism, or other detectable indicator.

24. The drug delivery device of claim 16, wherein at least one sensor includes a sensor configured to detect at least one quantity of the at least one therapeutic composition or a component thereof.

25. The drug delivery device of claim 16, wherein the at least one sensor is located in or proximate to the at least one reservoir.

26. The drug delivery device of claim 16, wherein the at least one sensor includes one or more detection indicators.

27. The drug delivery device of claim 16, wherein the at least one sensor configured to detect at least one quantity of the at least one therapeutic composition or a component thereof includes at least a part of the same sensor as at least one sensor configured to detect at least one biological signaling molecule.

28. The drug delivery device of claim 16, wherein the at least one sensor includes one or more of an electrochemical transducer, chemical transducer, ultrasonic transducer, optical transducer, piezoelectrical transducer, or thermal transducer.

29. The drug delivery device of claim 16, further comprising at least one transmitter, capable of communicating the presence or level of one or more biological signaling molecules to at least one computer system.

30. The drug delivery device of claim 16, further comprising at least one receiver or transceiver.

31. The drug delivery device of claim 30, wherein the receiver is configured to receive at least one signal including one or more of an electromagnetic signal, radio-frequency signal, optical signal, acoustic signal, ultrasonic signal, electrical signal, or magnetic signal.

32. The drug delivery device of claim 1, further comprising at least one imaging apparatus capable of imaging the levels of the one or more biological signaling molecules within a therapeutically effective region.

33. The drug delivery device of claim 1, wherein the at least one reservoir includes one or more inlet mechanisms for receiving external delivery of the at least one therapeutic composition.

34. The drug delivery device of claim 1, further comprising at least one memory location for recording information.

35. The drug delivery device of claim 34, wherein the at least one memory location is configured to record information regarding at least one sensor.

36. The drug delivery device of claim 34, wherein the at least one memory location is configured to record information regarding at least one of a sensed condition, history, or performance of the device.

37. The drug delivery device of claim 34, wherein the at least one memory location is configured to record information regarding at least one of the date, time, quantity of material delivered, presence of one or more biological signaling molecules, or level of one or more biological signaling molecules.

38. The drug delivery device of claim 1, further comprising a time-release regulator for the release over time of the at least one therapeutic composition.

39. The drug delivery device of claim 1, further comprising a receiver configured to obtain release instructions or authorization to release the at least one therapeutic composition.

40. The drug delivery device of claim 1, wherein the device includes at least one of a stent, shunt, iontophoretic, patch, or depot.

41. The drug delivery device of claim 1, wherein the first formulation is included in at least one different reservoir than the second formulation.

42. The drug delivery device of claim 1, wherein the device is configured to release the first formulation and the second formulation at different rates or time points.

43. An article of manufacture, comprising:
an article configured to contact at least one biological tissue of a subject;
and configured to deliver at least one therapeutic composition including at least one virus entry inhibitor and at least one viral-replication modulator;
wherein the maximum concentration in a biological fluid of the at least one virus entry inhibitor occurs at a time point approximately prior to a time point at which the at least one viral-replication modulator reaches a maximum concentration.

44. The article of manufacture of claim 43, wherein the article includes at least one of a glove; bottle nipple; bib; pacifier; food dish or food dish cover; bandage; surgical drape; adult, child, or baby diaper; toy or toy cover; chair or chair cover; seat or seat cover; medical gown; blanket; door handle or door handle cover; light switch or light switch cover; keyboard or keyboard cover; hand sanitizing material; clothing; utensil or utensil cover; condom; vaginal sponge; diaphragm; cervical cap; vaginal ring; suppository; douche; enema; body cavity insert; contact lens; dental implant; dental accessory; or paper product.

45. The article of manufacture of claim 44, wherein the body cavity insert includes an ear plug or a nose plug.

46. The article of manufacture of claim 44, wherein the at least one paper product includes at least one of a cup; bowl; plate; paper towel; adult, child, or baby diaper; tampon; tissue paper; absorbent pad; bandage; surgical drape; medical gown; mask; blanket; bib; or other paper product.

47. The article of manufacture of claim 43, wherein the article is at least one of reusable or disposable.

48. The article of manufacture of claim 43, wherein the at least one virus entry inhibitor is present at a higher concentration than the at least one viral-replication modulator.
